(19) United States
(12) Patent Application Publication
(10) Pub. No.: US 2016/0168237 A1
(43) Pub. Date: Jun. 16, 2016

(54) METHOD FOR TREATING A COMPLEMENT MEDIATED DISORDER CAUSED BY AN INFECTIOUS AGENT IN A PATIENT

(71) Applicant: Alexion Pharmaceuticals, Inc., Cheshire, CT (US)

(72) Inventors: Darrell Fontenot, New Haven, CT (US); Bridget Puffer, Cheshire, CT (US); Bruce Andrien, Guilford, CT (US); Mittie Doyle, Dedham, MA (US); Paul Tamburini, Kensington, CT (US); Camille Bedrosian, Cheshire, CT (US)

(21) Appl. No.: 14/966,921

(22) Filed: Dec. 11, 2015

Related U.S. Application Data
(60) Provisional application No. 62/091,073, filed on Dec. 12, 2014.
(60) Provisional application No. 62/127,003, filed on Mar. 2, 2015.
(60) Provisional application No. 62/217,425, filed on Sep. 11, 2015.

(51) Int. Cl. C07K 16/18; A61K 39/395

(52) U.S. Cl. CPC C07K 16/18; A61K 39/3955; C07K 2317/565

(57) ABSTRACT

The present disclosure relates to, inter alia, a method of treating a complement mediated disorder caused by an infectious agents in a patient, comprising administering an effective amount of a C5 inhibitor, such as eculizumab or an eculizumab variant, to the patient.

### INCORPORATION OF SEQUENCE LISTING

The instant application contains a Sequence Listing, which has been submitted electronically in ASCII format and is hereby incorporated by reference in its entirety. Said ASCII copy, created on Dec. 10, 2015, is named 0247US_SL.txt and is 53,858 bytes in size.

### TECHNICAL FIELD

This invention relates to the fields of immunology and infectious disease.

### BACKGROUND

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, in The Merck Manual, 16th Edition.

While a properly functioning complement system provides a robust defense against infecting microbes, inappropriate regulation or activation of the complement pathways has been implicated in the pathogenesis of a variety of disorders, including disorders caused by an infectious agent, including: Shiga toxin-producing E. coli hemolytic uremic syndrome (STEC-HUS), a disease characterized by systemic complement-mediated thrombotic microangiopathy (TMA) and acute vital organ damage; sepsis, a life-threatening medical condition caused by complication of infection, resulting in one or more types of microorganisms entering the human bloodstream and triggering an uncontrolled inflammatory response; and hemorrhagic fever, such as Ebola hemorrhagic fever (EHF).

### SUMMARY

This disclosure provides a method of treating a complement mediated disorder caused by an infectious agent in a patient comprising administering an effective amount of an inhibitor of complement C5 protein to the patient.

In certain aspects, a method is provided of treating a complement mediated disorder caused by a virus that can cause hemorrhagic fever in a patient (i.e., a VHF in a patient; or a patient with a hemorrhagic fever virus infection), comprising administering an effective amount of an inhibitor of a complement C5 protein (“a C5 inhibitor”) to the patient.

In certain aspects, a method is provided of treating sepsis in a patient, comprising determining that the C5a level is elevated in the patient, and administering an effective amount of a C5 inhibitor, such as, for example, eculizumab, an antigen-binding fragment thereof, an antigen-binding variant thereof (also referred to herein as an eculizumab variant or a variant eculizumab, or the like), a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, to the patient.

In certain aspects, a method is provided of treating a human patient with Shiga toxin-producing E. coli hemolytic uremic syndrome (STEC-HUS), the method comprising administering to the patient an effective amount of an anti-C5 antibody, or antigen binding fragment thereof, wherein the method comprises an administration cycle comprising an induction phase followed by a maintenance phase, wherein:
the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or
the anti-C5 antibody, or antigen binding fragment thereof, is administered during the induction phase at a dose of 600 mg weekly for 1 week, starting at day 0, and is administered during the maintenance phase at a dose of 600 mg every week; or
the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1 shows a design of a study protocol.

FIG. 2 shows a study design and schedule of assessments/observations.

FIG. 3 shows sub-study at select site(s) design and schedule of assessments/observations.

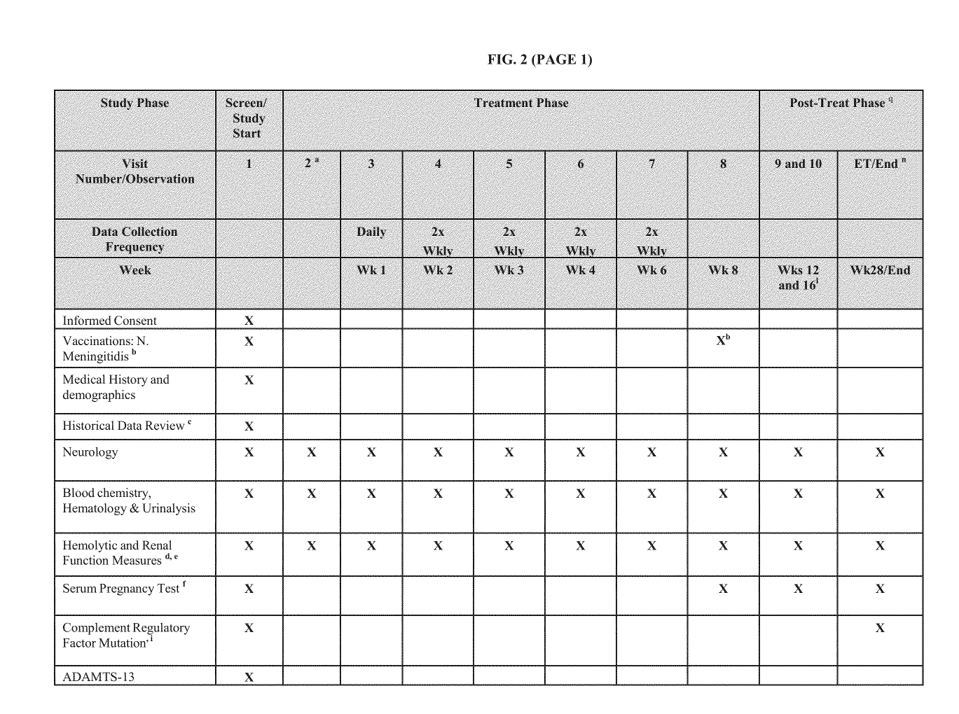

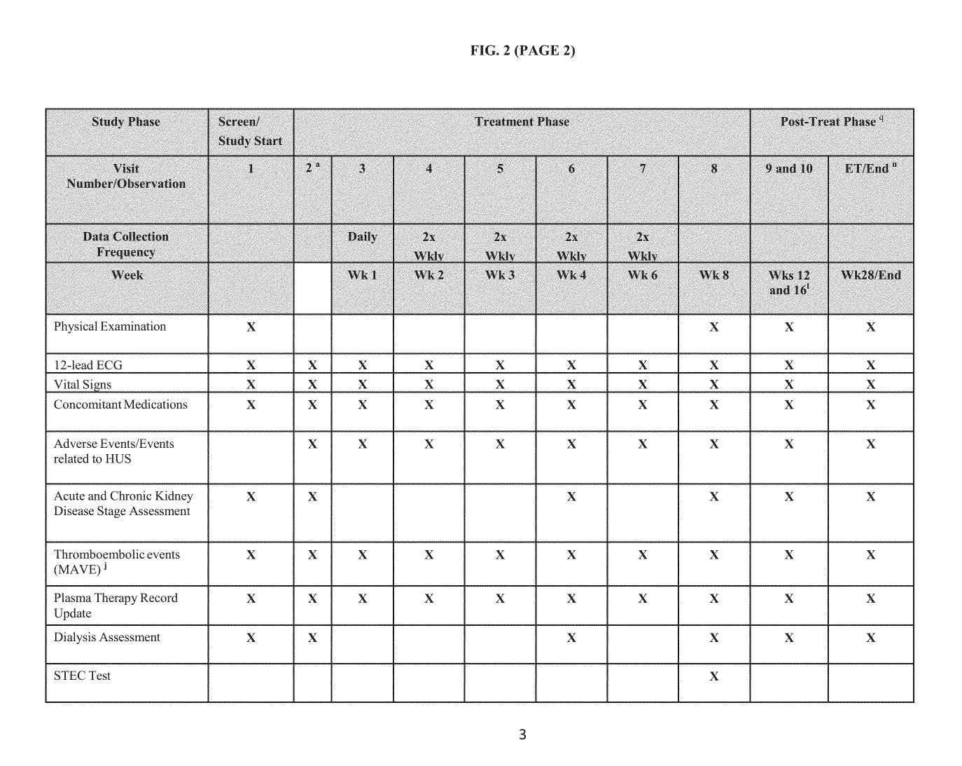

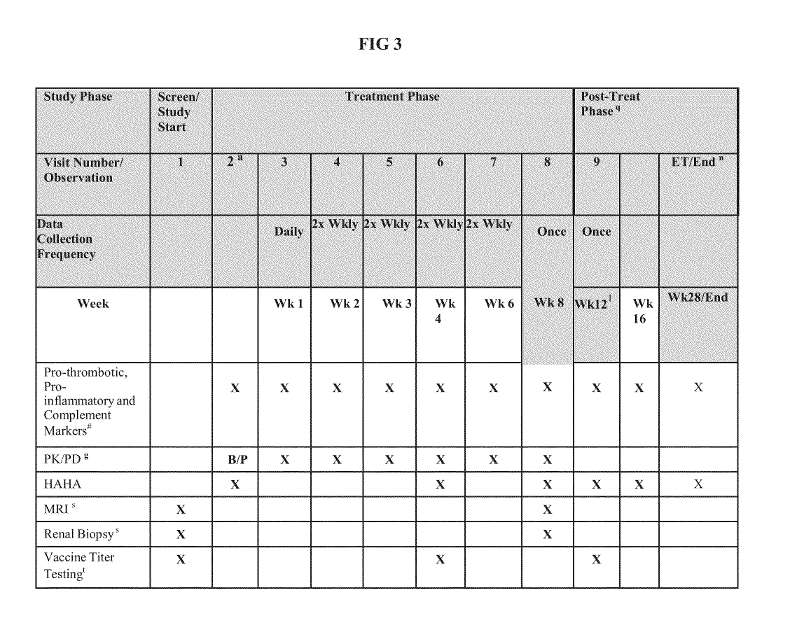

The following refer to both FIG. 2 and FIG. 3; the tags (a, b, #, etc.) are in the figures.

a Visit 1 and 2 may be combined. b If all eligibility criteria are met all patients are vaccinated against meningococcal infection with a quadrivalent meningococcal conjugate vaccine (preferably Menveo®), unless previously vaccinated against meningococcal infection, AND all patients who continue treatment with eculizumab beyond 8 weeks receive a booster vaccination with a quadrivalent meningococcal conjugate vaccine (preferably Menveo®) at week 8. Moreover all patients will receive prophylactic antibiotic (azithromycin or age-appropriate antibiotics) until 14 days after initial vaccination. c Historical data review are completed for each patient and recorded on the CRF. d Hemolytic markers (Serum LDH, plasma haptoglobin, plasma free hemoglobin, reticulocytes, #schistocytes/HPF and presence or absence of schistocytes). e Renal function measures (serum creatinine, urinary protein/creatinine ratio, eGFR). # Prothrombotic, proinflammatory and complement assessments and additional exploratory markers can be assessed. f Pregnancy tests must be performed on all women who have achieved menarche at Visits 1 and ET. Pregnancy tests may also be performed at any visit at the PI's discretion. g Baseline (B) sample for PK and PD testing is to be taken 5-90 minutes before eculizumab infusion. Peak (P) sample for PK and PD testing are to be taken 60 minutes after the completion of eculizumab infusion. i Complement Regulatory Factor Mutation analysis can be performed. j Information on thromboembolic events—large and small vessel thrombosis as well as microthrombosis (major adverse vascular events; MAVE), including date location and method of diagnosis. k Unscheduled visit for plasma therapy intervention to include collection of platelet counts immediately prior to PE/PPH administration and 24 hours after PE/PPH administration. Also collect hemoglobin and hematocrit, WBC counts, BUN, LDH, haptoglobin, serum creatinine, urinalysis and urine protein/creatinine ratio immediately prior to PE/PPH administration and 24 hours after PE/PPH administration. l Patients who continue eculizumab will have additional visits at week 10 and 14. m To be completed on PE/PPH visits only. n Assessments requested at Week 28 need to occur at early termination or end of study. o PK may be drawn if needed to enable PK analysis in the event of unexpected toxicity and/or loss of efficacy. q Patients who discontinue eculizumab treatment then are followed for the full 28 weeks to assess STEC-HUS outcomes. r Test to be used to assess continuing treatment strategies. s Test to be used to assess continuing treatment strategies. t Approximately 50 patients, from all age groups will be selected randomly and in a prospective manner, can be studied to understand the effect of giving a booster vaccine on SBA titers: Blood draws will be done prior to vaccination, at week 4 post vaccination, and again at 12 weeks post vaccination. Samples are measurement for SBA using the baby rabbit assay.

### DETAILED DESCRIPTION

As used herein, the word “a” or “plurality” before a noun represents one or more of the particular noun. For example, the phrase “a mammalian cell” represents “one or more mammalian cells.”

The term “recombinant protein” is known in the art. Briefly, the term “recombinant protein” can refer to a protein that can be manufactured using a cell culture system. The cells in the cell culture system can be derived from, for example, a mammalian cell, including a human cell, an insect cell, a yeast cell, or a bacterial cell. In general, the cells in the cell culture contain an introduced nucleic acid encoding the recombinant protein of interest (which nucleic acid can be borne on a vector, such as a plasmid vector). The nucleic acid encoding the recombinant protein can also contain a heterologous promoter operably linked to a nucleic acid encoding the protein.

The term “mammalian cell” is known in the art and can refer to any cell from or derived from any mammal including, for example, a human, a hamster, a mouse, a green monkey, a rat, a pig, a cow, a hamster, or a rabbit. In some embodiments, the mammalian cell can be an immortalized cell, a differentiated cell, or an undifferentiated cell.

The term “immunoglobulin” is known in the art. Briefly, the term “immunoglobulin” can refer to a polypeptide containing an amino acid sequence of at least 15 amino acids (e.g., at least 20, 30, 40, 50, 60, 70, 80, 90, or 100 amino acids, or more than 100 amino acids) of an immunoglobulin protein (e.g., a variable domain sequence, a framework sequence, or a constant domain sequence). The immunoglobulin can, for example, include at least 15 amino acids of a light chain immunoglobulin, e.g., at least 15 amino acids of a heavy chain immunoglobulin, such as a CDRH3. The immunoglobulin may be an isolated antibody (e.g., an IgG, IgE, IgD, IgA, or IgM). The immunoglobulin may be a subclass of IgG (e.g., IgG1, IgG2, IgG3, or IgG4). The immunoglobulin can be an antibody fragment, e.g., a Fab fragment, a F(ab′)₂ fragment, or a scFv. The immunoglobulin can also be an engineered protein containing at least one immunoglobulin domain (e.g., a fusion protein). The engineered protein or immunoglobulin-like protein can also be a bi-specific antibody or a tri-specific antibody, or a dimer, trimer, or multimer antibody, or a diabody, a DVD-Ig, a CODV-Ig, an Affibody®, or a Nanobody®. Non-limiting examples of immunoglobulins are described herein and additional examples of immunoglobulins are known in the art.

The term “engineered protein” is known in the art. Briefly, the term “engineered protein” can refer to a polypeptide that is not naturally encoded by an endogenous nucleic acid present within an organism (e.g., a mammal). Examples of engineered proteins include modified enzymes with one or more amino acid substitutions, deletions, insertions, or additions that result in an increase in stability and/or catalytic activity of the engineered enzyme, fusion proteins, humanized antibodies, chimeric antibodies, divalent antibodies, trivalent antibodies, four binding domain antibodies, a diabody, and antigen-binding proteins that contain at least one recombinant scaffolding sequence.

The terms “polypeptide,” “peptide,” and “protein” are used interchangeably and are known in the art and can mean any peptide-bond linked chain of amino acids, regardless of length or post-translational modification.

The term “antibody” is known in the art. The term “antibody” is sometimes used interchangeably with the term “immunoglobulin.” Briefly, it can refer to a whole antibody comprising two light chain polypeptides and two heavy chain polypeptides. Whole antibodies include different antibody isotypes including IgM, IgG, IgA, IgD, and IgE antibodies. The term “antibody” includes, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody. The antibody can also be an engineered protein or antibody-like protein containing at least one immunoglobulin domain (e.g., a fusion protein). The engineered protein or antibody-like protein can also be a bi-specific antibody or a tri-specific antibody, or a dimer, trimer, or multimer antibody, or a diabody, a DVD-Ig, a CODV-Ig, an Affibody®, or a Nanobody®.

The term “antibody fragment,” “antigen-binding fragment,” or similar terms are known in the art and can, for example, refer to a fragment of an antibody that retains the ability to bind to a target antigen (e.g., human C5) and inhibit the activity of the target antigen. Such fragments include, e.g., a single chain antibody, a single chain Fv fragment (scFv), an Fd fragment, a Fab fragment, a Fab′ fragment, or an F(ab′)2 fragment. A scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283. An antigen-binding fragment can also include the variable region of a heavy chain polypeptide and the variable region of a light chain polypeptide. An antigen-binding fragment can thus comprise the CDRs of the light chain and heavy chain polypeptide of an antibody.

The term “antibody fragment” also can include, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079. The term “antibody fragment” also includes single domain antibodies comprising two V_{H} domains with modifications such that single domain antibodies are formed.

The term “kₐ” is well known in the art and can refer to the rate constant for association of an antibody to an antigen. The term “k_{d}” is also well known in the art and can refer to the rate constant for dissociation of an antibody from the antibody/antigen complex. And the term “K_{D}” is known in the art and can refer to the equilibrium dissociation constant of an antibody-antigen interaction. The equilibrium dissociation constant is deduced from the ratio of the kinetic rate constants, K_{D}=kₐ/k_{d}. Such determinations are typically measured at, for example, 25° C. or 37° C. For example, the kinetics of antibody binding to human C5 can be determined at pH 8.0, 7.4, 7.0, 6.5 and 6.0 via surface plasmon resonance (“SPR”) on a BIAcore 3000 instrument using an anti-Fc capture method to immobilize the antibody.

As used herein, the terms “induction” and “induction phase” are used interchangeably and refer to the first phase of treatment in a clinical trial.

As used herein, the terms “maintenance” and “maintenance phase” are used interchangeably and refer to the second phase of treatment in a clinical trial. In certain embodiments, treatment is continued as long as clinical benefit is observed or until unmanageable toxicity or disease progression occurs.

As used herein, the term “subject” and “patient” are used interchangeably. A patient or a subject can be a human patient or a human subject.

As used herein, “effective treatment” refers to treatment producing a beneficial effect, e.g., amelioration of at least one symptom of a disease or disorder in a patient. A beneficial effect can take the form of an improvement over baseline, i.e., an improvement over a measurement or observation made prior to initiation of therapy according to the method.

In certain embodiments, for treating a patient with STEC-HUS, effective treatment may refer to alleviation of at least one symptom of STEC-HUS (e.g., TMA, renal failure, neurological symptoms, elevated LDH level, elevated hemoglobin level, or elevated platelet count).

In certain embodiments, effective treatment may refer to that improves the patient's chance of survival. In certain embodiments, a disclosed method improves the life expectancy of a patient by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

The term “effective amount” or “a therapeutically effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease in a patient, or any other desired alteration of a biological system. An effective amount can be administered in one or more administrations. In certain other embodiments, an “effective amount” or “a therapeutically effective amount” is the amount of a C5 inhibitor, such as an anti-C5 antibody, or antigen binding fragment thereof, that improves the life expectancy of a patient by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

In certain embodiments, for treating a patient with STEC-HUS, an “effective amount” or “a therapeutically effective amount” is the amount of anti-C5 antibody, or antigen binding fragment thereof, clinically proven to alleviate at least one symptom of STEC-HUS (e.g., TMA, renal failure, neurological symptoms, elevated LDH level, elevated hemoglobin level, or elevated platelet count).

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The Complement System

As is well known, the complement system acts in conjunction with other immunological systems of the body to defend against intrusion of cellular and viral pathogens. There are at least 25 complement proteins. Complement components achieve their immune defensive functions by interacting in a series of intricate but precise enzymatic cleavage and membrane binding events. The resulting complement cascade leads to the production of products with opsonic, immunoregulatory, and lytic functions.

The complement cascade can progress via the classical pathway (“CP”), the lectin pathway, or the alternative pathway (“AP”). The lectin pathway is typically initiated with binding of mannose-binding lectin (“MBL”) to high mannose substrates. The AP can be antibody independent, and can be initiated by certain molecules on pathogen surfaces. The CP is typically initiated by antibody recognition of, and binding to, an antigenic site on a target cell. These pathways converge at the C3 convertase—the point where complement component C3 is cleaved by an active protease to yield C3a and C3b.

The AP C3 convertase is initiated by the spontaneous hydrolysis of complement component C3, which is abundant in the plasma in the blood. This process, also known as “tickover,” occurs through the spontaneous cleavage of a thioester bond in C3 to form C3i or C3(H₂O). Tickover is facilitated by the presence of surfaces that support the binding of activated C3 and/or have neutral or positive charge characteristics (e.g., bacterial cell surfaces). This formation of C3(H₂O) allows for the binding of plasma protein Factor B, which in turn allows Factor D to cleave Factor B into Ba and Bb. The Bb fragment remains bound to C3 to form a complex containing C3(H₂O)Bb—the “fluid-phase” or “initiation” C3 convertase. Although only produced in small amounts, the fluid-phase C3 convertase can cleave multiple C3 proteins into C3a and C3b and results in the generation of C3b and its subsequent covalent binding to a surface (e.g., a bacterial surface). Factor B bound to the surface-bound C3b is cleaved by Factor D to thus form the surface-bound AP C3 convertase complex containing C3b,Bb. See, e.g., Müller-Eberhard (1988) Ann Rev Biochem 57:321-347.

The AP C5 convertase—(C3b)₂,Bb—is formed upon addition of a second C3b monomer to the AP C3 convertase. See, e.g., Medicus et al. (1976) J Exp Med 144:1076-1093 and Fearon et al. (1975) J Exp Med 142:856-863. The role of the second C3b molecule is to bind C5 and present it for cleavage by Bb. See, e.g., Isenman et al. (1980) J Immunol 124:326-331. The AP C3 and C5 convertases are stabilized by the addition of the trimeric protein properdin as described in, e.g., Medicus et al. (1976), supra. However, properdin binding is not required to form a functioning alternative pathway C3 or C5 convertase. See, e.g., Schreiber et al. (1978) Proc Natl Acad Sci USA 75: 3948-3952, and Sissons et al. (1980) Proc Natl Acad Sci USA 77: 559-562.

The CP C3 convertase is formed upon interaction of complement component C1, which is a complex of C1q, C1r, and C1s, with an antibody that is bound to a target antigen (e.g., a microbial antigen). The binding of the C1q portion of C1 to the antibody-antigen complex causes a conformational change in C1 that activates C1r. Active C1r then cleaves the C1-associated C1s to thereby generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial cell surface). C1s also cleaves complement component C2 into C2b and C2a. The complex formed by C4b and C2a is the CP C3 convertase, which is capable of processing C3 into C3a and C3b. The CP C5 convertase—C4b,C2a,C3b—is formed upon addition of a C3b monomer to the CP C3 convertase. See, e.g., Müller-Eberhard (1988), supra and Cooper et al. (1970) J Exp Med 132:775-793.

In addition to its role in C3 and C5 convertases, C3b also functions as an opsonin through its interaction with complement receptors present on the surfaces of antigen-presenting cells such as macrophages and dendritic cells. The opsonic function of C3b is generally considered to be one of the most important anti-infective functions of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone.

The AP and CP C5 convertases cleave C5, which is a 190 kDa beta globulin found in normal human serum at approximately 75 μg/ml (0.4 μM). C5 is glycosylated, with about 1.5-3 percent of its mass attributed to carbohydrate. Mature C5 is a heterodimer of a 999 amino acid 115 kDa alpha chain that is disulfide linked to a 655 amino acid 75 kDa beta chain. C5 is synthesized as a single chain precursor protein product of a single copy gene (Haviland et al. (1991) J Immunol. 146:362-368). The cDNA sequence of the transcript of this human gene predicts a secreted pro-C5 precursor of 1658 amino acids along with an 18 amino acid leader sequence. See, e.g., U.S. Pat. No. 6,355,245.

The pro-C5 precursor is cleaved after amino acids 655 and 659, to yield the beta chain as an amino terminal fragment (amino acid residues +1 to 655 of the above sequence) and the alpha chain as a carboxyl terminal fragment (amino acid residues 660 to 1658 of the above sequence), with four amino acids (amino acid residues 656-659 of the above sequence) deleted between the two.

C5a is cleaved from the alpha chain of C5 by either alternative or classical C5 convertase as an amino terminal fragment comprising the first 74 amino acids of the alpha chain (i.e., amino acid residues 660-733 of the above sequence). Approximately 20 percent of the 11 kDa mass of C5a is attributed to carbohydrate. The cleavage site for convertase action is at, or immediately adjacent to, amino acid residue 733. A compound that would bind at, or adjacent to, this cleavage site would have the potential to block access of the C5 convertase enzymes to the cleavage site and thereby act as a complement inhibitor. A compound that binds to C5 at a site distal to the cleavage site could also have the potential to block C5 cleavage, for example, by way of steric hindrance-mediated inhibition of the interaction between C5 and the C5 convertase. A compound, in a mechanism of action consistent with that of the tick saliva complement inhibitor, Ornithodoros moubata C inhibitor (‘OmCI”) (which can be a C5 inhibitor that can be used in the methods of this invention), may also prevent C5 cleavage by reducing flexibility of the C345C domain of the alpha chain of C5, which reduces access of the C5 convertase to the cleavage site of C5. See, e.g., Fredslund et al. (2008) Nat Immunol 9(7):753-760.

C5 can also be activated by means other than C5 convertase activity. Limited trypsin digestion (see, e.g., Minta and Man (1997) J Immunol 119:1597-1602 and Wetsel and Kolb (1982) J Immunol 128:2209-2216) and acid treatment (Yamamoto and Gewurz (1978) J Immunol 120:2008 and Damerau et al. (1989) Molec Immunol 26:1133-1142) can also cleave C5 and produce active C5b.

Cleavage of C5 releases C5a, a potent anaphylatoxin and chemotactic factor, and leads to the formation of the lytic terminal complement complex, C5b-9. C5a and C5b-9 also have pleiotropic cell activating properties, by amplifying the release of downstream inflammatory factors, such as hydrolytic enzymes, reactive oxygen species, arachidonic acid metabolites and various cytokines.

The first step in the formation of the terminal complement complex involves the combination of C5b with C6, C7, and C8 to form the C5b-8 complex at the surface of the target cell. Upon the binding of the C5b-8 complex with several C9 molecules, the membrane attack complex (“MAC”, C5b-9, terminal complement complex—“TCC”) is formed. When sufficient numbers of MACs insert into target cell membranes the openings they create (MAC pores) mediate rapid osmotic lysis of the target cells, such as red blood cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis.

C3a and C5a are anaphylatoxins. These activated complement components can trigger mast cell degranulation, which releases histamine from basophils and mast cells, and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

C5a receptors are found on the surfaces of bronchial and alveolar epithelial cells and bronchial smooth muscle cells. C5a receptors have also been found on eosinophils, mast cells, monocytes, neutrophils, and activated lymphocytes.

While a properly functioning complement system provides a robust defense against infecting microbes, inappropriate regulation or activation of complement has been implicated in the pathogenesis of a variety of disorders, including, e.g., rheumatoid arthritis; lupus nephritis; asthma; ischemia-reperfusion injury; atypical hemolytic uremic syndrome (“aHUS”); dense deposit disease; paroxysmal nocturnal hemoglobinuria (PNH); macular degeneration (e.g., age-related macular degeneration; hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis. See, e.g., Holers et al. (2008) Immunological Reviews 223:300-316.

Treating Patients with Complement Mediated Disorders Caused by an Infectious Agent

Inappropriate regulation or activation of the complement pathways may also be implicated in the pathogenesis of infectious diseases in patients, including Shiga toxin-producing E. coli hemolytic uremic syndrome (STEC-HUS), a disease characterized by systemic complement-mediated thrombotic microangiopathy (TMA) and acute vital organ damage; sepsis, a life-threatening medical condition caused by complication of infection, resulting in one or more types of microorganisms entering the human bloodstream and triggering an uncontrolled inflammatory response; and hemorrhagic fever, such as Ebola hemorrhagic fever (EHF). Thus, these are examples of complement mediated disorders caused by infectious agents.

Hemorrhagic fever, such as Ebola hemorrhagic fever (“EHF”), is an infectious disease in a patient caused by an enveloped RNA virus, thus the name viral hemorrhagic fever (VHF). Four families of RNA viruses are viruses that can cause VHF in humans; these are the filoviruses (Ebola being an example) of the taxonomic family Filoviridae, the flaviviruses of the taxonomic family Flavivirudae, the arenaviruses of the taxonomic family Arenavirirudae, and the bunyaviruses of the taxonomic family Bunyavirudae. Not all viruses in these families cause VHF; but many can. Patients often present with severe internal bleeding, including hemolysis. Patients suffering from viral hemorrhagic fever have also presented with thrombolitic microangiopathy and acute renal failure. See, e.g., Ardalan et al., Nephrol Dial Transplant (2006) 21: 2304-2307. The pathogenic mechanisms of VHF include over-production of certain cytokines, disseminated intravascular coagulation, and complement activation. See, e.g., Paessler and Walker, Ann. Rev. Pathol. Mech. Dis. 2013, 8: 411-440. Antibody-dependent and complement-component-C1q dependent enhancement of Ebola virus infection has been reported. Takada et al., Journal of Virology, July 2003, 77(3), p. 7539-7544. DOI:10.1128/JVI.77.13.7539-7544.2003.

Sepsis is a life-treating medical condition. It is caused by complication of infection, resulting in one or more types of microorganisms entering the human bloodstream and triggering an uncontrolled inflammatory response. Sepsis occurs when chemicals released into the bloodstream to fight the infection trigger inflammatory responses throughout the body, including, for example, over-production of proinflammatory cytokines, such as IL-6, IL-17, TNFα, and integrin α₃β₁. See, e.g., Weaver et al., the FASEB J, 2004, 18, pp. 1185-1191; Xu et al., 2010, Eur. J. Immunol., 40: 1079-1088; Rierdemann et al., 2003, J Immunol. 170: 503-507; Lerman et al., Blood, 2014, 124(24): 3515-3523. Sepsis can also result in increased serum LDH level in a patient, which can be accompanied by increased lactic acid level, SGOT level, creatine kinase level, or creatine level, or by increased platelet count or increased plasma bicarbonate level. Zein et al., Chest, 2004; 126(4_meetingAbstracts):873S. doi:10.1378/chest.126.4_MeetingAbstracts.873S. This inflammation can trigger a cascade of changes that can damage multiple organ systems, causing them to fail. See, e.g., Xu et al., 2010, Eur. J. Immunol., 40: 1079-1088. Also, the complement system is over-activated in sepsis, resulting in excessive production of the complement protein C5a. See, e.g., Xu et al., 2010, Eur. J. Immunol., 40: 1079-1088; Flierl et al., J of Investigative Surgery, 19: 255-265, 2006; Gao et al., the FASEB J, 2005, 19(8): 1003-5, 10.1096/fj.04-3424fje; Guo et al., SHOCK, 2004, 21(1): 1-7, 2004; Rierdemann et al., 2003, J Immunol. 170: 503-507; Lerman et al., Blood, 2014, 124(24);Rierdamann et al., J of Leukocyte Biology 74: 966-970, 2003; Sprong et al., Blood, 2003, 102(10): 3702-3710. Septic shock, brought about by a drop in blood pressure and a weakened heart, is the most severe complication of sepsis and can be deadly. “Sepsis Questions and Answers”, cdc.gov. Centers for Disease Control and Prevention (CDC), May 22, 2014 (http://www.cdc.gov/sepsis/basic/qa.html).

Many types of infections can lead to sepsis in a patient, including infections of the skin, lungs, urinary tract, abdomen (such as appendicitis), or other part of the body. Pneumonia, central line-associated bloodstream infections, catheter-associated urinary tract infections, and surgical site infections can also sometimes lead to sepsis. MRSA infections of the skin and soft tissue can also lead to sepsis. “Sepsis Questions and Answers”. cdc.gov. Centers for Disease Control and Prevention (CDC). May 22, 2014 (http://www.cdc.gov/sepsis/basic/qa.html). Common symptoms of sepsis include, for example, fever, chills, rapid breathing and heart rate, rash, confusion, and disorientation. Id. Sepsis can be diagnosed by methods known in the art, such as by the use of microbial cultures. Bacterial, fungal, or viral infection can lead to sepsis.

Shiga-like toxin-producing Escherichia coli (STEC) is a pathogen that has recently infected human patients in Germany and other countries to near epidemic levels. Many of those who are infected have developed STEC-HUS and are critically ill due to uncontrolled complement activation, leading to systemic thrombotic microangiopathy (TMA), the underlying pathological mechanism of HUS and for which there is no approved therapy. STEC-HUS is the most common cause of renal failure in childhood, accounting for >90% of HUS cases, and is difficult to treat with current therapeutic modalities, and often leads to persistent renal damage. Severe central nervous system involvement is another manifestation of STEC-HUS and, though historically rare, has been reported to be a frequent clinical complication in the 2011 STEC-HUS cases in Germany, and often leads to death or permanent neurological damage. Severe and uncontrolled complement activation caused by STEC-HUS is difficult to manage with current therapeutic modalities. There is limited medical evidence that plasma infusion or exchange therapies improve outcomes of STEC-induced HUS. Multiple reports from German physicians indicate that plasma support is ineffective. Uncontrolled complement activation can extend from at least weeks to months following the initial presentation of STEC-HUS.

In certain aspects, a method is provided of treating a complement mediated disorder caused by an infectious agent in a patient (such as a human patient) comprising administering an effective amount of a polypeptide inhibitor of complement C5 protein (such as human complement C5 protein) to the patient.

In certain embodiments, the infectious agent can be viruses, bacteria, protozoa, fungi, prions and worms.

In certain embodiments, the complement mediated disorder is caused by a virus that can cause hemorrhagic fever in a patient. In certain embodiments, the complement mediated disorder is sepsis. In certain embodiments, the complement mediated disorder is STEC-HUS.

In certain embodiments, the complement mediated disorder is any complement mediated disorder caused by an infectious agent. Infectious agent includes, without limitation, bacteria, virus, protozoa, fungi, prions, worms, etc.

Methods of Treating VHF in a Patient

In certain aspects, the infectious agent is a virus that can cause hemorrhagic fever in a patient.

In certain embodiments, a method is provided of treating a complement mediated disorder caused by a virus that can cause hemorrhagic fever in a patient (i.e., a VHF in a patient; or a patient with a hemorrhagic fever virus infection), comprising administering an effective amount of an inhibitor of a complement C5 protein (a “C5 inhibitor”) to the patient.

VHF may be diagnosed by any means, including methods known in the art, or may be suspected.

In certain other embodiments, a method is provided of reducing hemolysis in a patient with a complement mediated disorder caused by a virus that can cause hemorrhagic fever (i.e., a VHF in a patient; or a patient with a hemorrhagic fever virus infection), comprising administering an effective amount of an inhibitor of a complement C5 protein to the patient. In certain embodiments, reduction of hemolysis is determined after the administration of the C5 inhibitor.

In yet certain other embodiments, a method is provided for treating a patient with a complement mediated disorder caused by a virus that can cause hemorrhagic fever (i.e., a VHF in a patient; or a patient with a hemorrhagic fever virus infection), comprising first determining that the complement level is elevated in the patient and then administering an effective amount of an inhibitor of a complement C5 protein to the patient. In some embodiments, once the complement level is reduced to, for example, a normal level, there is no need for further administration of an inhibitor of a complement C5 protein to the patient and thus the patient is not subjected to further administration of an inhibitor of a complement C5 protein. The complement level can be considered elevated, for example, if it is a level that can be harmful to the patient, or, for another example, a level that is elevated compared to the normal level of complement in that patient, or normal level for a patient based on size, age, etc. Normal level of complement in a patient can mean a level that is not harmful to the patient, or, for another example, a level that is elevated compared to the normal level of complement in that patient, or normal level for a patient based on size, age, etc. In some embodiments, the method further comprises the patient experiencing a reduction in hemolysis, after receiving treatment with the C5 inhibitor. Reduction of hemolysis can be monitored by any methods known in the art.

In certain embodiments, whether the complement level is elevated in the patient is first determined prior to administering a C5 inhibitor to that patient. In certain further embodiments, once the complement level is reduced to, for example, a normal level, there is no need for further administration of an inhibitor of a complement C5 protein to the patient and thus the patient is not subjected to further administration of an inhibitor of a complement C5 protein. The level of complement in a patient can be determined by any methods known in the art. Too low a level of complement may be associated with enhancement of Ebola virus infection. See Brudner et al., (2013) PLoS ONE 8(4): e60838. doi:10.1371/journal.pone.0060838.

In certain embodiments, the patient is treated as early in his or her infection by a virus that can cause VHF as possible with a C5 inhibitor, including an anti-C5 antibody.

The frequency of administration can also be adjusted according to various parameters. These include, for example, the clinical response, the plasma half-life of the C5 inhibitor, and the levels of the C5 inhibitor (such as an antibody) in a body fluid, such as, blood, plasma, serum, or synovial fluid. To guide adjustment of the frequency of administration, levels of the C5 inhibitor in the body fluid can be monitored during the course of treatment.

In certain embodiments, the frequency of administration may be adjusted according to an assay measuring cell-lysing ability of complement present in one or more of the patient's body fluids. The cell-lysing ability can be measured as percent hemolysis in hemolytic assays known in the art. An about 10% or about 25% or about 50% reduction in the cell-lysing ability of complement present in a body fluid after treatment with the antibody capable of inhibiting complement used in the practice of the application means that the percent hemolysis after treatment is about 90, about 75, or about 50 percent, respectively, of the percent hemolysis before treatment.

For the treatment of VHF by systemic administration of a C5 inhibitor, such as, for example, an antibody, administration of a large initial dose can be performed, i.e., a single initial dose sufficient to yield a substantial reduction, and more preferably an at least about 50% reduction, in the hemolytic activity of the patient's serum. Such a large initial dose can be followed by regularly repeated administration of tapered doses as needed to maintain substantial reductions of serum hemolytic titer. In other embodiments, the initial dose is given by both local and systemic routes, followed by repeated systemic administration of tapered doses as described above.

In certain embodiments, a VHF patient receives 900 milligrams (mg) of eculizumab each week for the first 3 weeks, followed by a 1200 mg dose on weeks 4, 6, and 8. After an initial 8-week eculizumab treatment period, the patient can optionally receive further treatment with eculizumab 1200 mg every other week, up to an additional 8 weeks.

The patient to be treated is a patient infected with a virus that can cause VHF. In certain embodiments, the patient is suffering from internal bleeding, which can be severe. In certain embodiments, the patient is experiencing thrombolitic microangiopathy or acute renal failure. In certain embodiments, the patient is experiencing over-production of certain cytokines, disseminated intravascular coagulation, or complement activation. In certain embodiments, the patient is experiencing antibody-dependent and complement-component-C1q dependent enhancement of Ebola virus infection. In certain embodiments, the patient is suffering from one or more symptoms of VHF. The diagnosis of the disease, as well as methods for diagnosing the symptoms above, are known in the art.

A virus that can cause hemorrhagic fever can be a filovirus, a flavivirus, an arenavirus, or a bunyavirus. A virus that can cause hemorrhagic fever can be any of the four families of RNA viruses that can cause VHF in humans; these are the filoviruses (Ebola being an example) of the taxonomic family Filoviridae, the flaviviruses of the taxonomic family Flavivirudae, the arenaviruses of the taxonomic family Arenavirirudae, and the bunyaviruses of the taxonomic family Bunyavirudae. In certain embodiments, the virus is a filovirus. In further embodiments, the filovirus is an Ebola virus. Many genus and species of these viruses can cause viral hemorrhagic fever. The Ebola virus and the Marburg virus are examples of filoviruses. The genus Ebola virus, causing Ebola hemorrhagic fever, has, at this point, five different species: Zaire ebolavirus, Sudan ebolavirus, Reston ebolavirus, Cote d'Ivoire ebolavirus and the Bundibugyo ebolavirus. The Yellow fever virus and the Dengue viruses are examples of flaviviruses. The Junin virus and the Machupo virus are examples of Arenaviruses. The Crimean-Congo hemorrhagic fever virus and the Rift valley fever virus are examples of Bunyaviruses.

Outbreaks of Ebola hemorrhagic fever have been problematic. Fatal human patients of Ebola hemorrhagic fever do not mount an effective immune response and do not develop IgG antibodies to the virus; whereas survivors of Ebola hemorrhagic fever develop IgG antibodies, mainly against viral nucleoprotein, early in the course of infection. See, e.g., Paessler and Walker, Ann. Rev. Pathol. Mech. Dis. 2013, 8: 411-440. As stated above, VHF patients often present with internal bleeding, which can be severe, including hemolysis, thrombolitic microangiopathy, and acute renal failure. See, e.g., Ardalan et al., Nephrol Dial Transplant (2006) 21: 2304-2307. The pathogenic mechanisms of VHF include over-production of certain cytokines, disseminated intravascular coagulation, and complement activation. See, e.g., Paessler and Walker, Ann. Rev. Pathol. Mech. Dis. 2013, 8: 411-440. Antibody-dependent and complement-component-C1q dependent enhancement of Ebola virus infection has been reported. Takada et al., Journal of Virology, July 2003, 77(3), p. 7539-7544. DOI:10.1128/JVI.77.13.7539-7544.2003.

In certain embodiments, a therapeutically effective amount of a C5 inhibitor (such as eculizumab) can include an amount (or various amounts in the case of multiple administration) that improves the patient's chance of survival. In certain embodiments, a disclosed method improves the life expectancy of a patient by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

In certain embodiments, a therapeutically effective amount of a C5 inhibitor (such as eculizumab) can include an amount (or various amounts in the case of multiple administration) that decreases hemolysis, decreases disseminated intravascular coagulation, increases platelet levels, reduces complement levels, decreases levels of the cytokines that are over-produced, inhibits thrombolitic microangiopathy, maintains or improves renal functions, or reduces other symptoms of the disease (such as fever), or any combination thereof. These parameters can be ascertained or measured by any methods known in the art.

For example, methods for determining whether a particular C5 inhibitor, such as an anti-C5 antibody, inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2nd Edition,” 135-240, Springfield, Ill., C C Thomas (1961), pages 135-139, or a conventional variation of that assay such as the chicken erythrocyte hemolysis method as described in, e.g., Hillmen et al. (2004) N Engl J Med 350(6):552. Methods for determining whether a compound inhibits the cleavage of human C5 into forms C5a and C5b are known in the art and described in, e.g., Moongkarndi et al. (1982) Immunobiol 162:397; Moongkarndi et al. (1983) Immunobiol 165:323; Isenman et al. (1980) J Immunol 124(1):326-31; Thomas et al. (1996) Mol Immunol 33(17-18):1389-401; and Evans et al. (1995) Mol Immunol 32(16):1183-95. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest 50(3):606-16 and Wurzner et al. (1991) Complement Inflamm 8:328-340). For C5b, hemolytic assays or assays for soluble C5b-9 known in the art can be used. Other assays known in the art can also be used.

Immunological techniques such as, but not limited to, ELISA can be used to measure the protein concentration of C5 and/or its split products to determine the ability of a C5 inhibitor, such as an anti-C5 antibody, to inhibit conversion of C5 into biologically active products. For example, C5a generation can be measured. Also, as another example, C5b-9 neoepitope-specific antibodies can be used to detect the formation of terminal complement.

Hemolytic assays can be used to determine the inhibitory activity of a C5 inhibitor, such as an anti-C5 antibody, on complement activation. In order to determine the effect of a C5 inhibitor, such as an anti-C5 antibody, on classical complement pathway-mediated hemolysis in a serum test solution in vitro, for example, sheep erythrocytes coated with hemolysin or chicken erythrocytes sensitized with anti-chicken erythrocyte antibody can be used as target cells. The percentage of lysis is normalized by considering 100% lysis equal to the lysis occurring in the absence of the inhibitor. Also, the classical complement pathway can be activated by a human IgM antibody, for example, as utilized in the Wieslab® Classical Pathway Complement Kit (Wieslab® COMPL CP310, Euro-Diagnostica, Sweden). Briefly, the test serum is incubated with, for example, a C5 inhibitor such as an anti-C5 antibody in the presence of a human IgM antibody. The amount of C5b-9 that is generated is measured by contacting the mixture with an enzyme conjugated anti-C5b-9 antibody and a fluorogenic substrate and measuring the absorbance at the appropriate wavelength. As a control, the test serum is incubated in the absence of the C5 inhibitor, such as an anti-C5 antibody. In some embodiments, the test serum is a C5-deficient serum reconstituted with a C5 polypeptide.

To determine the effect of a C5 inhibitor, such as an anti-C5 antibody, on alternative pathway-mediated hemolysis, unsensitized rabbit or guinea pig erythrocytes can be used as the target cells. The serum test solution is a C5-deficient serum reconstituted with a C5 inhibitor, such as an anti-C5 polypeptide. The percentage of lysis is normalized by considering 100% lysis equal to the lysis occurring in the absence of the inhibitor. The alternative complement pathway can be activated by lipopolysaccharide molecules, for example, as utilized in the Wieslab® Alternative Pathway Complement Kit (Wieslab® COMPL AP330, Euro-Diagnostica, Sweden). Briefly, the test serum is incubated with a C5 inhibitor, such as an anti-C5 antibody, in the presence of lipopolysaccharide. The amount of C5b-9 that is generated is measured by contacting the mixture with an enzyme conjugated anti-C5b-9 antibody and a fluorogenic substrate and measuring the fluorescence at the appropriate wavelength. As a control, the test serum is incubated in the absence of the C5 inhibitor, such as an anti-C5 antibody.

C5 activity, or inhibition thereof, can be quantified using a CH50eq assay. The CH50eq assay is a method for measuring the total classical complement activity in serum. This test is a lytic assay, which uses antibody-sensitized erythrocytes as the activator of the classical complement pathway and various dilutions of the test serum to determine the amount required to give 50% lysis (CH50). The percent hemolysis can be determined, for example, using a spectrophotometer. The CH50eq assay provides an indirect measure of terminal complement complex (“TCC”) formation, since the TCC themselves are directly responsible for the hemolysis that is measured. The assay is well known and commonly practiced by those skilled in the art.

Briefly and for example, to activate the classical complement pathway, undiluted serum samples (e.g., reconstituted human serum samples) are added to microassay wells containing the antibody-sensitized erythrocytes to thereby generate TCC. Next, the activated sera are diluted in microassay wells, which are coated with a capture reagent (e.g., an antibody that binds to one or more components of the TCC). The TCC present in the activated samples bind to the monoclonal antibodies coating the surface of the microassay wells. The wells are washed and to each well is added a detection reagent that is detectably labeled and recognizes the bound TCC. The detectable label can be, e.g., a fluorescent label or an enzymatic label. The assay results are expressed in CH50 unit equivalents per milliliter (CH50 U Eq/mL). Inhibition, e.g., as it pertains to terminal complement activity, includes at least an about 5 (e.g., at least an about 6, about 7, about 8, about 9, about 10, about 15, about 20, about 25, about 30, about 35, about 40, about 45, about 50, about 55, about or 60) % decrease in the activity of terminal complement in, e.g., a hemolytic assay or CH50eq assay as compared to the effect of a control antibody (or antigen-binding fragment thereof) under similar conditions and at an equimolar concentration. Substantial inhibition, as used herein, refers to inhibition of a given activity (e.g., terminal complement activity) of at least about 40% (e.g., at least about 45, about 50, about 55, about 60, about 65, about 70, about 75, about 80, about 85, about 90, about 95, or up to about 100%).

Complement functional tests can be used to monitor eculizumab treatment in VHF patients. See, e.g., Cugno et al., J Thromb Haemost 2014; 12: 1440-8. These tests include, for example, Wieslab (Wieslab complement System, Eurodiagnostica, MalmÖ, Sweden) for the classical, alternative, and mannose-binding complement pathways. See Id. for details of these assays.

There are known biomarkers (i.e., disease activity markers) associated with complement related conditions that can be monitored when treating patients suffering from a hemorrhagic fever virus infection with complement inhibitors. Lactate dehydrogenase, free hemoglobin, platelet counts, haptoglobin level, creatine serum levels can all be used to monitor a patient's therapy while undergoing treatment.

Lactate Dehydrogenase (LDH) is a marker of intravascular hemolysis and would be a useful indicator for monitoring treatment of patients with hemorrhagic fever virus infections. Hill, A. et al., Br. J. Haematol., 149:414-25, 2010; Hillmen, P. et al., N. Engl. J. Med., 350:552-9, 2004; Parker, C. et al., Blood, 106:3699-709, 2005. Red blood cells (RBCs) contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):359 Abs.0903, 2008; Kanakura, Y. et al., Int. J. Hematol., 93:36-46, 2011). Therefore LDH levels may be used to monitor the effect of treating patients with hemorrhagic fever virus infections using with C5 inhibitors such as anti-C5 antibodies.

LDH concentration can be measured, for example, in various samples obtained from a patient, in particular, serum samples. As used herein, the term “sample” refers to biological material from a subject. Although serum LDH concentration is of most interest, samples can be derived from other sources, including, for example, single cells, multiple cells, tissues, tumors, biological fluids, biological molecules or supernatants or extracts of any of the foregoing. The sample used will vary based on the assay format, the detection method and the nature of the tissues, cells or extracts to be assayed. Methods for preparing samples are known in the art and can be readily adapted to obtain a sample that is compatible with the method utilized.

In some embodiments, the C5 inhibitor can be administered to a patient in an amount and with a frequency that are effective to maintain serum LDH levels at within at least about 20 (e.g., about 19, about 18, about 17, about 16, about 15, about 14, about 13, about 12, about 11, about 10, about 9, about 8, about 7, about 6, or about 5) % of the normal range for LDH. See Hill et al. (2005) Blood 106(7):2559. In some embodiments, the complement inhibitor is administered to the patient in an amount and with a frequency that are effective to maintain a serum LDH level less than about 550 (e.g., less than about 540, about 530, about 520, about 510, about 500, about 490, about 480, about 470, about 460, about 450, about 440, about 430, about 420, about 410, about 400, about 390, about 380, about 370, about 360, about 350, about 340, about 330, about 320, about 310, about 300, about 290, about 280, or less than about 270) IU/L. To maintain systemic complement inhibition in a patient, the C5 inhibitor can be chronically administered to the patient, e.g., once a week, once every two weeks, twice a week, once a day, once a month, or once every three weeks.

In addition to the use of LDH as a biomarker described above, laboratory tests can be performed to determine whether a human subject suffering from a hemorrhagic fever virus infection has other complement related symptoms such as thrombocytopenia, microangiopathic hemolytic anemia, or acute renal insufficiency. Identification of these conditions can then be monitored for signs of improvement upon treatment with C5 complement inhibitors such as, for example, eculizumab or other anti-C5 antibodies. Thrombocytopenia can be diagnosed by a medical professional as one or more of: (i) a platelet count that is less than about 150,000/mm³ (e.g., less than about 60,000/mm³); (ii) a reduction in platelet survival time that is reduced, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than about 10 mg/dL (e.g., less than about 6.5 mg/dL); (ii) increased serum lactate dehydrogenase (LDH) concentrations (greater than about 460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668).

Methods of Treating Sepsis in a Patient

In certain aspects, the complement mediated disorder is sepsis.

In certain embodiments, a method is provided of treating sepsis in a patient, comprising determining that the serum level of LDH is elevated in the patient, and administering an effective amount of a C5 inhibitor to the patient. The serum LDH level can be considered elevated, for example, if it is a level that can be harmful to the patient, or, for another example, a level that is elevated compared to the normal level of serum LDH in that patient, or normal level for a patient based on size, age, etc. Normal level of serum LDH in a patient can mean a level that is not harmful to the patient, or, for another example, a level that is not elevated compared to the normal level of serum LDH in that patient, or normal level for a patient based on size, age, etc. In certain embodiments, serum LDH level is considered elevated if it is at or above about 570 U/L (in certain embodiments, at or above about 656 U/L). In certain embodiments, serum LDH level is considered normal if it is at or below about 450 U/L (in certain embodiments, at or below about 369 U/L). In certain embodiments, the method further comprises the additional step of determining that the level of serum LDH is reduced in the patient after administering the C5 inhibitor to the patient. The level of serum LDH can be determined by any method known in the art. In certain embodiments, the step of determining whether the level of serum LDH is elevated in a patient can be skipped. In certain embodiments, the patient is a human patient.

The disclosed method is for treating sepsis or septic shock (the term “sepsis” used herein includes “septic shock”) without regard to the origin, i.e., without regard for the type of infection. Any and all types of infection leading to sepsis are included.

Sepsis may be diagnosed by any means, including methods known in the art, or may be suspected.

In certain embodiments, a typical therapeutic treatment includes a series of doses, which will usually be administered concurrently with the monitoring of clinical endpoints with the dosage levels adjusted as needed to achieve the desired clinical outcome. In certain embodiments, a typical therapeutic treatment includes one or more dosages administered within about 12-48 hours after diagnosis of sepsis, possibly with follow-up dosages after that time period. In certain embodiments, treatment is administered in multiple dosages over at least a few hours or a few days. In certain embodiments, treatment is administered in multiple dosages over at least a week. In certain embodiments, treatment is administered in multiple dosages over at least a month. In certain embodiments, treatment is administered in multiple dosages over at least a year. In certain embodiments, treatment is administered in multiple dosages over the remainder of the patient's life.

The frequency of administration can also be adjusted according to various parameters. These include, for example, the clinical response, the plasma half-life of the C5 inhibitor, and the levels of the C5 inhibitor (such as an antibody) in a body fluid, such as, blood, plasma, serum, or synovial fluid. To guide adjustment of the frequency of administration, levels of the C5 inhibitor in the body fluid can be monitored during the course of treatment.

In certain embodiments, the dosage(s) and frequency of administration are determined according to the need of the patient, at the discretion of the treating physician.

For the treatment of sepsis by systemic administration of a C5 inhibitor, such as, for example, a polypeptide, administration of a large initial dose can be performed. Such a large initial dose can be followed by regularly repeated administration of tapered doses as needed. In other embodiments, the initial dose is given by both local and systemic routes, followed by repeated systemic administration of tapered doses.

In certain embodiments, a sepsis patient receives about 900 milligrams (mg) or about 1200 mg of eculizumab each week for the first 3 weeks, followed by an about 1200 mg dose on weeks 4, 6, and 8. After an initial 8-week eculizumab treatment period, the patient can optionally receive further treatment with eculizumab at about 1200 mg every other week, up to an additional 8 weeks.

In certain embodiments, a sepsis patient receives about 1200 milligrams (mg) of eculizumab each week for the first 8 weeks. After an initial 8-week eculizumab treatment period, the patient can optionally receive further treatment with eculizumab at about 1200 mg every other week, up to an additional 8 weeks.

In certain embodiments, a therapeutically effective amount of a C5 inhibitor (such as eculizumab) can include an amount (or various amounts in the case of multiple administrations) that improves the patient's chance of survival. In certain embodiments, a disclosed method improves the life expectancy of a patient by any amount of time, including at least one day, at least one week, at least two weeks, at least three weeks, at least one month, at least two months, at least three months, at least 6 months, at least one year, at least 18 months, at least two years, at least 30 months, or at least three years, or the duration of treatment.

In certain embodiments, a therapeutically effective amount of a C5 inhibitor (such as eculizumab) can include an amount (or various amounts in the case of multiple administrations) that reduces C5a levels, reduces serum LDH levels, reduces C-reactive protein level, reduces procalcitonin level, reduces serum amyloid A level, reduces mannan and/or antimannan antibody levels, reduces interferon-γ-inducible protein 10 (“IP-10”) level, results in the patient having little to no organ failure, reduces levels of one or more of lactic acid, serum glutamic oxaloacetic transaminase (“SGOT”), creatine kinase, and creatine, increases levels of one or more of platelets and plasma bicarbonate level, decreases levels of one or more of the proinflammatory cytokines that are over-produced, or reduces other symptoms of the disease, or any combination thereof. These parameters can be ascertained or measured by any methods known in the art. Exemplary methods are disclosed above.

There are known biomarkers (i.e., disease activity markers) associated with complement related conditions that can be monitored when treating patients suffering from sepsis with complement inhibitors that inhibit the formation of TCC. Lactate dehydrogenase, free hemoglobin, platelet counts, haptoglobin level, creatine serum levels, can all be used to monitor a patient's therapy while undergoing treatment. There are also biomarkers that can be used to monitor sepsis, such as, for example, C-reactive protein level, procalcitonin level, serum amyloid A level, mannan and/or antimannan antibody levels, or interferon-γ-inducible protein 10 (“IP-10”), or any combination thereof.

LDH is a marker of intravascular hemolysis and would be a useful indicator for monitoring treatment of patients with sepsis. Hill, A. et al., Br. J. Haematol., 149:414-25, 2010; Hillmen, P. et al., N. Engl. J. Med., 350:552-9, 2004; Parker, C. et al., Blood, 106:3699-709, 2005. Red blood cells (RBCs) contain large amounts of LDH, and a correlation between cell-free hemoglobin and LDH concentration has been reported in vitro (Van Lente, F. et al., Clin. Chem., 27:1453-5, 1981) and in vivo (Kato, G. et al., Blood, 107:2279-85, 2006). The consequences of hemolysis are independent of anemia (Hill, A. et al., Haematologica, 93(s1):359 Abs.0903, 2008; Kanakura, Y. et al., Int. J. Hematol., 93:36-46, 2011). Therefore LDH levels may be used to monitor the effect of treating patients with sepsis with C5 inhibitors such as anti-C5 antibodies. As discussed above, in certain embodiments, the invention provides a method of treating sepsis in a patient, comprising determining that the serum level of LDH is elevated in the patient, and administering an effective amount of a C5 inhibitor to the patient. Serum LDH levels have been observed to be elevated in patients with sepsis. Zein et al., Chest, 2004; 126(4_meetingAbstracts):873S. doi:10.1378/chest.126.4_MeetingAbstracts.873S.

LDH concentration can be measured, for example, in various samples obtained from a patient, in particular, serum samples. As used herein, the term “sample” refers to biological material from a subject. Although serum LDH concentration is of most interest, samples can be derived from other sources, including, for example, single cells, multiple cells, tissues, tumors, biological fluids, biological molecules or supernatants or extracts of any of the foregoing. The sample used will vary based on the assay format, the detection method and the nature of the tissues, cells or extracts to be assayed. Methods for preparing samples are known in the art and can be readily adapted to obtain a sample that is compatible with the method utilized.

In some embodiments, the complement inhibitor is administered to the patient in an amount and with a frequency that are effective to maintain a serum LDH level at or below about 450 U/L. To maintain systemic complement inhibition in a patient, the C5 inhibitor can be chronically administered to the patient, e.g., once a week, once every two weeks, twice a week, once a day, once a month, or once every three weeks.

In addition to the use of LDH as a biomarker described above, laboratory tests can be performed to determine whether a human subject suffering from sepsis has other complement related symptoms such as thrombocytopenia, microangiopathic hemolytic anemia, or acute renal insufficiency. Identification of these conditions can then be monitored for signs of improvement upon treatment with C5 complement inhibitors such as, for example, eculizumab or other anti-C5 antibodies. Thrombocytopenia can be diagnosed by a medical professional as one or more of: (i) a platelet count that is less than about 150,000/mm³ (e.g., less than about 60,000/mm³); (ii) a reduction in platelet survival time that is reduced, reflecting enhanced platelet disruption in the circulation; and (iii) giant platelets observed in a peripheral smear, which is consistent with secondary activation of thrombocytopoiesis. Microangiopathic hemolytic anemia can be diagnosed by a medical professional as one or more of: (i) hemoglobin concentrations that are less than about 10 mg/dL (e.g., less than about 6.5 mg/dL); (ii) increased serum lactate dehydrogenase (LDH) concentrations (greater than about 460 U/L); (iii) hyperbilirubinemia, reticulocytosis, circulating free hemoglobin, and low or undetectable haptoglobin concentrations; and (iv) the detection of fragmented red blood cells (schistocytes) with the typical aspect of burr or helmet cells in the peripheral smear together with a negative Coombs test. See, e.g., Kaplan et al. (1992) “Hemolytic Uremic Syndrome and Thrombotic Thrombocytopenic Purpura,” Informa Health Care (ISBN 0824786637) and Zipfel (2005) “Complement and Kidney Disease,” Springer (ISBN 3764371668).

C-reactive protein C (“CRP”) level can rise up to 1000-fold in the blood in response to inflammation and infection. Chan and Gu, Expert Rev Mol Diagn. 2011; 11(5): 487-496. Procalcitonin (“PCT”) level also rises by up to 1000-fold under inflammatory conditions in a bacterial infection. Id. Serum amyloid A (“SAA”) is expressed at levels up to 1000-times higher after 8-24 hours from the onset of sepsis. Id. Mannan and antimannan antibody levels can become elevated in response to fungal infections. Id. Interferon-γ-inducible protein 10 (“IP-10”) level can be elevated in serum of patients with viral infections. Id. Reduction in one or more of these protein levels can be monitored in a patient with sepsis for improvement of the disease. Assays for these proteins are known in the art. For example, assays for CRP include the IMx system (Abbott Laboratories, IL, USA) and the BN II analyzer (Dade Behring, IL, USA); assays for PCT include the LUMItest—immunoluminometric assay and the LUMItest (both by BRAHMS, Thermofisher, Berlin, Germany); assays for SAA include the BN II analyzer and the BN ProSpec analyzer (both by Dade Behring, IL, USA); and assays for Mannan and antimannan antibody levels are available from Bio-TRad Laboratories, CA, USA. See, e.g., Chan and Gu, Expert Rev Mol Diagn. 2011; 11(5): 487-496.

Methods of Treating STEC-HUS in a Patient

In another aspect, a method is provided to treat STEC-HUS, a complement mediated disorder caused by an infectious agent, in a patient.

STEC-HUS can be diagnosed by methods known in the art. Symptoms of STEC-HUS include, but are not limited to, organ (such as kidney) failure, systemic thrombotic microangiopathy (TMA), neurological symptoms, elevated LDH level, elevated platelet count, and elevated hemoglobin level. Symptoms of STEC infection can include, for example, stomach cramp and bloody diarrhea, and may include mild fever and vomiting. Symptoms of HUS can include, for example, decreased urination, swelling of limbs, high blood pressure, jaundice (yellowish discoloration of the skin and the whites of the eyes), epileptic seizures (fits) or other neurological symptoms, bleeding into the skin, and lethargy. STEC infection can be detected by laboratory testing of a patients' stool sample.

STEC and HUS may be contracted by, for example, eating undercooked beef, in particular ground or minced beef, drinking unpasteurized milk, drinking contaminated water, close contact with a person who has the bacteria in their feces, contact with farm animals, particularly sheep and cattle and their feces, and eating fresh produce contaminated with animal feces.

STEC-HUS's time course can be: about 3 days after ingesting STEC-contaminated material, individuals develop moderate diarrhea and significant abdominal pain. About 3 days later, bloody diarrhea develops in most of these individuals prompting medical attention. A stool sample is taken for analysis of STEC and Shiga toxin. It is during the hemorrhagic colitis stage that Stx1 and/or Stx2 enter the blood circulation setting in action a series of toxemic reactions that culminate in renal failure in 5-15% of the patients. STEC does not colonize the blood, thus D+HUS is a toxemic rather than a bacteremic event. 4 days after the hemorrhagic colitis phase, the toxemic period advances to acute renal failure. Most patients resolve the systemic complications and do not progress to renal failure. Although the latter 4 days represent a potential ‘therapeutic window’, there is no therapeutic treatment other than fluid volume control and dialysis currently available to reduce or prevent renal failure in STEC-HUS. Another complicating factor in STEC-HUS is that antibiotics are not recommended in the earlier phases, i.e., prior to appearance of bloody diarrhea because STEC bacteria respond to some antibiotics by producing excess Shiga toxin.

The timeline can be as follows: Incubation time: The time from eating the contaminated food to the beginning of symptoms. For E. coli O157, this is typically 3-4 days. Time to treatment: The time from the first symptom until the person seeks medical care, when a diarrhea sample is collected for laboratory testing. This time lag may be 1-5 days. Time to diagnosis: The time from when a person gives a sample to when E. coli O157 is obtained from it in a laboratory. This may be 1-3 days from the time the sample is received in the laboratory. Sample shipping time: The time required to ship the E. coli O157 bacteria from the laboratory to the state public health authorities that will perform “DNA fingerprinting”. This may take 0-7 days depending on transportation arrangements within a state and the distance between the clinical laboratory and public health department. Time to “DNA fingerprinting”: The time required for the state public health authorities to perform “DNA fingerprinting” on the E. coli O157 and compare it with the outbreak pattern. Ideally this can be accomplished in 1 day. However, many public health laboratories have limited staff and space, and experience multiple emergencies at the same time. Thus, the process may take 1-4 days. The time from the beginning of the patient's illness to the confirmation that he or she was part of an outbreak is typically about 2-3 weeks. Case counts in the midst of an outbreak investigation must be interpreted within this context.

This disclosure provides a method of treating a human patient with Shiga toxin-producing E. coli hemolytic uremic syndrome (STEC-HUS), the method comprising administering to the patient an effective amount of an anti-C5 antibody, or antigen binding fragment thereof, wherein the method comprises an administration cycle comprising an induction phase followed by a maintenance phase, wherein:
the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or
the anti-C5 antibody, or antigen binding fragment thereof, is administered during the induction phase at a dose of 600 mg weekly for 1 week, starting at day 0, and is administered during the maintenance phase at a dose of 600 mg every week; or
the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

In certain embodiments, the anti-C5 antibody, or antigen binding fragment thereof, is administered during the induction phase at a dose of 900 mg weekly for 4 weeks, starting at day 0, and is administered during the maintenance phase at a dose of 1200 mg in week 5 (day 28) and then 1200 mg every two weeks, wherein the human patient is greater than or equal to 40 kg.

In certain embodiments, the anti-C5 antibody, or antigen binding fragment thereof, is administered during the induction phase at a dose of 600 mg weekly for 2 weeks, starting at day 0, and is administered during the maintenance phase at a dose of 900 mg in week 3 (day 14), and then 900 mg every two weeks, wherein the human patient is between 30 kg and 40 kg.

In certain embodiments, the anti-C5 antibody, or antigen binding fragment thereof, is administered during the induction phase at a dose of 600 mg weekly for 2 weeks, starting at day 0, and is administered during the maintenance phase at a dose of 600 mg in week 3 (day 14), and then 600 mg every two weeks, wherein the human patient is between 20 kg and 30 kg.

In certain embodiments, the anti-C5 antibody, or antigen binding fragment thereof, is administered during the induction phase at a dose of 600 mg weekly for 1 week, starting at day 0, and is administered during the maintenance phase at a dose of 600 mg every week (starting from day 7), wherein the human patient is between 10 kg and 20 kg.

In certain embodiments, the anti-C5 antibody, or antigen binding fragment thereof, is administered during the induction phase at a dose of 300 mg weekly for 1 week, starting at day 0, and is administered during the maintenance phase at a dose of 300 mg at week 2 (day 7) and then every 3 weeks, wherein the human patient is between 5 kg and 10 kg.

In some embodiments, the treatment method maintains a serum trough concentration of the anti-C5 antibody, or antigen binding fragment thereof, of about 35 μg/mL to about 700 μg/mL during the induction phase and/or the maintenance phase.

The anti-C5 antibody, or antigen binding fragment thereof, may be formulated for intravenous administration, including administration as an IV infusion. In some embodiments, the patient has not previously been treated with a complement inhibitor. The administration cycle can be 8 weeks; or it can be 16 weeks.

Patients treated according to the methods disclosed herein preferably experience improvement in at least one sign of STEC-HUS. For example, the treatment may produce at least one therapeutic effect selected from the group consisting of reduced systemic thrombotic microangiopathy (TMA), improved renal function, improved platelet count toward normal level, improvement in hemoglobin level toward normal level, improvement in LDH level toward normal level, and neurological improvement. In other embodiments, the improvement of clinical benefit rate is about 20% 20%, 30%, 40%, 50%, 60%, 70%, 80% or more compared to no treatment. In some embodiments, the treatment allows the patient to live longer, by at least 1 day. In some embodiments, the treatment results in terminal complement inhibition.

In certain embodiments, the treatment is safe and well tolerated.

In certain embodiments, the treatment results in improvement in systemic TMA and vital organ involvement in at least 80% of patients by week 8. In certain embodiments, the treatment results in improvement in systemic TMA and vital organ involvement in at least 90% of patients by week 28.

In certain embodiments, the treatment results in normalization relative to baseline of the hematologic parameters of platelet count, hemoglobin and LDH in at least 90% of patients in 20 days.

In certain embodiments, the treatment results in improvements relative to baseline in renal function as assessed by serum creatinine. In some embodiments, the treatment results in improvements relative to baseline in eGFR in patients not on dialysis at baseline. In certain embodiments, the treatment results in an increase in eGFR from baseline of greater than or equal to 15 mL/min/1.73 m³ by day 56 in patients not on dialysis at baseline. In certain embodiments, the treatment results in an increase in eGFR from baseline of greater than or equal to 60 mL/min/1.73 m³ in at least 70% of all patients by week 28. In some embodiments, the treatment results in an increase in eGFR from baseline of greater than or equal to 90 mL/min/1.73 m³ in at least 25% of all patients by week 28. In certain embodiments, the treatment results in discontinuation of dialysis by week 28 in at least 90% of patients on dialysis at baseline.

In certain embodiments, the treatment results in improvements relative to baseline in neurological function as measured by Modified Rankin Score (MRS) in patients with baseline neurological involvement. In certain embodiments, the treatment results in achieving essentially normal neurological function with no persistent deficit in at least 90% of patients by week 28. In certain embodiments, the treatment results in all patients achieving seizure free status by Week 28.

Also provided herein are kits which include a pharmaceutical composition containing an anti-C5 antibody, or antigen binding fragment thereof, such as an eculizumab variant (including those disclosed herein) or eculizumab, and a pharmaceutically-acceptable carrier, in a therapeutically effective amount adapted for use in the methods disclosed herein. The kits optionally also can include instructions, e.g., comprising administration schedules, to allow a practitioner (e.g., a physician, nurse, or patient) to administer the composition contained therein to administer the composition to a patient having STEC-HUS. The kit also can include a syringe.

Optionally, the kits include multiple packages of the single-dose pharmaceutical compositions, each containing an effective amount of the anti-C5 antibody, or antigen binding fragment thereof, for a single administration in accordance with the methods provided herein. Instruments or devices necessary for administering the pharmaceutical composition(s) also may be included in the kits. For instance, a kit may provide one or more pre-filled syringes containing an amount of the anti-C5 antibody, or antigen binding fragment thereof.

C5 Inhibitor

A C5 inhibitor (an inhibitor of complement C5 protein) for use in a method or a kit disclosed herein can be any C5 inhibitor. In certain embodiments, the C5 inhibitor for use in methods and kits disclosed herein is a polypeptide inhibitor. In certain embodiments, the C5 inhibitor is eculizumab, an antigen-binding fragment thereof, a polypeptide comprising the antigen-binding fragment of eculizumab, a fusion protein comprising the antigen binding fragment of eculizumab, or a single chain antibody version of eculizumab, or a small-molecule C5 inhibitor.

In some embodiments, the complement C5 protein is a human complement C5 protein (the human proprotein is depicted in SEQ ID NO:4). In other embodiments, the complement C5 protein is a non-human animal complement C5 protein, including other primate complement C5 protein and other mammalian complement C5 protein.

In some embodiments, the C5 inhibitor is a small-molecule chemical compound. One example of a small molecule chemical compound that is a C5 inhibitor is Aurin tricarboxylic acid. In other embodiments, the C5 inhibitor is a polypeptide.

The C5 inhibitor is one that binds to a complement C5 protein and is also capable of inhibiting the generation of C5a. A C5-binding inhibitor can also be capable of inhibiting, e.g., the cleavage of C5 to fragments C5a and C5b, and thus preventing the formation of terminal complement complex.

For example, an anti-C5 antibody blocks the generation or activity of the C5a active fragment of a C5 protein (e.g., a human C5 protein). Through this blocking effect, the antibody inhibits, e.g., the proinflammatory effects of C5a. An anti-C5 antibody can further have activity in blocking the generation or activity of C5b. Through this blocking effect, the antibody can further inhibit, e.g., the generation of the C5b-9 membrane attack complex at the surface of a cell.

In some embodiments, the C5 inhibitor is a polypeptide inhibitor. In yet further other embodiments, the polypeptide inhibitor is eculizumab. SEQ ID NO:5 depicts the entire heavy chain of eculizumab; SEQ ID NO:6 depicts the entire light chain of eculizumab; SEQ ID NOs:9-11 depict, respectively, CDR1-3 of the heavy chain of eculizumab; SEQ ID NOs:12-14 depict, respectively, CDR1-3 of the light chain of eculizumab; SEQ ID NO:15 depicts the variable region of the heavy chain of eculizumab; and SEQ ID NO:16 depicts the variable region of the light chain of Eculizumab. Eculizumab is a humanized anti-human C5 monoclonal antibody (Alexion Pharmaceuticals, Inc.), with a human IgG2/IgG4 hybrid constant region, so as to reduce the potential to elicit proinflammatory responses. Eculizumab has the trade name Soliris® and is currently approved for treating paroxysmal nocturnal hemoglobinuria (“PNH”) and atypical hemolytic uremic syndrome (“aHUS”). Paroxysmal nocturnal hemoglobinuria is a form of hemolytic anemia, intravascular hemolysis being a prominent feature due to the absence of the complement regulatory protein CD59 and CD55. CD59, for example, functions to block the formation of the terminal complement complex. AHUS involves chronic uncontrolled complement activation, resulting in, inter alia, inhibition of thrombolitic microangiopathy, the formation of blood clots in small blood vessels throughout the body, and acute renal failure. Eculizumab specifically binds to human C5 protein and blocks the formation of the generation of the potent proinflammatory protein C5a. Eculizumab further blocks the formation of the terminal complement complex. Eculizumab treatment reduces intravascular hemolysis in patients with PNH and decreases complement levels in aHUS. See, e.g., Hillmen et al., N Engl J Med 2004; 350:552-9; Rother et al., Nature Biotechnology 2007; 25(11): 1256-1264; Hillmen et al., N Engl J Med 2006, 355;12, 1233-1243; Zuber et al., Nature Reviews Nephrology 8, 643-657 (2012) doi:10.1038/nrneph.2012.214; U.S. Patent Publication Number 2012/0237515, and U.S. Pat. No. 6,355,245.

In yet further other embodiments, the C5 inhibitor is a single chain version of eculizumab, including pexelizumab (SEQ ID NO:1)—a specific single chain version of the whole antibody eculizumab. See, e.g., Whiss (2002) Curr Opin Investig Drugs 3(6):870-7; Patel et al. (2005) Drugs Today (Barc) 41(3):165-70; Thomas et al. (1996) Mol Immunol 33(17-18):1389-401; and U.S. Pat. No. 6,355,245. In yet other embodiments, the inhibitor for use in methods of this invention is a single chain variant of pexelizumab, with the arginine (R) at position 38 (according to Kabat numbering and the amino acid sequence number set forth in SEQ ID NO:2) of the light chain of the pexelizumab antibody amino acid sequence changed to a glutamine (Q). The single chain antibody having the amino acid sequence depicted in SEQ ID NO:2 is a variant of the single chain antibody pexelizumab (SEQ ID NO:1), in which the arginine (R) at position 38 has been substituted with a glutamine (Q). An exemplary linker amino acid sequence present in a variant pexelizumab antibody is shown in SEQ ID NO:3.

In certain embodiments, the anti-C5 antibody is a variant derived from eculizumab, having one or more improved properties (e.g., improved pharmacokinetic properties) relative to eculizumab. The variant eculizumab antibody (also referred to herein as an eculizumab variant, a variant eculizumab, or the like) or C5-binding fragment thereof is one that: (a) binds to complement component C5; (b) inhibits the generation of C5a; and can further inhibit the cleavage of C5 into fragments C5a and C5b. The variant eculizumab antibody can have a serum half-life in a human that is greater than, or at least, 10 (e.g., greater than, or at least, 11, 12, 13, 14, 15, 16, 17, 18, 19, 20, 21, 22, 23, 24, 25, 26, 27, 28, 29, 30, 31, 32, 33 or 34) days. Such variant eculizumab antibodies are described in PCT/US2015/019225 and U.S. Pat. No. 9,079,949.

In certain embodiments, the eculizumab variant antibody is an antibody defined by the sequences depicted in SEQ ID NO:7 (heavy chain) and SEQ ID NO:8 (light chain), or an antigen-binding fragment thereof. This antibody binds to human C5 and inhibits the formation of C5a, as well as the cleavage of C5 to fragments C5a and C5b, and thus preventing the formation of terminal complement complex.

In certain embodiments, the eculizumab variant is BNJ441 (an antibody comprising the sequences depicted in SEQ ID NO:24, SEQ ID NO:25, and SEQ ID NO:16; see also the sequences depicted in SEQ ID NOs:6-8). In certain embodiments, the eculizumab variant is defined by the sequences depicted in SEQ ID NO:24, SEQ ID NO:25 and SEQ ID NO:8.

In certain embodiments, the C5 inhibitor is a polypeptide C5 inhibitor comprising or consisting of one or more sequences depicted by SEQ ID NOs:1-3, 5-16, and 23-29, and 33, such that the resulting polypeptide binds to complement protein C5 (“C5”).

In some embodiments, a C5-binding polypeptide for use in methods of this disclosure is not a whole antibody. In some embodiments, a C5-binding polypeptide is a single chain antibody. In some embodiments, a C5-binding polypeptide for use in methods of this disclosure is a bispecific antibody. In some embodiments, a C5-binding polypeptide for use in methods of this disclosure is a humanized monoclonal antibody, a chimeric monoclonal antibody, or a human monoclonal antibody, or an antigen binding fragment of any of them.

The C5-binding polypeptide for use in methods of this disclosure can comprise, or can consist of, the amino acid sequence depicted in SEQ ID NO:1, SEQ ID NO:2, SEQ ID NO:5 and SEQ ID NO:6, or SEQ ID NO: 7 and SEQ ID NO: 8, or an antigen binding fragment of any of the above. The polypeptide can comprise one or more of the amino acid sequence depicted in SEQ ID NOs:9-16.

In yet other embodiments, the C5 inhibitor is LFG316 (Novartis, Basel, Switzerland, and MorphoSys, Planegg, Germany) or another antibody defined by the sequences of Table 1 in U.S. Pat. No. 8,241,628 and U.S. Pat. No. 8,883,158, ARC1905 (Ophthotech, Princeton, N.J. and New York, N.Y.), which is an anti-C5 pegylated RNA aptamer (see, e.g., Keefe et al., Nature Reviews Drug Discovery 9, 537-550 (July 2010) doi:10.1038/nrd3141), Mubodina® (Adienne Pharma & Biotech, Bergamo, Italy) (see, e.g., U.S. Pat. No. 7,999,081), rEV576 (coversin) (Volution Immuno-pharmaceuticals, Geneva, Switzerland) (see, e.g., Penabad et al., Lupus, 2014 October; 23(12):1324-6. doi: 10.1177/0961203314546022.), ARC1005 (Novo Nordisk, Bagsvaerd, Denmark), SOMAmers (SomaLogic, Boulder, Colo.), SOB1002 (Swedish Orphan Biovitrum, Stockholm, Sweden), RA101348 (Ra Pharmaceuticals, Cambridge, Mass.), Aurin Tricarboxylic Acid (“ATA”), and anti-C5-siRNA (Alnylam Pharmaceuticals, Cambridge, Mass.), and Ornithodoros moubata C inhibitor (‘OmCI”).

In some embodiments, the polypeptide C5 inhibitor is an antibody (referred to herein as an “anti-C5 antibody,” C-5 binding antibody, or the like), or an antigen binding fragment thereof. The antibody can be a monoclonal antibody. In other embodiments, the polypeptide C5 inhibitor comprises the variable region, or a fragment thereof, of an antibody, such as a monoclonal antibody. In other embodiments, the polypeptide C5 inhibitor is an immunoglobulin that binds specifically to a C5 complement protein. In other embodiments, the polypeptide inhibitor is an engineered protein or a recombinant protein, as defined hereinabove. In some embodiments, a C5-binding polypeptide is not a whole antibody, but comprises parts of an antibody. In some embodiments, a C5-binding polypeptide is a single chain antibody. In some embodiments, a C5-binding polypeptide is a bispecific antibody. In some embodiments, the C5-binding polypeptide is a humanized monoclonal antibody, a chimeric monoclonal antibody, or a human monoclonal antibody, or an antigen binding fragment of any of them. Methods of making a polypeptide C5 inhibitor, including antibodies, are known in the art.

As stated above, the C5 inhibitor, including a C5-binding polypeptides, can inhibit complement component C5. In particular, the inhibitors, including polypeptides, inhibit the generation of the C5a anaphylatoxin, or the generation of c5a and the C5b active fragments of a complement component C5 protein (e.g., a human C5 protein). Accordingly, the C5 inhibitors inhibit, e.g., the pro-inflammatory effects of C5a; and can inhibit the generation of the C5b-9 membrane attack complex (“MAC”) at the surface of a cell and subsequent cell lysis. See, e.g., Moongkarndi et al. (1982) Immunobiol 162:397 and Moongkarndi et al. (1983) Immunobiol 165:323.

Suitable methods for measuring inhibition of C5 cleavage are known in the art. For example, the concentration and/or physiologic activity of C5a and/or C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest 50(3):606-16 and Wurzner et al. (1991) Complement Inflamm 8:328-340). For C5b, hemolytic assays or assays for soluble C5b-9 known in the art can be used. Other assays known in the art can also be used.

For those C5 inhibitors that also inhibit TCC formation, inhibition of complement component C5 can also reduce the cell lysing ability of complement in a subject's body fluids. Such reductions of the cell-lysing ability of complement present can be measured by methods well known in the art such as, for example, by a conventional hemolytic assay such as the hemolysis assay described by Kabat and Mayer (eds), “Experimental Immunochemistry, 2^{nd} Edition,” 135-240, Springfield, Ill., C C Thomas (1961), pages 135-139, or a conventional variation of that assay such as the chicken erythrocyte hemolysis method as described in, e.g., Hillmen et al. (2004) N Engl J Med 350(6):552.

In some embodiments, the C5-binding polypeptides are variant antibodies of an anti-C5 antibody (such as eculizumab) that still bind to the antigen, including deletion variants, insertion variants, and/or substitution variants. See, e.g., the polypeptides depicted in SEQ ID NO:1, SEQ ID NO:2, or SEQ ID NO:7 and SEQ ID NO:8. Methods of making such variants, by, for example, recombinant DNA technology, are well known in the art.

In some embodiments, a C5-binding polypeptide is a fusion protein. The fusion protein can be constructed recombinantly such that the fusion protein is expressed from a nucleic acid that encodes the fusion protein. The fusion protein can comprise one or more C5-binding polypeptide segments (e.g., C5-binding segments depicted in SEQ ID NO:1, SEQ ID NO:2, or SEQ ID NO:5 and/or SEQ ID NO:6, SEQ ID NO: 7 and/or SEQ ID NO: 8, or any one or more of SEQ ID NOs:9-16) and one or more segments that are heterologous to the C5-binding segment(s). The heterologous sequence can be any suitable sequence, such as, for example, an antigenic tag (e.g., FLAG, polyhistidine, hemagglutinin (“HA”), glutathione-S-transferase (“GST”), or maltose-binding protein (“MBP”)). Heterologous sequences can also be proteins useful as diagnostic or detectable markers, for example, luciferase, green fluorescent protein (“GFP”), or chloramphenicol acetyl transferase (“CAT”). In some embodiments, the heterologous sequence can be a targeting moiety that targets the C5-binding segment to a cell, tissue, or microenvironment of interest. In some embodiments, the targeting moiety is a soluble form of a human complement receptor (e.g., human complement receptor 2) or an antibody (e.g., a single chain antibody) that binds to C3b or C3d. In some embodiments, the targeting moiety is an antibody that binds to a tissue-specific antigen, such as a kidney-specific antigen. Methods of constructing such fusion proteins, such as by recombinant DNA technology, are well known in the art.

In some embodiments, the C5-binding polypeptides are fused to a targeting moiety. For example, a construct can contain a C5-binding polypeptide and a targeting moiety that targets the polypeptide to a site of complement activation. Such targeting moieties can include, e.g., soluble form of complement receptor 1 (CR1), a soluble form of complement receptor 2 (CR2), or an antibody (or antigen-binding fragment thereof) that binds to C3b and/or C3d.

Methods for generating fusion proteins (e.g., fusion proteins containing a C5-binding polypeptide and a soluble form of human CR1 or human CR2), including recombinant DNA technology, are known in the art and described in, e.g., U.S. Pat. No. 6,897,290; U.S. patent application publication no. 2005265995; and Song et al. (2003) J Clin Invest 11(12):1875-1885.

In certain embodiments, the C5 inhibitor is a bispecific antibody. Methods for producing a bispecific antibody (e.g., a bispecific antibody comprising an anti-C5 antibody and an antibody that binds to C3b and/or C3d) are also known in the art. A bispecific antibody comprising a C5-binding antibody and any other antibody is contemplated.

A wide variety of bispecific antibody formats are known in the art of antibody engineering and methods for making the bispecific antibodies (e.g., a bispecific antibody comprising an anti-C5 antibody [i.e., a C5-binding antibody] and an antibody that binds to C3b, C3d, or a tissue-specific antigen) are well within the purview of those skilled in the art. See, e.g., Suresh et al. (1986) Methods in Enzymology 121:210; PCT Publication No. WO 96/27011; Brennan et al. (1985) Science 229:81; Shalaby et al., J. Exp. Med. (1992) 175:217-225; Kostelny et al. (1992) J Immunol 148(5):1547-1553; Hollinger et al. (1993) Proc Natl Acad Sci USA 90:6444-6448; Gruber et al. (1994) J Immunol 152:5368; and Tutt et al. (1991) J Immunol 147:60.

Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques. U.S. Pat. No. 5,534,254 describes several different types of bispecific antibodies including, e.g., single chain Fv fragments linked together by peptide couplers, chelating agents, or chemical or disulfide couplings. In another example, Segal and Bast [(1995) Curr Protocols Immunol Suppl. 14:2.13.1-2.13.16] describes methods for chemically cross-linking two monospecific antibodies to thus form a bispecific antibody. A bispecific antibody can be formed, e.g., by conjugating two single chain antibodies which are selected from, e.g., a C5-binding antibody and an antibody that binds to, e.g., C3b, C3d, or a lung-specific antigen, an eye-specific antigen, a kidney-specific antigen, etc.

The bispecific antibody can be a tandem single chain (sc) Fv fragment, which contains two different scFv fragments covalently tethered together by a linker (e.g., a polypeptide linker). See, e.g., Ren-Heidenreich et al. (2004) Cancer 100:1095-1103 and Korn et al. (2004) J Gene Med 6:642-651. Examples of linkers can include, but are not limited to, (Gly₄Ser)₂ [GGGGSGGGGS, SEQ ID NO:17], (Gly₄Ser)₃ [GGGGSGGGGSGGGGS, SEQ ID NO:18], (Gly₃Ser)₄ [GGGSGGGSGGGSGGGS, SEQ ID NO:19], (G₃S) [GGGS, SEQ ID NO:20], SerGly₄ [SGGGG, SEQ ID NO:21], and SerGly₄SerGly₄ [SGGGGSGGGG, SEQ ID NO:22].

In some embodiments, the linker can contain, or be, all or part of a heavy chain polypeptide constant region such as a CH1 domain as described in, e.g., Grosse-Hovest et al. (2004) Proc Natl Acad Sci USA 101:6858-6863. In some embodiments, the two antibody fragments can be covalently tethered together by way of a polyglycine-serine or polyserine-glycine linker as described in, e.g., U.S. Pat. Nos. 7,112,324 and 5,525,491, respectively. See also U.S. Pat. No. 5,258,498. Methods for generating bispecific tandem scFv antibodies are described in, e.g., Maletz et al. (2001) Int J Cancer 93:409-416; Hayden et al. (1994) Ther Immunol 1:3-15; and Honemann et al. (2004) Leukemia 18:636-644. Alternatively, the antibodies can be “linear antibodies” as described in, e.g., Zapata et al. (1995) Protein Eng. 8(10):1057-1062. Briefly, these antibodies comprise a pair of tandem Fd segments (V_{H}-C_{H}1-V_{H}-C_{H}1) that form a pair of antigen binding regions.

A bispecific antibody can also be a diabody. Diabody technology described by, e.g., Hollinger et al. (1993) Proc Natl Acad Sci USA 90:6444-6448 has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. See also Zhu et al. (1996) Biotechnology 14:192-196 and Helfrich et al. (1998) Int J Cancer 76:232-239. Bispecific single chain diabodies (“scDb”) as well as methods for generating scDb are described in, e.g., Brüsselbach et al. (1999) Tumor Targeting 4:115-123; Kipriyanov et al. (1999) J Mol Biol 293:41-56; and Nettlebeck et al. (2001) Mol Ther 3:882-891.

Variant forms of bispecific antibodies such as the tetravalent dual variable domain immunoglobulin (DVD-Ig) molecules described in Wu et al. (2007) Nat Biotechnol 25(11):1290-1297 can also be used in the methods of this invention. The DVD-Ig molecules are designed such that two different light chain variable domains (V_{L}) from two different parent antibodies are linked in tandem directly or via a short linker by recombinant DNA techniques, followed by the light chain constant domain. Methods for generating DVD-Ig molecules from two parent antibodies are further described in, e.g., PCT Publication Nos. WO 08/024188 and WO 07/024715. Also embraced is the bispecific format described in, e.g., U.S. patent application publication no. 20070004909. Another bispecific format that can be used is the Cross-Over Dual V Region (CODV-Ig) which is a format for engineering four domain antibody-like molecules described in WO2012/135345. CODV-Ig was shown to be useful in engineering bispecific antibody-like molecules where steric hindrance at the C-terminal V domains (internal) may prevent construction of a DVD-Ig.

The C5-binding antibodies and/or targeting-moieties that are used to form the bispecific antibody molecules can be, e.g., chimeric, humanized, rehumanized, deimmunized, or fully human, all of which are well known in the art.

C5 inhibitors that are small molecule chemical compounds can be produced by methods known in the art.

The C5-binding inhibitors, including polypeptides and antibodies, used in the methods of this invention can be produced using a variety of techniques known in the art of molecular biology and protein chemistry.

For example, a nucleic acid encoding a C5-binding polypeptide (e.g., a C5-binding polypeptide comprising or consisting of the amino acid sequence depicted in SEQ ID NO:2) can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

An exemplary nucleic acid, which encodes an exemplary C5-binding polypeptide (Pexelizumab), is as follows:

(SEQ ID NO: 1)
GATATCCAGATGACCCAGTCCCCGTCCTCCCTGTCCGCCTCTGTGGGCG
ATAGGGTCACCATCACCTGCGGCGCCAGCGAAAACATCTATGGCGCGCT
GAACTGGTATCAACAGAAACCCGGGAAAGCTCCGAAGCTTCTGATTTAC
GGTGCGACGAACCTGGCAGATGGAGTCCCTTCTCGCTTCTCTGGATCCG
GCTCCGGAACGGATTTCACTCTGACCATCAGCAGTCTGCAGCCTGAAGA
CTTCGCTACGTATTACTGTCAGAACGTTTTAAATACTCCGTTGACTTTC
GGACAGGGTACCAAGGTGGAAATAAAACGTACTGGCGGTGGTGGTTCTG
GTGGCGGTGGATCTGGTGGTGGCGGTTCTCAAGTCCAACTGGTGCAATC
CGGCGCCGAGGTCAAGAAGCCAGGGGCCTCAGTCAAAGTGTCCTGTAAA
GCTAGCGGCTATATTTTTTCTAATTATTGGATTCAATGGGTGCGTCAGG
CCCCCGGGCAGGGCCTGGAATGGATGGGTGAGATCTTACCGGGCTCTGG
TAGCACCGAATATACCGAAAATTTTAAAGACCGTGTTACTATGACGCGT
GACACTTCGACTAGTACAGTATACATGGAGCTCTCCAGCCTGCGATCGG
AGGACACGGCCGTCTATTATTGCGCGCGTTATTTTTTTGGTTCTAGCCC
GAATTGGTATTTTGATGTTTGGGGTCAAGGAACCCTGGTCACTGTCTCG
AGCTGA.

In some embodiments, the nucleic acid comprises nucleotides 1-738 of SEQ ID NO:1, e.g., in embodiments where carboxyl-terminal fusion proteins are to be generated or produced.

Several possible vector systems (such as plasmid vector systems) well known in the art are available for the expression of C5-binding polypeptides from nucleic acids in a number of cells, including in mammalian cells.

The expression vectors can be introduced by methods well known in the art into cells in a manner suitable for subsequent expression of the nucleic acid.

A C5-binding polypeptide can be expressed in any appropriate host cells. Appropriate host cells include, for example, yeast, bacteria, insect, plant, and mammalian cells, including bacteria such as E. coli, fungi such as Saccharomyces cerevisiae and Pichia pastoris, insect cells such as SF9, mammalian cell lines (e.g., human cell lines), primary cell lines (e.g., primary mammalian cells), Chinese hamster ovary (“CHO”) cells, and a suitable myeloma cell line such as NSO.

In some embodiments, a C5-binding polypeptide can be expressed in, and purified from, transgenic animals (e.g., transgenic mammals). For example, a C5-binding polypeptide can be produced in transgenic non-human mammals (e.g., rodents, sheep or goats) and isolated from milk as described in, e.g., Houdebine (2002) Curr Opin Biotechnol 13(6):625-629; van Kuik-Romeijn et al. (2000) Transgenic Res 9(2):155-159; and Pollock et al. (1999) J Immunol Methods 231(1-2):147-157.

The C5-binding polypeptides can be produced from cells by culturing a host cell transformed with the expression vector containing nucleic acid encoding the polypeptides, under conditions, and for an amount of time, sufficient to allow expression of the proteins. Such conditions for protein expression will vary with the choice of the expression vector and the host cell, and will be easily ascertained by one skilled in the art through routine experimentation. See, e.g., Current Protocols in Molecular Biology, Wiley & Sons, and Molecular Cloning—A Laboratory Manual—3rd Ed., Cold Spring Harbor Laboratory Press, New York (2001), which has comprehensive disclosure of recombinant DNA technology.

Following expression, the C5-binding polypeptide can be isolated or purified in a variety of ways known to those skilled in the art.

The C5-binding polypeptides, as well as other C5 inhibitors, used in a method of this disclosure specifically bind to a complement component C5 protein (e.g., human C5). The terms “specific binding” or “specifically binds” are known in the art and, briefly, can refer to two molecules forming a complex (e.g., a complex between a C5 inhibitor, including a C5-binding polypeptide, and a complement component C5 protein) that is relatively stable under physiologic conditions.

Methods for determining whether an antibody binds, including “specifically binds,” to an antigen and/or the affinity for an antibody to an antigen are known in the art. For example, the binding of an antibody to an antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Harlow and Lane (1988) “Antibodies: A Laboratory Manual” Cold Spring Harbor Laboratory Press, Cold Spring Harbor, N.Y.; Benny K. C. Lo (2004) “Antibody Engineering: Methods and Protocols,” Humana Press (ISBN: 1588290921); Borrebaek (1992) “Antibody Engineering, A Practical Guide,” W.H. Freeman and Co., NY; Borrebaek (1995) “Antibody Engineering,” 2nd Edition, Oxford University Press, NY, Oxford; Johne et al. (1993) J Immunol Meth 160:191-198; Jonsson et al. (1993) Ann Biol Clin 51:19-26; and Jonsson et al. (1991) Biotechniques 11:620-627.

Methods of making, identifying, purifying, modifying, etc. a C5 inhibitor are well known in the art.

The anti-C5 antibodies described herein and used for the methods and kits disclosed herein bind to complement component C5 (e.g., human C5) and inhibit the cleavage of C5 into fragments C5a and C5b.

In certain aspects, an anti-C5 antibody, or antigen binding fragment thereof, is provided. The antibody comprises CDR1, CDR2 and CDR3 domains of the heavy chain variable region having the sequence set forth in SEQ ID NO:15 or in SEQ ID NO:24, and CDR1, CDR2 and CDR3 domains of the light chain variable region having the sequence set forth in SEQ ID NO:16, for administration in an administration cycle comprising an induction phase followed by a maintenance phase, wherein:
the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or
the anti-C5 antibody, or antigen binding fragment thereof, is administered during the induction phase at a dose of 600 mg weekly for 1 week, starting at day 0, and is administered during the maintenance phase at a dose of 600 mg every week; or
the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

In some embodiments, an anti-C5 antibody comprises a heavy chain CDR1 comprising, or consisting of, the following amino acid sequence: GHIFSNYWIQ (SEQ ID NO:33). In some embodiments, an anti-C5 antibody described herein comprises a heavy chain CDR2 comprising, or consisting of, the following amino acid sequence: EILPGSGHTEYTENFKD (SEQ ID NO:29).

In some embodiments, an anti-C5 antibody described herein comprises a heavy chain variable region comprising the following amino acid sequence:

(SEQ ID NO: 24)
QVQLVQSGAEVKKPGASVKVSCKASGHIFSNYWIQWVRQAPGQGLEWMG
EILPGSGHTEYTENFKDRVTMTRDTSTSTVYMELSSLRSEDTAVYYCAR
YFFGSSPNWYFDVWGQGTLVTVSS.

In some embodiments, an anti-C5 antibody comprises a light chain variable region comprising the following amino acid sequence:

(SEQ ID NO: 16)
DIQMTQSPSSLSASVGDRVTITCGASENIYGALNWYQQKPGKAPKLLIY
GATNLADGVPSRFSGSGSGTDFTLTISSLQPEDFATYYCQNVLNTPLTF
GQGTKVEIK.

An anti-C5 antibody can, in some embodiments, comprise a variant human Fc constant region that binds to human neonatal Fc receptor (FcRn) with greater affinity than that of the native human Fc constant region from which the variant human Fc constant region was derived. For example, the Fc constant region can comprise one or more (e.g., two, three, four, five, six, seven, or eight or more) amino acid substitutions relative to the native human Fc constant region from which the variant human Fc constant region was derived. The substitutions can increase the binding affinity of an IgG antibody containing the variant Fc constant region to FcRn at pH 6.0, while maintaining the pH dependence of the interaction. See, e.g., Hinton et al. (2004) J Biol Chem 279:6213-6216 and Datta-Mannan et al. (2007) Drug Metab Dispos 35:1-9. Methods for testing whether one or more substitutions in the Fc constant region of an antibody increase the affinity of the Fc constant region for FcRn at pH 6.0 (while maintaining pH dependence of the interaction) are known in the art and exemplified in the working examples. See, e.g., Datta-Mannan et al. (2007) J Biol Chem 282(3):1709-1717; International Publication No. WO 98/23289; International Publication No. WO 97/34631; and U.S. Pat. No. 6,277,375.

Substitutions that enhance the binding affinity of an antibody Fc constant region for FcRn are known in the art and include, e.g., (1) the M252Y/S254T/T256E triple substitution described by Dall'Acqua et al. (2006) J Biol Chem 281: 23514-23524; (2) the M428L or T250Q/M428L substitutions described in Hinton et al. (2004) J Biol Chem 279:6213-6216 and Hinton et al. (2006) J Immunol 176:346-356; and (3) the N434A or T307/E380A/N434A substitutions described in Petkova et al. (2006) Int Immunol 18(12):1759-69. The additional substitution pairings: P257I/Q311I, P257I/N434H, and D376V/N434H are described in, e.g., Datta-Mannan et al. (2007) J Biol Chem 282(3):1709-1717.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three, or two) amino acid substitutions, insertions, or deletions relative to the native constant region from which it was derived. In some embodiments, the variant Fc constant region comprises one or more amino acid substitutions selected from the group consisting of: M252Y, S254T, T256E, N434S, M428L, V259I, T250I, and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434, or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan, or tyrosine for threonine at position 250; phenylalanine, tryptophan, or tyrosine for methionine at position 252; threonine for serine at position 254; glutamic acid for arginine at position 255; aspartic acid, glutamic acid, or glutamine for threonine at position 256; alanine, glycine, isoleucine, leucine, methionine, asparagine, serine, threonine, or valine for proline at position 257; histidine for glutamic acid at position 258; alanine for aspartic acid at position 265; phenylalanine for aspartic acid at position 270; alanine, or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan, or tyrosine for threonine at position 307; alanine, phenylalanine, isoleucine, leucine, methionine, proline, glutamine, or threonine for valine at position 308; alanine, aspartic acid, glutamic acid, proline, or arginine for leucine or valine at position 309; alanine, histidine, or isoleucine for glutamine at position 311; alanine or histidine for aspartic acid at position 312; lysine or arginine for leucine at position 314; alanine or histidine for asparagine at position 315; alanine for lysine at position 317; glycine for asparagine at position 325; valine for isoleucine at position 332; leucine for lysine at position 334; histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387; alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan, or tyrosine for methionine at position 428; lysine for histidine at position 433; alanine, phenylalanine, histidine, serine, tryptophan, or tyrosine for asparagine at position 434; and histidine for tyrosine or phenylalanine at position 436, all in EU numbering.

In one embodiment, the antibody binds to C5 at pH 7.4 and 25° C. (and, otherwise, under physiologic conditions) with an affinity dissociation constant (K_{D}) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95, or 0.975) nM.

In some embodiments, the K_{D} of the anti-C5 antibody, or antigen binding fragment thereof, is no greater than 1 (e.g., no greater than 0.9, 0.8, 0.7, 0.6, 0.5, 0.4, 0.3, or 0.2) nM.

In other embodiments, the [(K_{D} of the antibody for C5 at pH 6.0 at C)/(K_{D} of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500, or 8000).

In one embodiment, the anti-C5 antibody, or antigen binding fragment thereof, blocks the generation or activity of the C5a and/or C5b active fragments of a C5 protein (e.g., a human C5 protein). Through this blocking effect, the antibodies inhibit, e.g., the proinflammatory effects of C5a and the generation of the C5b-9 membrane attack complex (MAC) at the surface of a cell.

Methods for determining whether a particular antibody described herein inhibits C5 cleavage are known in the art. Inhibition of human complement component C5 can reduce the cell-lysing ability of complement in a subject's body fluids. Such reductions of the cell-lysing ability of complement present in the body fluid(s) can be measured by methods well known in the art such as, for example, by a conventional hemolytic assay such as the hemolysis assay described by Kabat and Mayer (eds.), “Experimental Immunochemistry, 2^{nd} Edition,” 135-240, Springfield, Ill., C C Thomas (1961), pages 135-139, or a conventional variation of that assay such as the chicken erythrocyte hemolysis method as described in, e.g., Hillmen et al. (2004) N Engl J Med 350(6):552. Methods for determining whether an antibody inhibits the cleavage of human C5 into forms C5a and C5b are known in the art and described in, e.g., Moongkarndi et al. (1982) Immunobiol 162:397; Moongkarndi et al. (1983) Immunobiol 165:323; Isenman et al. (1980) J Immunol 124(1):326-31; Thomas et al. (1996) Mol Immunol 33(17-18):1389-401; and Evans et al. (1995) Mol Immunol 32(16):1183-95. For example, the concentration and/or physiologic activity of C5a and C5b in a body fluid can be measured by methods well known in the art. Methods for measuring C5a concentration or activity include, e.g., chemotaxis assays, RIAs, or ELISAs (see, e.g., Ward and Zvaifler (1971) J Clin Invest 50(3):606-16 and Wurzner et al. (1991) Complement Inflamm 8:328-340). For C5b, hemolytic assays or assays for soluble C5b-9 as discussed herein can be used. Other assays known in the art can also be used. Using assays of these or other suitable types, candidate agents capable of inhibiting human complement component C5 can be screened.

An anti-C5 antibody can have a serum half-life in humans that is at least 20 (e.g., at least 21, 22, 23, 24, 25, 26, 27, 28, 29, 30, 31, 32, 33, 34, 35, or 36) days. Methods for measuring the serum half-life of an antibody are known in the art. See, e.g., Dall'Acqua et al. (2006) J Biol Chem 281: 23514-23524; Hinton et al. (2004) J Biol Chem 279:6213-6216; Hinton et al. (2006) J Immunol 176:346-356; and Petkova et al. (2006) Int Immunol 18(12):1759-69.

In some embodiments, an anti-C5 antibody, or antigen binding fragment thereof, has a serum half-life that is at least (e.g., at least 30, 35, 40, 45, 50, 55, 60, 65, 70, 75, 80, 85, 90, 95, 100, 125, 150, 175, 200, 250, 300, 400, 500) % greater than the serum half-life of eculizumab, e.g., as measured in one of the mouse model systems described in the working examples (e.g., the C5-deficient/NOD/scid mouse or hFcRn transgenic mouse model system).

In one embodiment, the antibody competes for binding with, and/or binds to the same epitope on C5 as a known antibody, such as eculizumab or an eculizumab variant. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same V_{H} and V_{L} or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope or to adjacent epitopes (e.g., as evidenced by steric hindrance).

Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

Pharmaceutical Compositions and Formulations

Compositions containing a C5 inhibitor, such as a C5-binding polypeptide, can be formulated as a pharmaceutical composition for administering to a subject. Any suitable pharmaceutical compositions and formulations, as well as suitable methods for formulating and suitable routes and suitable sites of administration, are within the scope of this invention, and are known in the art. Also, unless otherwise stated, any suitable dosage(s) and frequency of administration are contemplated.

The pharmaceutical compositions can include a pharmaceutically acceptable carrier (i.e., an excipient). A “pharmaceutically acceptable carrier” refers to, and includes, any and all solvents, dispersion media, coatings, antibacterial and antifungal agents, isotonic and absorption delaying agents, diluent, glidant, etc. The compositions can include a pharmaceutically acceptable salt, e.g., an acid addition salt or a base addition salt (see e.g., Berge et al. (1977) J Pharm Sci 66:1-19). The composition can be coated when appropriate.

In certain embodiments, the protein compositions can be stabilized and formulated as a solution, microemulsion, dispersion, liposome, lyophilized (freeze-dried) powder, or other ordered structure suitable for stable storage at high concentration. Sterile injectable solutions can be prepared by incorporating a C5-binding polypeptide, for use in the methods of this invention, in the required amount in an appropriate solvent with one or a combination of ingredients enumerated above, as required, followed by filtered sterilization. Generally, dispersions are prepared by incorporating a C5-binding polypeptide into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a C5 inhibitor polypeptide plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin. Non-protein C5 inhibitors can be formulated in the same, or similar, way.

The C5 inhibitor, including a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated at any desired concentration, including relatively high concentrations in aqueous pharmaceutical solutions. For example, a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated in solution at a concentration of between about 10 mg/mL to about 100 mg/mL (e.g., between about 9 mg/mL and about 90 mg/mL; between about 9 mg/mL and about 50 mg/mL; between about 10 mg/mL and about 50 mg/mL; between about 15 mg/mL and about 50 mg/mL; between about 15 mg/mL and about 110 mg/mL; between about 15 mg/mL and about 100 mg/mL; between about 20 mg/mL and about 100 mg/mL; between about 20 mg/mL and about 80 mg/mL; between about 25 mg/mL and about 100 mg/mL; between about 25 mg/mL and about 85 mg/mL; between about 20 mg/mL and about 50 mg/mL; between about 25 mg/mL and about 50 mg/mL; between about 30 mg/mL and about 100 mg/mL; between about 30 mg/mL and about 50 mg/mL; between about 40 mg/mL and about 100 mg/mL; between about 50 mg/mL and about 100 mg/mL; or between about 20 mg/mL and about 50 mg/mL); or at any suitable concentration. A C5-binding polypeptide used in the methods of this invention can be present in the solution at greater than (or at least equal to) about 5 (e.g., greater than, or at least equal to, about any of the following: 5, 6, 7, 8, 9, 10, 11, 12, 13, 14, 15, 16, 17, 18, 19, 20, 21, 22, 23, 24, 25, about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, or even 150) mg/mL. A C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated at a concentration of greater than about 2 (e.g., greater than about any of the following: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 or more) mg/mL, but less than about 55 (e.g., less than about any of the following: 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or less than about 5) mg/mL. Thus, in some embodiments, a C5-binding polypeptide used in the methods of this invention, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated in an aqueous solution at a concentration of greater than about 5 mg/mL and less than about 55 mg/mL. A C5-binding polypeptide used in the methods of this invention, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be formulated in an aqueous solution at a concentration of about 50 mg/mL. Any suitable concentration is contemplated. Methods for formulating a protein in an aqueous solution are known in the art and are described in, e.g., U.S. Pat. No. 7,390,786; McNally and Hastedt (2007), “Protein Formulation and Delivery,” Second Edition, Drugs and the Pharmaceutical Sciences, Volume 175, CRC Press; and Banga (1995), “Therapeutic peptides and proteins: formulation, processing, and delivery systems,” CRC Press.

Unless otherwise noted, the dosage level for a C5 inhibitor can be any suitable level. In certain embodiments, the dosage levels of an C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, for human subjects can generally be between about 1 mg per kg and about 100 mg per kg per patient per treatment, and can be between about 5 mg per kg and about 50 mg per kg per patient per treatment.

The plasma concentration in a patient, whether the highest level achieved or a level that is maintained, of a C5 inhibitor can be any desirable or suitable concentration. Such plasma concentration can be measured by methods known in the art. In certain embodiments, the concentration in the plasma of a patient (such as a human patient) of eculizumab or an eculizumab variant is in the range from about 25 μg/mL to about 500 μg/mL (such as between, for example, about 35 μg/mL to about 100 μg/mL). Such a plasma concentration of an anti-C5 antibody, in a patient can be the highest attained after administering the anti-C5 antibody, or can be a concentration of an anti-C5 antibody in a patient that is maintained throughout the therapy. However, greater amounts (concentrations) may be required for extreme cases and smaller amounts may be sufficient for milder cases; and the amount can vary at different times during therapy. In certain embodiments, the plasma concentration of eculizumab or an eculizumab variant can be maintained at or above about 35 μg/mL during treatment. In some embodiments, the plasma concentration of the plasma concentration of eculizumab or an eculizumab variant can be maintained at or above about 50 μg/mL during treatment.

In some embodiments, the plasma concentration of a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be maintained at or above about 200 nM, or at or above between about 280 nM to 285 nM, during treatment.

In other treatment scenarios, the plasma concentration of eculizumab or an eculizumab variant can be maintained at or above about 75 μg/mL during treatment. In the most serious treatment scenarios, the plasma concentration of eculizumab or an eculizumab variant can be maintained can be maintained at or above about 100 μg/mL during treatment.

In certain embodiments, the plasma concentration of a C5-binding polypeptide, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, can be maintained at or above about 200 nM to about 430 nM, or at or above about 570 nM to about 580 nM, during treatment.

In certain embodiments, the pharmaceutical composition is in a single unit dosage form. In certain embodiments, the single unit dosage form is between about 300 mg to about 1200 mg unit dosage form (such as about 300 mg, about 900 mg, and about 1200 mg) of a C5 inhibitor, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant. In certain embodiments, the pharmaceutical composition is lyophilized. In certain embodiments, the pharmaceutical composition is a sterile solution. In certain embodiments, the pharmaceutical composition is a preservative free formulation. In certain embodiments, the pharmaceutical composition comprises a 300 mg single-use formulation of 30 ml of a 10 mg/ml sterile, preservative free solution.

In certain embodiments, an anti-C5 full-length antibody (such as eculizumab or a variant thereof) is administered according to the following protocol: 600 mg via 25 to 45 minute IV infusion every 7+/−2 days for the first 4 weeks, followed by 900 mg for the fifth dose 7±2 days later, then 900 mg every 14±2 days thereafter. An anti-C5 antibody or polypeptide can be administered via IV infusion over 25 to 45 minute. In another embodiment, an anti-C5 polypeptide full-length antibody is administered according to the following protocol: 900 mg via 25 to 45 minute IV infusion every 7+/−2 days for the first 4 weeks, followed by 1200 mg for the fifth dose 7±2 days later, then 1200 mg every 14±2 days thereafter. An anti-C5 antibody can be administered via IV infusion over 25 to 45 minute. An exemplary pediatric dosing of, for example, an anti-C5 full-length antibody (such as eculizumab or a variant thereof), tied to body weight, is shown in Table 1:

TABLE 1
Exemplary dosing Recommendations in Pediatric
Patients for Full-length Antibodies

| Patient Body Weight | Induction | Maintenance |
|---|---|---|
| 40 kg and over | 900 mg weekly × 4 | 1200 mg at week 5; |
| | doses | then 1200 mg every 2 |
| | | weeks |
| 30 kg to less than | 600 mg weekly × 2 | 900 mg at week 3; |
| 40 kg | doses | then 900 mg every 2 |
| | | weeks |
| 20 kg to less than | 600 mg weekly × 2 | 600 mg at week 3; |
| 30 kg | doses | then 600 mg every 2 |
| | | weeks |
| 10 kg to less than | 600 mg weekly × 1 | 300 mg at week 2; |
| 20 kg | dose | then 300 mg every 2 |
| | | weeks |
| 20 kg to less than | 600 mg weekly × 1 | 600 mg at week 2; |
| 30 kg | dose | then 600 mg every 3 |
| | | weeks |

Note that in certain other embodiments the anti-C5 polypeptides that are not full-length antibodies and are smaller than a full-length antibodies can be administered at a dosage that correspond to the same molarity as the dosage for a full-length antibody.

The aqueous solution can have a neutral pH, e.g., a pH between, e.g., about 6.5 and about 8 (e.g., between and inclusive of 7 and 8). The aqueous solution can have a pH of about any of the following: 6.6, 6.7, 6.8, 6.9, 7, 7.1, 7.2, 7.3, 7.4, 7.5, 7.6, 7.7, 7.8, 7.9, or 8.0. In some embodiments, the aqueous solution has a pH of greater than (or equal to) about 6 (e.g., greater than or equal to about any of the following: 6.1, 6.2, 6.3, 6.4, 6.5, 6.6, 6.7, 6.8, 6.9, 7, 7.1, 7.2, 7.3, 7.4, 7.5, 7.6, 7.7, 7.8, or 7.9), but less than about pH 8.

In some embodiments, the C5 inhibitor, including a polypeptide inhibitor, is administered intravenously to the subject (the term “subject” is used herein interchangeably with the term “patient”), including by intravenous injection or by intravenous infusion. In some embodiments, the anti-C5 antibody is administered intravenously to the subject, including by intravenous infusion. In some embodiments, the C5 inhibitor, including a polypeptide inhibitor, is administered to the lungs of the subject. In some embodiments, the C5 inhibitor, including a polypeptide inhibitor, is administered to the subject by subcutaneous injection. In some embodiments, the inhibitor, including a polypeptide inhibitor, is administered to the subject by way of intraarticular injection. In some embodiments, the C5 inhibitor, including a polypeptide inhibitor, is administered to the subject by way of intravitreal or intraocular injection. In some embodiments, the inhibitor, including a polypeptide inhibitor, is administered to the subject by pulmonary delivery, such as by intrapulmonary injection (especially for pulmonary sepsis). Additional suitable routes of administration are also contemplated.

A C5 inhibitor, such as a C5-binding polypeptide, can be administered to a subject as a monotherapy. In some embodiments, the methods described herein can include administering to the subject one or more additional treatments, such as one or more additional therapeutic agents.

The additional treatment can be any additional treatment, including experimental treatments, or a treatment for a symptom of an infectious disease, such as fever, etc. The other treatment can be any treatment, any therapeutic agent, that improves or stabilizes the patient's health. The additional therapeutic agent(s) includes IV fluids, such as water and/or saline, acetaminophen, heparin, one or more clotting factors, antibiotics, etc. The one or more additional therapeutic agents can be administered together with the C5 inhibitor as separate therapeutic compositions or one therapeutic composition can be formulated to include both: (i) one or more C5 inhibitors such as C5-binding polypeptides and (ii) one or more additional therapeutic agents. An additional therapeutic agent can be administered prior to, concurrently, or after administration of the C5-binding polypeptide. An additional agent and a C5 inhibitor, such as C5-binding polypeptide, can be administered using the same delivery method or route or using a different delivery method or route. The additional therapeutic agent can be another complement inhibitor, including another C5 inhibitor.

In some embodiments, an inhibitor, such as a C5-binding polypeptide, can be formulated with one or more additional active agents useful for treating a complement mediated disorder caused by an infectious agent in a patient.

When a C5 inhibitor is to be used in combination with a second active agent, the agents can be formulated separately or together. For example, the respective pharmaceutical compositions can be mixed, e.g., just prior to administration, and administered together or can be administered separately, e.g., at the same or different times, by the same route or different route.

In some embodiments, a composition can be formulated to include a sub-therapeutic amount of a C5 inhibitor and a sub-therapeutic amount of one or more additional active agents such that the components in total are therapeutically effective for treating a complement mediated disorder caused by an infectious agent. Methods for determining a therapeutically effective dose of an agent such as a therapeutic antibody are known in the art.

The compositions can be administered to a subject, e.g., a human subject, using a variety of methods that depend, in part, on the route of administration. The route can be, e.g., intravenous (“IV”) injection or infusion, subcutaneous (“SC”) injection, intraperitoneal (“IP”) injection, pulmonary delivery such as by intrapulmonary injection (especially for pulmonary sepsis), intraocular injection, intraarticular injection, intramuscular (“IM”) injection, or any other suitable route.

A suitable dose of a C5 inhibitor, including a C5-binding polypeptide, which dose is capable of treating or preventing a complement mediated disorder caused by an infectious agent in a subject, can depend on a variety of factors including, e.g., the age, gender, and weight of a subject to be treated and the particular inhibitor compound used. Other factors affecting the dose administered to the subject include, e.g., the type or severity of the complement mediated disorder caused by an infectious agent. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will depend upon the judgment of the treating medical practitioner (e.g., doctor or nurse).

A C5 inhibitor can be administered as a fixed dose, or in a milligram per kilogram (mg/kg) dose. In some embodiments, the dose can also be chosen to reduce or avoid production of antibodies or other host immune responses against one or more of the active antibodies in the composition.

A pharmaceutical composition can include a therapeutically effective amount of a C5 inhibitor. Such effective amounts can be readily determined by one of ordinary skill in the art.

In certain embodiments, the dosing of a C5 inhibitor, such as eculizumab or a variant thereof, can be as follows: (1) administering to a patient with a complement mediated disorder caused by an infectious agent about 900 milligrams (mg) of eculizumab each week for the first 3 weeks, or (2) 1200 milligrams (mg) of eculizumab each week for the first 3 weeks and (3) followed by an about 1200 mg dose on weeks 4, 6, and 8. After an initial 8-week eculizumab treatment period, the treating medical practitioner (such as a physician) can optionally request (and administer) treatment with eculizumab about 1200 mg every other week for an additional 8 weeks. The patient can then be observed for 28 weeks following eculizumab treatment.

While in no way intended to be limiting, exemplary methods of administration for a single chain antibody such as a single chain anti-C5 antibody (that inhibits cleavage of C5) are described in, e.g., Granger et al. (2003) Circulation 108:1184; Haverich et al. (2006) Ann Thorac Surg 82:486-492; and Testa et al. (2008) J Thorac Cardiovasc Surg 136(4):884-893.

The terms “therapeutically effective amount” or “therapeutically effective dose,” or similar terms used herein are intended to mean an amount of a C5 inhibitor, such as eculizumab, an antigen-binding fragment thereof, an antigen-binding variant thereof, a polypeptide comprising the antigen-binding fragment of eculizumab or the antigen-binding fragment of an eculizumab variant, a fusion protein comprising the antigen binding fragment of eculizumab or the antigen-binding fragment of an eculizumab variant, or a single chain antibody version of eculizumab or of an eculizumab variant, that will elicit the desired biological or medical response.

In certain embodiments, for a patient with sepsis, a therapeutically effective amount of a C5 inhibitor can include an amount (or various amounts in the case of multiple administration) that improves the patient's chance of survival (by, e.g., any amount of time, such as one day or more), reduces C5a levels, reduces serum LDH levels, results in the patient having little to no organ failure, reduces levels of one or more of lactic acid, serum glutamic oxaloacetic transaminase (“SGOT”), creatine kinase, and creatine, reduces C-reactive protein level, reduces procalcitonin level, reduces serum amyloid A level, reduces mannan and/or antimannan antibody levels, reduces interferon-γ-inducible protein 10 (“IP-10”) level, increases levels of one or more of platelets and plasma bicarbonate level, decreases levels of one or more of the proinflammatory cytokines that are over-produced, or reduces other symptoms of the disease, or any combination thereof. All of these parameters can be ascertained or measured by known methods to a person skilled in the art.

In some embodiments, a composition described herein contains a therapeutically effective amount of a C5 inhibitor, such as a C5-binding polypeptide. In some embodiments, the composition contains any C5 inhibitor, such as a C5-binding polypeptide, and one or more (e.g., one, two, three, four, five, six, seven, eight, nine, ten, or eleven or more) additional therapeutic agents to treat or prevent a complement mediated disorder caused by an infectious agent, such that the composition as a whole is therapeutically effective. For example, a composition can contain a C5-binding polypeptide described herein and an immunosuppressive agent, wherein the polypeptide and agent are each at a concentration that when combined are therapeutically effective for treating or preventing a complement mediated disorder caused by an infectious agent in a subject.

### EXAMPLES

For this invention to be better understood, the following examples are set forth. These examples are for purposes of illustration only and are not be construed as limiting the scope of the invention in any manner.

### Example 1

### Eculizumab Treatment 1

From 1 mg per kg to 100 mg per kg per patient per treatment of a formulation comprising eculizumab (Alexion Pharmaceuticals, Inc., Cheshire Conn.) are administered to human patients diagnosed with sepsis by intravenous infusion; the C5a level in these patients are determined to be elevated. All of these patients are administered eculizumab for the first time early on in the disease state. At various days after, the disease level is determined by any methods known in the art.

Some of the parameters that can indicate improvement of the disease state include: improved patient's chance of survival, reduced C5a levels, reduced serum LDH levels, little to no organ failure, reduced levels of one or more of lactic acid, SGOT, creatine kinase, creatine, reduced C-reactive protein level, reduced procalcitonin level, reduced serum amyloid A level, reduced mannan and/or antimannan antibody levels, reduced interferon-γ-inducible protein 10 (“IP-10”) level, increased levels of one or more of platelets and plasma bicarbonate level, decreased levels of one or more of the proinflammatory cytokines that are over-produced, or reduced other symptoms of the disease, or any combination thereof. These parameters can be ascertained or measured by any methods known in the art.

The life expectancy of the patients receiving the formulation comprising eculizumab is increased by at least one day.

### Example 2

### Eculizumab Treatment 2

From 1 mg per kg to 100 mg per kg per patient per treatment of a formulation comprising eculizumab (Alexion Pharmaceuticals, Inc., Cheshire Conn.) are administered to human patients diagnosed with sepsis by intravenous infusion; the serum LDH level in these patients are determined to be elevated. All of these patients are administered eculizumab for the first time early on in the disease state. At various days after, the disease level is determined by any methods known in the art.

Some of the parameters that can indicate improvement of the disease state include: improved patient's chance of survival, reduced C5a levels, reduced serum LDH levels, little to no organ failure, reduced levels of one or more of lactic acid, SGOT, creatine kinase, creatine, reduced C-reactive protein level, reduced procalcitonin level, reduced serum amyloid A level, reduced mannan and/or antimannan antibody levels, reduced interferon-γ-inducible protein 10 (“IP-10”) level, increased levels of one or more of platelets and plasma bicarbonate level, decreased levels of one or more of the proinflammatory cytokines that are over-produced, or reduced other symptoms of the disease, or any combination thereof. These parameters can be ascertained or measured by any methods known in the art.

The life expectancy of the patients receiving the formulation comprising eculizumab is increased by at least one day.

### Example 3

### Clinical Trial 1

A clinical trial enrolls 100 patients with sepsis; the C5a level in these patients are determined to be elevated. Patients in the study receive 1200 milligrams (mg) of eculizumab (Alexion Pharmaceuticals, Inc., Cheshire Conn.) on day 1 of the study, followed by 1200 mg each week for the next 2 weeks, followed by a 1200 mg dose on weeks 4, 6, and 8. After an initial 8-week eculizumab treatment period, study investigators can optionally request treatment with eculizumab 1200 mg every other week for an additional 8 weeks. The administration to patients of the eculizumab is performed by intravenous infusion. The patients are observed and tested for C5a levels every 6 hours after the first administration, until 72 hours after that first administration.

Some of the parameters that can indicate improvement of the disease state include: improved patient's chance of survival, reduced C5a levels, reduced serum LDH levels, little to no organ failure, reduced levels of one or more of lactic acid, SGOT, creatine kinase, creatine, reduced C-reactive protein level, reduced procalcitonin level, reduced serum amyloid A level, reduced mannan and/or antimannan antibody levels, reduced interferon-γ-inducible protein 10 (“IP-10”) level, increased levels of one or more of platelets and plasma bicarbonate level, decreased levels of one or more of the proinflammatory cytokines that are over-produced, or reduced other symptoms of the disease, or any combination thereof. These parameters can be ascertained or measured by any methods known in the art.

The life expectancy of the patients receiving eculizumab is increased by at least one day.

### Example 4

### Clinical Trial 2

A clinical trial enrolls about 100 patients with sepsis; the serum LDH level in these patients are determined to be elevated. Patients in the study receive 1200 milligrams (mg) of eculizumab (Alexion Pharmaceuticals, Inc., Cheshire Conn.) on day 1 of the study, followed by 1200 mg each week for the next 2 weeks, followed by a 1200 mg dose on weeks 4, 6, and 8. After an initial 8-week eculizumab treatment period, study investigators can optionally request treatment with eculizumab 1200 mg every other week for an additional 8 weeks. The administration to patients of the eculizumab is performed by intravenous infusion. The patients are observed and tested for serum LDH levels every 6 hours after the first administration, until 72 hours after that first administration.

Some of the parameters that can indicate improvement of the disease state include: improved patient's chance of survival, reduced C5a levels, reduced serum LDH levels, little to no organ failure, reduced levels of one or more of lactic acid, SGOT, creatine kinase, creatine, reduced C-reactive protein level, reduced procalcitonin level, reduced serum amyloid A level, reduced mannan and/or antimannan antibody levels, reduced interferon-γ-inducible protein 10 (“IP-10”) level, increased levels of one or more of platelets and plasma bicarbonate level, decreased levels of one or more of the proinflammatory cytokines that are over-produced, or reduced other symptoms of the disease, or any combination thereof. These parameters can be ascertained or measured by any methods known in the art.

The life expectancy of the patients receiving eculizumab is increased by at least one day.

### Example 5

### Eculizumab Treatment 1 (VHF)

From 1 mg per kg and to 100 mg per kg per patient per treatment of a formulation comprising eculizumab (Alexion Pharmaceuticals, Inc., Cheshire Conn.) are administered to human patients diagnosed with Ebola hemorrhagic fever (also known as Ebola virus disease) intravenously. Placebo is administered intravenously to a control group of human patients with Ebola hemorrhagic fever. All of these patients are administered eculizumab for the first time early on in the disease state. At various days after, the disease level is determined by any methods known in the art.

Some of the parameters that can indicate improvement of the disease state include: improved patient's chance of survival, decreased hemolysis, decreased disseminated intravascular coagulation, reduced complement levels, decreased levels of the cytokines that are over-produced, decreased thrombolitic microangiopathy, improved renal functions, or reduced other symptoms of the disease, or any combination thereof. These parameters can be ascertained or measured by any methods known in the art.

The life expectancy of the patients receiving the formulation comprising eculizumab is increased by least one day.

### Example 6

### Eculizumab Treatment 2 (VHF)

From 5 mg per kg and to 50 mg per kg per patient per treatment of a formulation comprising eculizumab (Alexion Pharmaceuticals, Inc., Cheshire Conn.) are administered intravenously to human patients diagnosed with Ebola hemorrhagic fever (also known as Ebola virus disease) and suffering hemorrhage and hemolysis. Placebo is administered intravenously to a control group of human patients with Ebola hemorrhagic fever and suffering hemorrhage and hemolysis. The degree of hemolysis is determined prior to the first administration of eculizumab. At various days after, the disease level, including the level of hemolysis, is determined by any methods known in the art.

Some of the parameters that can indicate improvement of the disease state include: improved patient's chance of survival, decreased hemolysis, decreased disseminated intravascular coagulation, reduced complement levels, decreased levels of the cytokines that are over-produced, decreased thrombolitic microangiopathy, improved renal functions, or reduced other symptoms of the disease, or any combination thereof. These parameters can be ascertained or measured by any methods known in the art.

The life expectancy of the patients receiving the formulation comprising eculizumab is increased by least one day.

### Example 7

### Eculizumab Treatment 3 (VHF)

Patients receive a three unit dosage forms of a 300 mg formulation comprising eculizumab (Alexion Pharmaceuticals, Inc., Cheshire Conn.) or 900 mg intravenously on day 0. These human patients area diagnosed with Ebola hemorrhagic fever (also known as Ebola virus disease). Placebo is administered intravenously to control group of human patients with Ebola hemorrhagic fever infection. All of these patients are administered eculizumab for the first time early on in the disease state. The levels of complement are determined in each patient by any method known in the art prior to the administration of eculizumab. Only those patients with elevated complement levels are used in this study. At various days after, the disease level is determined and the complement level is determined, by any methods known in the art. Those administered with eculizumab tend to have better outcome and tend to have decreased complement levels. Once the complement levels have reached normal levels, no further eculizumab is administered to those patients.

Some of the parameters that can indicate improvement of the disease state include: improved patient's chance of survival, decreased hemolysis, decreased disseminated intravascular coagulation, reduced complement levels, decreased levels of the cytokines that are over-produced, decreased thrombolitic microangiopathy, improved renal functions, or reduced other symptoms of the disease, or any combination thereof. These parameters can be ascertained or measured by any methods known in the art.

The life expectancy of the patients receiving the formulation comprising eculizumab is increased by least one day.

### Example 8

### Clinical Trial (VHF)

A 28-week, open-label, multi-center trial enrolls about 200 patients with Ebola hemorrhagic fever infection. Patients in the study receive 900 milligrams (mg) of eculizumab (Alexion Pharmaceuticals, Inc., Cheshire Conn.) each week for the first 3 weeks, followed by a 1200 mg dose on weeks 4, 6, and 8. After an initial 8-week eculizumab treatment period, study investigators can optionally request treatment with eculizumab 1200 mg every other week for an additional 8 weeks. All patients in the study are observed for 28 weeks following eculizumab treatment. The administration to patients of the eculizumab is performed intravenously.

Some of the parameters that can indicate improvement of the disease state include: improved patient's chance of survival, decreased hemolysis, decreased disseminated intravascular coagulation, reduced complement levels, decreased levels of the cytokines that are over-produced, decreased thrombolitic microangiopathy, improved renal functions, or reduced other symptoms of the disease, or any combination thereof. These parameters can be ascertained or measured by any methods known in the art.

The life expectancy of the patients receiving eculizumab is increased by least one day.

### Example 9

### A Phase 2 Open-Label, Multicenter Clinical Trial in STEC-HUS Patients

This is an open-label, non-comparative, multicenter phase 2 clinical trial designed to assess the safety and efficacy of eculizumab in patients with STEC-HUS. Eligibility criteria were established in accordance with the urgent need to provide eculizumab in a very sick patient population. They included a diagnosis of Shiga toxin in the context of EHEC infection, evidence of thrombocytopenia and hemolysis and involvement of at least one target organ including kidney and/or brain and/or thrombosis. There was no specific requirement for severity of disease or prior therapy with Plasma Exchange or Plasma Infusion (PE/PI). However physicians requested access to commercially available eculizumab based on patients' poor response to PE/PI and other interventions or clinical evidence that the severity of disease would make symptomatic treatment with PE/PI unlikely to be effective.

Study procedures were prospectively defined. The protocol provided a treatment duration of 8 weeks. The protocol provided the option of an additional 8 weeks of treatment for patients who, in the opinion of the investigator, may have benefited from a longer treatment because of residual abnormalities in kidney or central nervous system (CNS) function.

All data were collected from the day of the first symptoms of STEC-HUS until the end of the 28-week study period. Analyses were to be conducted on all patients and stratified by several factors. Patients were to be evaluated for protocol-specified neurologic involvement and Baseline and outcome criteria by a trained and certified neurology specialist.

There were 3 periods in the study: Screening, Treatment Period (with optional extended dosing) and the Post-Treatment Period. All patients were followed for 28 weeks.

Objectives

The primary objective of the study is to assess the short-term (8 weeks) efficacy and safety of eculizumab in STEC-HUS patients.

Secondary objectives include assessing the safety and efficacy profile of eculizumab on short-term and long-term outcomes of STEC-HUS, and assessing the prognostic value of clinical manifestations of STEC-HUS on short-term and long-term outcomes of STEC-HUS.

Study Design

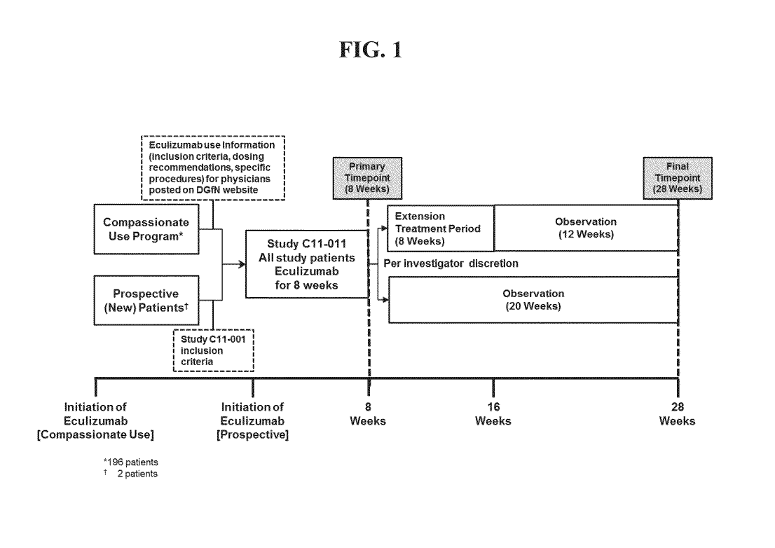

The overall study design, treatments and study duration is as follows. See also FIG. 1 to FIG. 3. Duration of treatment: Eculizumab treatment commenced with the first eculizumab dose and continued for at least eight weeks. After this initial treatment period, patients demonstrating residual abnormality of kidney or central nervous system (CNS) function were allowed to continue eculizumab treatment for an additional eight weeks if the investigator felt that a longer treatment period would benefit the patient.

TABLE 2
Eculizumab Dose and Mode of Administration

| Group | Induction Dose | Maintenance Dose |
|---|---|---|
| Adults | 900 mg weekly for 4 weeks | 1200 mg at Week 5 |
| | | then |
| | | every 2 weeks |

Adolescent and Pediatric Patients ≧2 months old

| | | | |
|---|---|---|---|
| ≧40 | kg | 900 mg weekly for 4 weeks | 1200 mg at Week 5 |
| | | | then |
| | | | every 2 weeks |
| 30-<40 | kg | 600 mg weekly for 2 weeks | 900 mg at Week 3 |
| | | | then |
| | | | every 2 weeks |
| 20-<30 | kg | 600 mg weekly for 2 weeks | 600 mg at Week 3 |
| | | | then |
| | | | every 2 weeks |
| 10-<20 | kg | 600 mg weekly for 1 week | 300 mg at Week 2 |
| | | | then |
| | | | every 2 weeks |
| 5-<10 | kg | 300 mg weekly for 1 week | 300 mg at Week 2 |
| | | | then |
| | | | every 3 weeks |

Dose (mg) by Study Day and Body Weight

| | | | | | Day | | Day | Day | Day | |
|---|---|---|---|---|---|---|---|---|---|---|
| | | Day 0 | Day 7 | Day 14 | 21 | Day 28 | 35 | 42 | 49 | Day 56¹ |
| Adults | | 900 | 90 | 90 | 90 | 120 | | 12 | | 1200 |
| | | | Adolescent and Pediatric Patients >2 months old | | | | | | | |
| ≧40 | kg¹ | 900 | 900 | 900 | 900 | 1200 | | 1200 | | 1200 |
| 30-<40 | kg | 60 | 600 | 900 | | 900 | | 900 | | 900 |
| 20-<30 | kg | 60 | 600 | 600 | | 600 | | 600 | | 600 |
| 10-<20 | kg | 60 | 300 | | 300 | | 300 | | 300 | |
| 5-<10 | kg | 30 | 300 | | | 300 | | | 300 | |

¹Same dose continued for selected patient treated long-term (16 weeks).

In adults and older children (40 kg), the induction and maintenance dosing with eculizumab 900 and 1200 mg was administered via intravenous (IV) infusion over approximately 35 minutes.

In pediatric patients (40 kg), the induction dose(s) were administered via IV infusion, over approximately 1 to 4 hours depending on body weight and the PI's discretion. The maintenance doses selected also were administered via IV infusion, over approximately 1 to 4 hours depending on body weight and Investigator discretion.

196 patients were retrospectively enrolled after signing informed consent form. All received commercially available eculizumab prior to enrollment and at least 1 dose of eculizumab as investigational product following study entry. Two patients were enrolled prospectively, to a total of 198 patients. This represents the IIT/safety population.

At screening, the following are to be collected: medical history, demographics, historical data review, administration/confirmation of N. meningitidis vaccination and prophylactic antibiotics; neurology assessments, clinical laboratories, safety, seizure assessment, disease-specific information.

Fixed dosing of eculizumab based on body weight cohorts were administered. Adjustment of dose to accommodate patient growth was possible.

Eculizumab can be administered intravenously (IV) according to the regimens described below:

Cohort 1: If weight≧40 kg: Induction: 900 mg weekly×4; Maintenance: 1200 mg Wk5; then 1200 mg every 2 weeks.

Cohort 2: If weight 30≦40 kg: Induction: 600 mg weekly×2; Maintenance: 900 mg Wk3; then 900 mg every 2 weeks.

Cohort 3: If weight 20≦30 kg: Induction: 600 mg weekly×2; Maintenance: 600 mg Wk3; then 600 mg every 2 weeks.

Cohort 4: If weight 10≦20 kg: Induction: 600 mg weekly×1; Maintenance: 300 mg Wk2; then 300 mg every 2 weeks.

Cohort 5: If weight 5≦10 kg: Induction 300 mg weekly×1; Maintenance: 300 mg week 2; then 300 mg every 3 weeks.

Patients enrolled prospectively were not permitted to receive plasma exchange or plasma infusion (PE/PI) (fresh frozen plasma) within the first 96-hour period following the first eculizumab dose unless there was a compelling medical need as assessed by clinical evidence of worsening of any clinical parameters. If the treating physician believed that PE/PI were medically indicated the following (Table 3) eculizumab supplemental treatment was to be administered:

TABLE 3
Supplemental Dose of Eculizumab After PE/PI

| | | Supplemental | |
|---|---|---|---|
| | | Eculizumab Dose | Timing of |
| Type of | Most Recent | With Each PE/PI | Supplemental |
| Intervention | Eculizumab Dose | Intervention | Eculizumab Dose |
| Plasmapheresis | 300 mg | 300 mg per each | Within 60 |
| or plasma | | plasmapheresis | minutes after |
| exchange | | or plasma | each |
| | | exchange | plasmapheresis |
| | | session | or plasma |
| | 600 mg or more | 600 mg per each | exchange |
| | | plasmapheresis | |
| | | or plasma | |
| | | exchange | |
| | | session | |
| Fresh | 300 mg or more | 300 mg per | 60 minutes prior |
| Frozen | | each unit of | to each 1 unit of |
| Plasma | | fresh frozen | fresh frozen |
| infusion | | plasma | plasma infusion |

Eculizumab (h5G1.1-mAb) is a humanized IgG2/4 kappa antibody, consisting of two 448 amino acid heavy chains and two 214 amino acid light chains. The heavy chains are comprised of human IgG2 sequences in constant region 1, the hinge, and the adjacent portion of constant region 2, and human IgG4 sequences in the remaining part of constant region 2 and constant region 3. The light chains are comprised of human kappa sequences. The variable chains consist of human framework regions with grafted murine complementarity-determining regions, which form the antigen-binding site.

Eculizumab was prepared in vials, packaged in kits, and shipped from Almac Clinical Services in Durham, N.C., USA to Almac Clinical Pharma Services in Craigavon, UK. These supplies were then shipped to Arvato in Germany for distribution to the clinical sites. Each single 30 mL vial contained a solution concentration of 10 mg/mL (300 mg of active ingredient) and had enough solution to withdraw the indicated 30 mL.

All patients were to be vaccinated against meningococcal infection with a quadrivalent meningococcal conjugate vaccine (preferably Menveo®), unless previously vaccinated against meningococcal infection, and all patients who continued treatment with eculizumab beyond 8 weeks were to receive a booster vaccination with a quadrivalent meningococcal conjugate vaccine (preferably Menveo®) at Week 8. Moreover, all patients were to receive prophylactic antibiotic (azithromycin or age-appropriate antibiotics) until 14 days after initial vaccination.

According to the local recommendations in Germany, antibiotic prophylaxis against meningococcal infection was to be strongly recommended for patients who were less than 18 years of age during eculizumab therapy and for 4 weeks after the last eculizumab administration, unless such antibiotic treatment was contraindicated. Palliative and supportive care was permitted during the course of the study for underlying conditions. If the investigator believed that plasmapheresis (PPH) or plasma exchange (PE), and fresh frozen plasma (FFP) was medically indicated, specific instructions for eculizumab were provided.

The following concurrent medications were to be prohibited during the study: Intravenous immunoglobulin (IVIg; unless for an unrelated medical need, e.g., hypogammaglobulinemia); Rituximab; and Immunoadsorption. Patients were not permitted to receive PT within the first 96-hour period following the first eculizumab dose unless there was a compelling medical need.

Patients were infused with eculizumab under the supervision of a physician or designee, to ensure that the patient received the appropriate dose at the appropriate time points during the trial. Treating physicians became either the PI or a Subinvestigator at their sites once the study began.

All laboratory assessments related to safety and efficacy, ECG, vital sign, and neurological function measurements were performed using accepted and consistent methods within each investigational site.

Criteria for Evaluation: Primary Endpoints

The primary efficacy endpoint in the protocol was the improvement in systemic TMA and Vital Organ Involvement at 8 weeks of treatment (complete plus partial responders). The response rate was also to be assessed at Week 16 and Week 28.

Complete response at Week (wk or Wk; wks or Wks for Weeks) 8 [Week 16, Week 28] was defined as:
Hematologic normalization (platelet count≧150×10⁹/L at any 2 consecutive measures up to Week 8 [Week 16, Week 28])
Clinically important improvement up to Week 8 (Week 16, Week 28) in all of the affected major vital organs: brain, kidney, thrombosis when abnormal at Baseline and with baseline abnormality plausibly related to EHEC event, defined as follows:
   Clinically important improvement from Baseline in renal function was defined as: ≧25% decrease in serum creatinine at any 2 consecutive measures up to Week 8 (Week 16, Week 28), normalization of serum creatinine (within lab normal range) at any 2 consecutive measures up to Week 8 (Week 16, Week 28), or elimination of dialysis up to Week 8 (Week 16, Week 28)
   Clinically important improvement from Baseline in neurologic function established by trained and certified neurology specialist and defined as: MRS with shift from score of 5 or 4 to 3 or less, shift from 3 or 2 to 1 or less (note that a MRS of 0 or 1 is normal) in the first 8 weeks (16 weeks, 28 weeks) (note: all reported MRS data were used for analysis regardless of whether reported as being provided by a trained and verified neurology specialist); or, if seizures:
      For patients with seizures receiving therapeutic coma prior to eculizumab initiation (at Baseline): no seizures and no therapeutic coma, inclusive, during the Week 8 visit window [Week 16 visit window, Week 28 visit window]
      For patients who became seizure-free on unchanged oral AEDs for 3 days immediately prior to start of eculizumab: no seizures during the Week 8 visit window (Week 16 visit window, Week 28 visit window) with ≧30% reduction in AEDs in the first 8 weeks (16 weeks, 28 weeks) (An independent expert assessed AED use, in particular, the assessment of a 30% reduction in such use.)
      For patients with seizures and receiving AEDs prior to eculizumab initiation: no seizures during the Week 8 visit window (Week 16 visit window, Week 28 visit window)
   Thrombotic events: no thrombotic events after Day 14 to the end of Week 8 (Week 16, Week 28)
No clinically important worsening in brain, kidney, thrombosis, where clinically important worsening was defined as follows:
   Kidney—≧25% increase in serum creatinine at any 2 consecutive measures or new dialysis after Day 14 to the end of Week 8 (Week 16, Week 28)
   Thrombosis—any new thrombotic events after Day 14 to the end of Week 8 (Week 16, Week 28).
   Brain—An increase in the MRS, from 0 or 1 at Baseline to 2 or higher, or from 2 or 3 at Baseline to 4 or higher, after Day 14 to the end of Week 8 (Week 16, Week 28), or seizures defined as follows:
      Patient who had no seizures prior to treatment with eculizumab—any new onset seizure beginning after Day 14 to the end of Week 8 (Week 16, Week 28)
      For patients who became seizure-free on unchanged oral AEDs for 3 days immediately prior to start of eculizumab: need to add an additional AED or any new seizures during the Week 8 visit window (Week 16 visit window, Week 28 visit window)
      For patients with seizures and receiving AEDs prior to eculizumab initiation: need to add an additional AED or new therapeutic coma needed during the Week 8 visit window (Week 16 visit window, Week 28 visit window)
      For patients with seizures receiving therapeutic coma prior to eculizumab initiation: progressive (worsening) brain swelling on computed tomography/magnetic resonance imaging (CT/MRI) during the Week 8 visit window (Week 16 visit window, Week 28 visit window)

Partial response at Week 8 (Week 16, Week 28) was defined as follows:
Hematologic improvement (≧25% increase in platelet count at any 2 consecutive measures) or hematologic normalization at any 2 consecutive measures during the first 8 weeks (16 weeks, 28 weeks)
Clinically important improvement in none, 1, or more affected major organs: brain, kidney, and thrombosis when abnormal at Baseline and when Baseline abnormality plausibly related to the EHEC event during the first 8 weeks (16 weeks, 28 weeks)
No clinically important worsening in brain, kidney, thrombosis during the first 8 weeks (16 weeks, 28 weeks)

Secondary Endpoints:
Hematologic improvement (≧25% increase in platelet count at any 2 consecutive measures) or hematologic normalization at any 2 consecutive measures during the first 8 weeks (16 weeks, 28 weeks)
Clinically important improvement in none, 1, or more affected major organs: brain, kidney, and thrombosis when abnormal at Baseline and when Baseline abnormality plausibly related to the EHEC event during the first 8 weeks (16 weeks, 28 weeks)
No clinically important worsening in brain, kidney, thrombosis during the first 8 weeks (16 weeks, 28 weeks)
Global assessment of renal function
Global assessment of neurological function
TMA event-free status for ≧6 weeks
TMA intervention rate
New ventilator requirement
New dialysis after Day 14 of eculizumab treatment

Other Endpoints:

The following efficacy endpoints were also to be assessed at Weeks 8, 16, and 28:
Overall normalization, defined as: hematologic normalization in patients with platelets<150×10⁹/L at Baseline, creatinine normalization in patients with abnormal (high) creatinine at Baseline or on dialysis at Baseline, and a return to 0 or 1 in the MRS for patients with a baseline score of 2 or higher
Complete hematologic response, defined as normalization of platelet count and LDH sustained for at least 2 consecutive measurements obtained at least 4 weeks apart, assessed in all patients. This endpoint was to be assessed only at Week 28.
Serum creatinine (mg/dL), both as a continuous variable and as responders (defined 2 ways: (1) decrease from Baseline ≧25% at any 2 consecutive measures only in those patients not on dialysis at Baseline, and (2) return to normal range observed at any 2 consecutive measures in all patients)
eGFR, (mL/min/1.73 m²), both as a continuous variable and as responders (defined 3 ways: [1] increase from Baseline ≧15 mL/min/1.73 m² at any 2 consecutive measures only in those patients not on dialysis at Baseline, [2] increase from Baseline to ≧60 mL/min/1.73 m² at any 2 consecutive measures in all patients, and [3] increase from Baseline to ≧90 mL/min/1.73 m² at any 2 consecutive measures in all patients)
   The eGFR was calculated using the modification of diet in renal disease (MDRD) formula in patients 18 years or older as follows:

eGFR (mL/min/1.73 m²)=186×[serum creatinine (mg/dL)]^{-1.154}×[Age]^{-0.203}×[0.742 if patient is female]×□[1.212 if patient is African-American]
The eGFR was calculated using the Schwartz formula in patients less than 18 years of age as follows:

eGFR (mL/min/1.73 m²)=0.413×[height (cm)/serum creatinine (mg/dL)]
CKD stage, calculated from eGFR (mL/min/1.73 m²) and dialysis as follows:

Stage 1=eGFR≧90

Stage 2=60≦eGFR<90

Stage 3a=45≦eGFR<60

Stage 3b=30≦eGFR<45

Stage 4=15≦eGFR<30

Stage 5=eGFR<15 or dialysis
Acute kidney injury stage (note that the reported stage of 1, 2, or 3 was used for analysis)
Platelets, both as a continuous variable and as responders/hematologic improvement in patients with abnormal (low) platelets at Baseline (≧25% increase in platelet count at any 2 consecutive measures)
Lactate dehydrogenase, both as a continuous variable and as responders in patients with abnormal (high) LDH at Baseline (return to normal range at any 2 consecutive measures)
Hemoglobin (Hgb), both as a continuous variable and as responders in patients with abnormal (low) Hgb at Baseline (defined 2 ways: (1) increase from Baseline ≧20 g/L at any 2 consecutive measures, and (2) return to normal range observed at any 2 consecutive measures)
MRS, both as an ordinal variable and return to normal (0 or 1) in patients with a score of 2 or higher at Baseline
For patients receiving PE/PI at Baseline, the time to discontinuation
For patients receiving dialysis at Baseline, the time to discontinuation
For patients with seizures at Baseline, the time to stop having seizures
For patients in a therapeutic coma at Baseline, the time to discontinuation
The number and percent of patients with thrombotic events after Day 14 through Week 8, Week 16, and Week 28

Safety Endpoints: Safety was assessed by examination of the following safety parameters: Adverse events and Meningococcal events.

To find meningococcal events, the AE dataset was searched for the following MedDRA preferred terms (PTs): Meningitis meningococcal, Meningococcal bacteremia, Meningococcal infection, Meningococcal sepsis, Neisseria infection, Septic arthritis neisserial, Meningococcal carditis, Encephalitis meningococcal, Endocarditis meningococcal, Myocarditis meningococcal, Optic neuritis meningococcal, Pericarditis meningococcal, and Neisseria test positive. In addition, a medical review was done to ensure that no relevant events were missed.

The Investigator was responsible for reporting all AEs and serious adverse events (SAE) observed or reported during the study regardless of their relationship to eculizumab, their relationship to the patients' underlying STEC-HUS disease, or their clinical significance.

An AE was defined as any untoward medical occurrence in a patient enrolled into this study regardless of its causal relationship to study treatment. Patients were instructed to contact the PI or Sub-investigator at any time after enrollment if any symptoms developed.

A treatment-emergent AE (TEAE) was defined as any event not present prior to exposure to eculizumab or any event already present that worsened in either intensity or frequency following exposure to eculizumab.

An SAE was defined as any event that results in death, is immediately life-threatening, requires inpatient hospitalization or prolongation of existing hospitalization, results in persistent or significant disability/incapacity, or is a congenital anomaly/birth defect.

All AEs that occurred at or after the first receipt of study drug, either prior to or after enrollment in the study, were reported in detail in the patient's source/chart and on the appropriate CRF and followed to satisfactory resolution or until the PI or Sub-investigator deemed the event to be chronic or the patient to be stable. The description of the AE included the onset date, date of resolution, severity, treatment received for the AE, and the likelihood of relationship of the AE to eculizumab.

Severity of each AE was rated by the PI as mild, moderate, or severe using the following criteria. Mild: events require minimal or no treatment and do not interfere with the patient's daily activities. Moderate: events result in a low level of inconvenience or concerns with the therapeutic measures. Moderate events may cause some interference with functioning. Severe: events interrupt a patient's usual daily activity and may require systemic drug therapy or other treatment. Severe events are usually incapacitating.

The relationship or association of eculizumab in causing or contributing to the AE was characterized by the PI using the following classification and criteria. Unrelated: This relationship suggests that there is no association between eculizumab and the reported event. Unlikely: This relationship suggests that the clinical picture is highly consistent with a cause other than eculizumab but attribution cannot be made with absolute certainty and a relationship between the Investigational Product and AE cannot be excluded with complete confidence. Possible: This relationship suggests that treatment with eculizumab caused or contributed to the AE, i.e., the event follows a reasonable temporal sequence from the time of drug administration and/or follows a known response pattern to eculizumab, but could also have been produced by other factors. Probable: This relationship suggests that a reasonable temporal sequence of the event with eculizumab administration exists and the likely association of the event with the Investigational Product. This will be based upon the known pharmacological action of eculizumab, known or previously reported adverse reactions to eculizumab or its class of drugs, or judgment based on the Investigator's clinical experience. Definite: This relationship suggests that a definite causal relationship exists between eculizumab administration and the AE, and other conditions (concurrent illness, progression/expression of disease state, or concurrent medication reaction) do not appear to explain the event. Adverse events that were deemed by the PI to be possible, probable or definite shall be considered related to eculizumab.

Every effort was made to correlate an abnormal laboratory test result with a clinical diagnosis (e.g., elevated blood glucose with diabetes). The clinical diagnosis was reported (e.g., “Type II diabetes”) rather than the laboratory abnormality. In cases where a laboratory abnormality could not be linked to a disease-state or condition, the laboratory test result was reported as the AE (e.g., hyperglycemia).

Drug Concentration Measurements It can be observed that exposure in STEC-HUS patients is generally within the target concentration range (35-700 μg/mL).

Data Quality Assurance The investigators were to maintain adequate medical records, accurate source documentation, and CRFs for the patients treated as part of the research under this protocol. Clinical monitors were to visit study sites at periodic intervals, in addition to maintaining necessary telephone and written contact as described in the monitoring plan. During the visits, the monitors were to review study records and source documentation, and discuss the conduct of the study with the study site staff, including the Investigator. The Sponsor and Alcedis GmBH were to monitor all aspects of the study for compliance with applicable government regulations, ICH's GCP, and Alcedis GmBH standard operating procedures.

The CRF was a web-based electronic CRF. The site users were trained by the site monitors and online instructions were available. Each user was given a unique login account. The programming for the CRF allowed the investigator to indicate if a requested item was not available or was not applicable, but blank spaces were not permitted. Incomplete dates were also not allowed, so Alcedis GmBH instructed the sites to enter (1) If the Day was missing, the site was to use 1 and if both (2) Day and month were missing, the site was to use 01 July. All sites were instructed to enter comments to document such date imputation. These comments were used to create a flag variable for date imputation in the SAS datasets. Corrections to the CRF were tracked in an audit trail with the user's login name and date and time the entry or correction was made. Investigators electronically signed every CRF page. The study monitors reviewed the CRF during site visits. At the completion of the study, a copy of the CRF as an Adobe Acrobat (i.e., portable document format [PDF]) file on a computer disc will be placed in the investigators' site files at the study centers.

Statistical Methods Planned in the Protocol and Determination of Sample Size

Study Population Definitions

Three populations were defined and used in various analyses in this study: Safety set, Full-Analysis set (or intent-to-treat [ITT]), and Per-Protocol (PP) set.

The Full Analysis set or ITT population was identical to the safety population and consisted of all patients enrolled who signed an informed consent and received at least 1 dose of eculizumab. The ITT population (same as full analysis set) was used for the analysis of efficacy data and was considered the primary analysis population.

The PP population consisted of all ITT patients who satisfied the following criteria: Received at least 8 weeks of dosing with eculizumab, defined as 7 or more doses of eculizumab in the first 8 weeks for adult patients (for pediatric patients, the planned number of doses was given by the recommended treatment schedule and the definition was modified accordingly). Exceptions were that patients who received less than 8 weeks of dosing with eculizumab either due to death or discontinuation due to an AE considered related to eculizumab were included in the PP population and counted as failures for the primary endpoint. Discontinuation due to death was determined by the 8-, 16-, and 28-week study disposition CRF's. Patients who discontinued due to an AE were determined by the 8-, 16-, and 28-week study disposition CRF's, and their AE's were reviewed by a physician to determine if there were any AE's possibly, probably, or definitely related to eculizumab that could have been the reason for discontinuation. Received 80%-125% of the planned total dose for the first 8 weeks. The planned dose was determined by the recommended treatment schedule. For patients 18 years and older, and patients<18 years old who weighed≧40 kg, the planned dose for the first 8 weeks was 7,200 mg. Did not take any prohibited concurrent medications during the 28-week study period (IVIg [unless for an unrelated medical need], rituximab, or immunoabsorption). Met all inclusion/exclusion criteria.

The safety population consisted of all patients enrolled who signed an informed consent and received at least 1 dose of eculizumab. The safety population was used for the analysis of safety data.

Other Efficacy Analyses For each of the endpoints of serum creatinine, eGFR, platelets, LDH, and hemoglobin treated as continuous variables, values were summarized at each visit through Week 28 using mean, median, SD, minimum, and maximum, for both the ITT and PP populations. Mean changes from Baseline were analyzed using a restricted maximum likelihood (REML)-based repeated measures approach. Analyses included the fixed, categorical effect of visit as well as the continuous, fixed covariates of baseline value and the number of doses of eculizumab received through Week 8.

For the CKD stage, AKI stage, and MRS, the number and percent of patients within each category were presented at Baseline and each visit through Week 28. Also they were treated as an ordinal variable and shift tables were used to show the change in score from Baseline to Weeks 8, 16, and 28 for both ITT and PP patients. For MRS, the following 4 categories were used for the shift tables: (1) 0 or 1, (2) 2 or 3, (3) 4 or 5, and (4) 6. In addition, for each endpoint (CKD, AKI, and MRS), a shift table was presented for change from Baseline to the last observed value, and a Wilcoxon signed rank test was used to compare these 2 time points.

The number and percent of patients with overall normalization, complete hematologic response (Week 28 only), and who had thrombotic events were summarized along with 95% CIs at Weeks 8, 16, and 28 for both ITT and PP patients.

Kaplan-Meier estimation was used to summarize (1) the time-to-end of dialysis for those on dialysis at Baseline, (2) the time-to-end of PE/PI for those on PE/PI at Baseline, (3) the time-to-end of seizures for those having seizures at Baseline, and (4) the time-to-end of therapeutic coma for those in a therapeutic coma at Baseline, all from first dose up to Week 28 for both ITT and PP populations. The cumulative incidence was estimated using the CDF. Patients who were on either dialysis or PE/PI during the Baseline window, but who discontinued dialysis or PE/PI prior to Day 0 (which is the first dose of eculizumab) were excluded from these analyses, respectively.

Logistic regression was performed to explore the effect of various baseline factors, as well as certain dosing and dosing-related variables, on (1) creatinine normalization, (2) MRS normalization, and (3) overall normalization, for both the ITT and PP population at 3 study time points: Weeks 8, 16, and 28. A univariate logistic regression was performed for each of the following covariates on each of these 3 endpoints separately for each time point. Odds ratios and 95% CIs were presented to quantify the effects of the covariates on the endpoints.

The covariates modeled included:
Number of doses of eculizumab received through Week 8, treated as continuous (ITT population only)
Time from diarrhea onset to the initiation of eculizumab, in days (treated as continuous)
PE/PI at Baseline (yes/no)
Ever on PE/PI at Baseline (yes/no)
Number of days of PE/PI prior to the treatment of eculizumab, treated as continuous
Age at first infusion of eculizumab, treated as continuous
Gender (male/female)

STUDY PATIENTS Disposition of Patients

Inclusion Criteria Patient and/or legal guardian must be willing and able to give written informed consent. Adults, adolescents, or pediatric (≧2 months and ≧5 kg) patients. All of the following laboratory results:
STx+ or EHEC+ or Recent History of Bloody Diarrhea (tests pending), AND
Platelets≦150,000 or ≧25% decrease in 1 week or less, AND
Evidence of hemolysis (LDH>uln OR Schist OR Hapt<LLN).

Involvement of One or more of the following organs:
Kidney:
   Acute Kidney Injury I (AKI I or greater), plausibly related to the EHEC event and change in function within 48 hours:
1. increase in serum creatinine by ≧0.3 mg/dl, OR
2. increase in serum creatinine to ≧150% of the upper limit of normal, OR
3. urine output<0.5 mL/kg/>6 h)
   If continuous dialysis, then must be <1 week
   If continuous dialysis>1 week then must be biopsy within 48 hours demonstrating acute inflammation
Brain—one or more of the following (plausibly related to the EHEC event, with sign or symptom initiated no more than 1 week prior to enrollment, and evaluated by trained and certified neurology specialist)
   NIHSS≧1
   Mini-Mental-State Exam (MMSE) □□27
   Agitation with signs of anxiety or disorientation
   Hallucinations, psychosis
   Myoclonus
   Epileptic seizure
   Medically induced coma≦72 hours acceptable
Venous or Arterial Thrombosis plausibly related to the EHEC event and occurring no more than 2 weeks prior to initiation of eculizumab treatment

Exclusion Criteria
1. Known complement regulatory mutation or family history of complement regulatory mutation
2. Patients with ongoing sepsis defined as positive blood cultures within 7 days of the screening visit and not treated with antibiotics to which the organism is sensitive.
3. Pregnancy or lactation, except when physician determines that the benefit outweighs the risk.
4. Unresolved systemic meningococcal disease.
5. Any medical or psychological condition that, in the opinion of the investigator, could increase the patient's risk by participating in the study or confound the outcome of the study.
6. Hypersensitivity to eculizumab, to murine proteins or to one of the excipients.
7. Use of other experimental medicine/inclusion in other investigational intervention studies

One hundred ninety-eight patients were enrolled in the study. For efficacy and safety analyses, 198 patients received at least one dose of eculizumab and were included in the ITT population. The safety population was defined the same as the ITT population for this study. A total of 184/198 patients (93%) completed the study. Fourteen patients (7%) were withdrawn from the study. The primary reason for withdrawal was “Lost to Follow-Up.”

A total of 133 patients comprised the PP population. A total of 126/133 patients (95%) completed the study. Seven patients (5%) in the PP population were withdrawn from the study. The primary reason for withdrawal was “Lost to Follow-Up.”

TABLE 4
Patient Disposition (ITT/Safety and PP Populations)

N (%)

| | ITT/Safety | | | |
|---|---|---|---|---|
| Characteristic | Population | | PP Population | |
| Treated | 198 | (100) | 133 | (100) |
| Completed Study | 184 | (92.9) | 126 | (94.7) |
| Completed 8 weeks | 197 | (99.5) | 133 | (100) |
| Completed 16 weeks | 191 | (96.5) | 131 | (98.5) |
| Patients discontinued | 10 | (5.1) | 2 | (1.5) |
| Withdrawn from study | 14 | (7.1) | 7 | (5.3) |
| Investigator decision | 0 | (0.0) | 0 | (0.0) |
| Patient/parent decision | 3 | (1.5) | 2 | (1.5) |
| Lost to follow-up | 7 | (3.5) | 3 | (2.3) |
| Non-compliance with study | 0 | (0.0) | 0 | (0.0) |
| Protocol violation | 0 | (0.0) | 0 | (0.0) |
| Adverse event | 2 | (1.0) | 1 | (0.8) |
| Death | 0 | (0.0) | 0 | (0.0) |
| Pregnancy | 0 | (0.0) | 0 | (0.0) |
| Patient condition | 2 | (1.0) | 1 | (0.8) |
| Unknown | 0 | (0.0) | 0 | (0.0) |
| Other | 0 | (0.0) | 0 | (0.0) |
| No Reason Given | 0 | (0.0) | 0 | (0.0) |

A total of 180 patients (91%) in the ITT population met all enrollment criteria. Reasons for not meeting enrollment criteria for the remaining 18 patients included: abnormal laboratory studies for STEC, platelets, LDH, Schistocytes or haptoglobin (n=9); no kidney, brain or thrombosis involvement (n=4); and did not receive meningococcal vaccination (n=5).

Protocol Deviations

Protocol deviations that were noted in this study, as derived programmatically from the clinical database, are shown in Table 4. The most common protocol deviations included that the patient received less than the minimum number of eculizumab doses during the first 8 weeks of treatment (26%), MRS scoring was not done by a trained and certified neurologist (25%), missed supplemental eculizumab doses for PE/PI during treatment (22%) and had no positive STEC test (21%). Deviations associated with dosing made assessments of efficacy more difficult than expected; however, the data were not negatively impacted.

TABLE 5
Protocol Deviations (ITT Population)

| | ITT |
|---|---|
| | Population |
| Parameter | N = 198 |
| Less than minimum number of doses in 8 weeks | 51 (25.8) |
| Cumulative dose deviation | 39 (19.7) |
| Missed supplemental eculizumab dose for PE/PI | 43 (21.7) |
| during treatment | |
| Prohibited medication | 8 (4.0) |
| No positive STEC test | 40 (20.2) |
| Missed response on the primary endpoint | 0 (0.0) |
| Lost to follow-up | 7 (3.5) |
| MRS scoring not by trained and certified | 49 (24.7) |

Efficacy Evaluation

The primary analysis population for this study is the ITT population comprised of the 198 patients who received at least one dose of study drug. The PP population (n=133) was considered the secondary analysis population. All analyses were based on the totality of available data including the Screening Period, the 8 Week Treatment Period and Extension Treatment Period (initial 8 weeks, followed by additional eculizumab doses for up to a total of 16 weeks for selected patients based on physician decision, respectively), and at least the 12 Week Post-Treatment Period. Table 5 provides an overview of the number and percentage of patients in the ITT population who failed to meet 4 criteria qualifying them for inclusion in the PP population. The primary reason for exclusion from the PP analysis was not receiving the minimum number of doses in 8 weeks (51/198 patients; 26%). See Table 5.

TABLE 6
Summary of Patients Excluded from PP Analysis

Population (N = 198)

| | Yes n | No n |
|---|---|---|
| PP Population Criteria | (%) | (%) |
| Received at least the minimum number | 147 (74.2) | 51 (25.8) |
| No cumulative dose deviations | 159 (80.3) | 39 (19.7) |
| No prohibited medications | 190 (96.0) | 8 (4.0) |
| Met all inclusion/exclusion | 180 (90.9) | 18 (9.1) |
| criteria | | |

Demographic Characteristics

Demographic characteristics of the study populations are presented in Table 7. For both the ITT and PP populations, the median age was approximately 40 years (range, 8.3 to 84.5 years for the ITT population) and the majority were in the 18-<45 years age group (57% for the ITT population). All patients were Caucasian. A total of 9 pediatric patients participated in the trial. The majority of patients in both populations were female (>67%).

TABLE 7
Patient Demographic Characteristics

Summary Statistics

| | ITT | | | |
|---|---|---|---|---|
| | Population | | PP | |
| Characteristic | (N = 198) | | Population | |
| Mean Age, years (SD) | 42.1 | (17.1) | 43.5 | (16.4) |
| Median (Min; Max) | 39.3 | (8.3; 84.5) | 40.3 | (10.0; |
| Population age | | | | |
| category, n (%) | | | | |
| <18 years | 9 | (4.5) | 3 | (2.3) |
| 18-<45 years | 112 | (56.6) | 75 | (56.4) |
| 45-<65 years | 51 | (25.8) | 36 | (27.1) |
| ≧65 years | 26 | (13.1) | 19 | (14.3) |
| Sex, n (%) | | | | |
| Female | 142 | (71.7) | 90 | (67.7) |
| Male | 56 | (28.3) | 43 | (32.3) |

The most frequently reported (>50% of patients) concomitant medications by anatomical therapeutic category (ATC) and by WHO Drug Dictionary preferred term, 1 Mar. 2010 administered to patients in the ITT population were analgesics (86%, 171 patients) [paracetamol (63%, 124 patients]; antibacterials for systemic use (98%, 193 patients) [azithromycin 86%, 171 patients]; antiepileptics (58%, 114 patients); antihistamines (62%, 123 patients) [clemastine (56%, 111 patients]; antithrombotic agents (94%, 187 patients); blood substitutes and perfusion solutions (67%, 132 patients); calcium channel blockers (58%, 114 patients); corticosteroids for systemic use (60%, 119 patients); diuretics (63%, 124 patients); drugs for acid related disorders (88%, 175 patients) [pantoprazole sodium (61%, 121 patients), ranitidine hydrochloride (62%, 123 patients)]; drugs for functional gastrointestinal (GI) disorders (71%, 141 patients); mineral supplements (57%, 113 patients); psycholeptics (73%, 144 patients); and vaccines (99%; 195 patients) [meningococcal polysaccharide (98%, 194 patients)].

All but 43 patients received a meningococcal vaccine; nearly 50% of those vaccinated received a conjugated, quadrivalent formulation. A booster was received by 54% (106/198) of patients; 26/27 (96%) of the patients who received eculizumab beyond 8 weeks received a booster.

Efficacy Results

The primary analysis population for this study was the ITT population, which was comprised of the 198 patients who received at least one dose of study drug. The PP population (n=133) was considered the secondary analysis population. All patients enrolled in the study were Caucasian, and majority were female (72%). The mean age at Baseline was 42 years, with the majority of patients (57%) falling into the 18-<45 years-old age group. Nine patients were under the age of 18, 4 of whom were <12 years of age.

At Baseline, all but one of the patients enrolled were hospitalized. Greater than 75% of patients had both kidney and brain involvement. Greater than 21% of patients were receiving ventilator support, had evidence of neurologic sequelae based on a history of seizures at study entry, and had been placed in therapeutic comas. Over two-thirds (≧69%) of patients were receiving dialysis at Baseline, and >91% were receiving PE/PI.

The patients enrolling into the present study demonstrated a high proportion of involvement of one or more vital organs and also a high degree of organ morbidity, with an anticipated high mortality risk despite intensive supportive care. Of note as well, these patients exhibited either no improvement or worsening of TMA and organ function parameters in the days leading up to eculizumab treatment despite receiving multiple supportive care measures such as PE/PI, dialysis, antibiotics, strongly suggesting that for these patients, STEC-HUS was not a self-limiting disease. Specifically, at eculizumab initiation, >91% of patients were receiving PE/PI 88% and 97% of patients had thrombotic thrombocytopenia and microangiopathic hemolysis with elevated LDH, respectively. Importantly as well, 96% of patients had clinically important baseline kidney injury, 92% of patients had AKI 1 or greater, and 72% of patients were dialysis dependent. Similarly, despite best supportive care, at eculizumab initiation, 84% of study patients showed significant baseline neurologic involvement and in patients with available data, 98% of patients had MRS 2 or greater and 61% of patients had MRS 4-5, indicating a high degree of neurologic morbidity and anticipated mortality risk. Furthermore, at Baseline, approximately 25% of patients were receiving ventilator support, had recent history of seizures and had been placed in a therapeutic coma. Overall, 80% of patients in the trial had both brain and kidney vital organ involvement as manifestations of STEC-HUS.

Primary Endpoint

The primary endpoint for the study was defined as the improvement in systemic TMA and vital organ involvement at 8 weeks of treatment (complete plus partial responders). By Week 8, a total of 187/198 (94%) patients achieved at least a partial response, and a complete response was observed in 159/198 (80%) patients. By Week 28, additional patients had achieved a complete response, with the complete response rate increasing to 176/198 (89%). Using backwards stepwise regression, factors that were statistically significant predictors for patients having a CR at Week 8 included age at first dose and time from onset of diarrhea to the initiation of eculizumab, with younger patients and those dosed closer to the onset of diarrhea being more likely to experience a CR at Week 8. The factor that remained in the model as a predictor of CR+PR at Week 8 was duration of PE/PI prior to dosing with eculizumab, although it was not statistically significant. At Week 28, similar factors were determined to be statistically significant for response rates. The global assessment of efficacy revealed that 94% of patients achieved the primary endpoint of improvement in systemic TMA and vital organ involvement after Week 8. See Tables 8-10.

TABLE 8
End Point Values

| End point values | eculizumab | Per protocol |
|---|---|---|
| | | population |
| Subject group type | Reporting | Subject analysis |
| | group | set |
| Number of subjects | 198 | 133 |
| Units: Percentage of | | |
| patients | | |
| Number (confidence | | |
| interval 95%) | | |
| Complete response | 80.3 (0.741 | 82.7 (0.752 to |
| | to 0.856) | 0.887) |
| Partial response | 94.4 (0.903 | 92.5 (0.866 to |
| | to 0.972) | 0.963) |

TABLE 9
Primary: Improvement in systemic TMA and vital organ at Wk 28

| End point values | eculizumab | Per protocol |
|---|---|---|
| | | population |
| Subject group type | Reporting group | Subject analysis set |
| Number of subjects | 198 | 133 |
| analyzed | | |
| Units: Percentage of | | |
| patients | | |
| Number (confidence | | |
| interval 95%) | | |
| Complete response | 88.9 (0.837 to | 86.5 (0.795 to |
| | 0.929) | 0.918) |
| Partial response | 94.4 (0.903 to | 92.5 (0.866 to |
| | 0.972) | 0.963) |

TABLE 10
Primary: Improvement in systemic TMA and vital
organ at Wk 8 for Patients Dosed Beyond 8 Weeks

| End point values | eculizumab | Per protocol |
|---|---|---|
| | | population |
| Subject group type | Reporting | Subject analysis set |
| | group | |
| Number of subjects | 198 | 133 |
| analyzed | | |
| Units: Percentage of | | |
| patients | | |
| Number (confidence | | |
| interval 95%) | | |
| Complete response | 74.1 (0.537 to | 72.7 (0.498 to |
| | 0.889) | 0.893) |
| Partial response | 85.2 (0.663 to | 81.2 (0.597 to |
| | 0.958) | 0.948) |

Secondary Endpoints

Overall Effects

The proportion of patients achieving clinically important improvement increased over time to greater than 96% of patients who were affected at Baseline, for the global assessments of both neurological (146/152 patients) and renal (180/182 patients; 99%) function. No patients required new dialysis treatment after 14 days of eculizumab treatment. See Tables 10-14.

The proportion of patients in the ITT group who achieved overall normalization of hematologic, renal and neurologic parameters, reached 70% (137/197 patients; 95% CI, 0.6260, 0.7588) by Week 28. Subgroup analysis of overall normalization determined that gender (females vs. males) and age at first infusion of eculizumab were statistically significant at Week 8, suggesting that male patients and younger patients were more likely to achieve overall normalization by Week 8.

Clinically important improvement increased over time to greater than 96% of patients who were affected at Baseline, for the global assessments of both neurological and renal function. No patient required new dialysis treatment after 14 days of eculizumab treatment, and all but 2 patients were dialysis-free by Week 28.

Hematologic Effects

Hematologic normalization was achieved by greater than 90% of patients by Day 20. Normalization in platelet count, hemoglobin and LDH was rapid and determined to be statistically significant; no contributory covariates were identified in subgroup analysis. Platelet count improved dramatically and most rapidly after initiation of eculizumab. A rapid decrease in the number of TMA events also was noted, and greater than 95% of patients had achieved TMA event-free status by Day 13. Only 4 new thrombotic events occurred between Baseline and Week 28. See Table 15.

Renal Effects

The improvement in serum creatinine was dramatic and statistically significant at all analyzed time points. Subgroup analysis of creatinine normalization determined that gender (females vs. males) was the only categorical variable that showed a statistically significant effect at Weeks 8 and 28, suggesting that male patients were more likely to normalize.

Improvements in eGFR were also significant at all analyzed time points. One hundred percent of patients who had not been on dialysis at Baseline attained an increase in eGFR from Baseline ≧15 mL/min/1.73 m² by Day 56. Approximately 75% of all patients attained an eGFR≧60 mL/min/1.73 m², and approximately 30% of patients attained an eGFR of ≧90 mL/min/1.73 m² by the end of the study.

All but two patients (135/137, 99%) on dialysis at baseline discontinued dialysis by Week 28.

Shifts in CKD and AKI Stages from Baseline through the end of the study also showed statistically significant and clinically meaningful improvement. See Table 11.

Importantly, the improvement noted in serum creatinine and eGFR once receiving treatment with eculizumab was seen to be significantly better than the worsening noted prior to treatment.

Neurological Effects

Neurological normalization by Week 28 was reported in 91% (134/147) of patients, and subgroup analyses showed that the number of doses of eculizumab received through Week 8 and the age at the first dose of eculizumab at Weeks 8 and 28 were statistically significant. These analyses suggest that patients who received more eculizumab doses and those who were younger were more likely to normalize at the respective timepoints. Modified Rankin Scale (MRS) scores at Baseline indicated substantial significant neurological disability in almost all patients. By Week 28, 95% (123/130) of these patients had MRS scores indicating normalization of neurological disability after eculizumab treatment. Shifts in MRS scores from Baseline through the end of the study also showed statistically significant and clinically meaningful improvement. See Table 11.

All but one patient was seizure free by Week 8. One patient experienced a seizure on Day 80. Subsequently, all patients continued seizure-free. All but two patients discontinued AEDs by Week 28.

TABLE 11
Secondary: Global Assessment of Neurological Function

| End point values | eculizumab |
|---|---|
| Subject group type | Reporting group |
| Number of subjects | 198 |
| analyzed | |
| Units: Percentage of | |
| patients | |
| Number (confidence | |
| interval 95%) | |
| Week 8 | 88.8 (0.827 to |
| | 0.933) |
| Week 26 | 96.1 (0.916 to |
| | 0.985) |

TABLE 12
Secondary: Global Assessment of Renal Function

| End point values | eculizumab |
|---|---|
| Subject group type | Reporting group |
| Number of subjects | 198 |
| Units: Percentage of | |
| patients | |
| Number (confidence | |
| interval 95%) | |
| Week 8 | 96.2 (0.922 to |
| | 0.984) |
| Week 26 | 98.8 (0.961 to |
| | 0.999) |

TABLE 13
Secondary: New Ventilator Requirement

| End point values | eculizumab |
|---|---|
| Subject group type | Reporting group |
| Number of subjects | 198 |
| Units: percent | |
| Number (confidence | |
| interval 95%) | |
| Week 8 | 6.1 (0.032 to |
| | 0.103) |
| Week 26 | 6.1 (0.032 to |
| | 0.103) |

TABLE 14
Secondary: New Dialysis After Day 14 of eculizumab
treatment

| | |
|---|---|
| End point values | eculizumab |
| Subject group type | Reporting group |
| Number of subjects | 198 |
| analyzed | |
| Units: Percentage of | |
| patients | |
| Number (confidence | |
| interval 95%) | |
| Week 8 | 0 (0 to 0.067) |
| Week 26 | 0 (0 to 0.067) |

TABLE 15
Secondary: Hematological Normalization

| End point values | eculizumab |
|---|---|
| Subject group type | Reporting group |
| Number of subjects | 198 |
| analyzed | |
| Units: Percentage of | |
| patients | |
| Number (confidence | |
| interval 95%) | |
| Week 8 | 97 (0.935 to |
| | 0.989) |
| Week 26 | 98.5 (0.956 to |
| | 0.997) |

End point values are shown in Tables 16.

TABLE 16
Post-hoc: Hematological Normalization and No New
Organ Involvement

| End point values | eculizumab |
|---|---|
| Subject group type | Reporting group |
| Number of subjects | 198 |
| analyzed | |
| Units: Percentage of | |
| patients | |
| Number (confidence | |
| interval 95%) | |
| Week 8 | 91.4 (0.866 to |
| | 0.949) |
| Week 26 | 92.9 (0.884 to |
| | 0.961) |

Safety Results

The safety profile of eculizumab in patients with STEC-HUS was favorable and consistent with the safety profiles noted in prior aHUS and PNH eculizumab studies as well as for eculizumab overall. Adverse events (AEs) were reported in 196/198 (99%) patients, the majority of which were of mild to moderate severity. The most common individual AEs reported included headache (48%), hypertension (37%), alopecia (35%), peripheral edema (32%), nausea (31%), pleural effusion (24%) and vomiting (22%). In the majority of cases, AEs were considered to be related to the underlying disease.

Serious adverse events (SAEs) were noted in 33% of patients; there were a total of 123 events. The highest percentage of SAEs occurred in patients over the age of 45. The most common individual SAEs were convulsion (12%) and pneumonia (5%). No deaths occurred during the course of the study.

A total of 103 patients were reported to have an infection-related adverse event during the study. Sixteen of these patients experienced an infection reported as an SAE, and 9 of these patients had an SAE graded as severe in intensity. The most common infections by proportion of patients were nasopharyngitis (16%), urinary tract infections (11%) and pneumonia (10%); pneumonia also was the most common infection SAE (5%).

Adverse events deemed related (possibly or probably) to eculizumab (drug-related AEs) were reported in 79% of patients, the majority of which were moderate in severity. The most common individual drug-related events included alopecia (32%), headache (23%), fatigue (12%) and nausea (11%). Serious adverse events deemed possibly or probably related to eculizumab were reported in 19 out of 66 patients with SAEs (29%).

All but 3 patients received a meningococcal vaccine with nearly 50% having received a conjugated tetravalent vaccine. Fifty-four percent (106/198) of patients received a vaccine booster as well. Ninety-two percent of patients received concomitant macrolide antibiotics; the most common was azithromycin. Most of the antibiotics were administered during the first two weeks of eculizumab treatment initiation. No meningococcal infections were reported.

Complications were noted in two patients who were pregnant at Baseline but the patients delivered healthy children with no noted morbidity or defect.

In general, laboratory parameters that were abnormal at Baseline improved throughout the study, and no abnormalities emerged that were deemed related to study drug. Importantly, there was a statistically significant and clinically meaningful improvement in proteinuria from Baseline to the end of study.

Vital signs were largely unremarkable. Notably, the overall proportion of hypertensive patients diminished substantially between Baseline (60%) and Week 28 (24%).

A summary of patients with treatment-emergent adverse events (TEAEs) is provided in Table 17. Of the 198 patients evaluable for safety, 196 (99.0%) patients experienced at least one TEAE.

One hundred fifty-six (156) of 198 (79%) patients had a TEAE considered as least possibly related to study drug. Approximately one-third of patients (69 patients; 35%) had at least one severe TEAE in the study. There were 66 of 198 (33%) patients with at least one SAE, of whom 19 (10%) of 198 patients had drug-related SAEs considered at least possibly related to study drug. Ten (10) patients discontinued study drug because of adverse events. No patients experienced a TEAE associated with meningococcal infections.

TABLE 17
Summary of Patients with Treatment Emergent
Adverse Events (Safety Population)

| | All Patients |
|---|---|
| | (N = 198) |
| | N (%) |
| At least one TEAE | 196 (99.0) |
| At least one TEAE related to eculizumab^{a} | 156 (78.8) |
| At least one severe (Grade 3 or higher) TEAE | 69 (34.8) |
| At least one serious TEAE | 66 (33.3) |
| At least one serious TEAE related to eculizumab | 19 (9.6) |
| At least one TEAE leading to study drug | 10 (5.1) |
| At least one TEAE leading to study | 2 (1.0) |
| TEAE leading to death | 0 (0) |

^{a}Defined as possibly, probably or definitely related as assessed by the Principal Investigator

Adverse events by SOC reported in at least 20% of patients included nervous system disorders (147 of 198 patients, 74%), general disorders and administration site conditions (141 of 198 patients, 71%), gastrointestinal disorders (135 of 198 patients, 68%), infections and infestations (103 of 198 patients, 52%), skin and subcutaneous tissue disorders (102 of 198 patients, 52%), vascular disorders (100 of 198 patients, 51%), respiratory, thoracic and mediastinal disorders (99 of 198 patients, 50%), psychiatric disorders (91 of 198 patients, 46%), musculoskeletal and connective tissue disorders (73 of 198 patients, 37%), investigations (62 of 198 patients, 31%), cardiac disorders (48 of 198 patients, 24%), eye disorders (43 of 198 patients, 22%) and blood and lymphatic system disorders (41 of 198 patients, 21%). The most common infections reported were nasopharyngitis (31 of 198, 16%), urinary tract infections (22 of 198 patients, 11%) and pneumonia (19 of 198 patients, 10%). There were no meningococcal infections. All except 3 patients were vaccinated against meningococcal infection.

Overall, the most common events in descending order included headache (95 of 198 patients, 48%), hypertension (74 of 198 patients, 37%), alopecia (69 of 198 patients, 35%), peripheral edema (64 of 198 patients, 32%), nausea (61 of 198 patients, 31%), pleural effusion (48 of 198 patients, 24%) and vomiting (44 of 198 patients, 22%).

Conclusion

Uncontrolled complement system activation plays a critical role in patients with STEC-HUS, a rare, systemic disease associated with Shiga-toxin-producing bacteria, including Escherichia coli such as enterohemorrhagic E. coli (EHEC). The Shiga toxin produced by E. coli induces bloody diarrhea by damaging the lining of the large intestine. If the toxin is subsequently absorbed into the bloodstream, it also causes both direct alternative complement pathway activation, as well as impaired regulation of this pathway by direct binding to and inhibition of a key regulator of the alternative complement pathway: complement regulatory protein factor H. The dual activity of the Shiga toxin results in uncontrolled and excessive complement activation. This leads to inflammation and TMA with multi-organ damage, including kidney failure and severe neurological complications, resulting in life-threatening consequences.

At the end of May 2011, one of the largest outbreaks of EHEC infections occurred in Germany associated with progression to STEC-HUS in a large proportion of infected individuals. During this outbreak, the clinical presentation was severe with patients manifesting acute renal dysfunction and/or severe neurologic complications. Facing an unprecedented public health crisis and with anecdotal data in the literature regarding successful use of eculizumab, numerous physicians requested eculizumab for the management of their most severely ill STEC-HUS patients including those who were not responding to supportive care, symptomatic treatment and PE/PI.

The single-arm design of this study was considered the only trial design option in the context of the STEC-HUS crisis situation.

The duration of treatment for STEC-HUS patients with eculizumab was selected to ensure that complement inhibition would be sustained for at least 8 weeks as there is evidence that TMA can be present for several weeks after the initial EHEC infection. In addition, the protocol provided the option of an additional 8 weeks of treatment for patients who, in the opinion of the Investigator, may benefit from longer treatment duration because of residual abnormalities in kidney and/or CNS function present at 8 weeks.

All key endpoints for the trial were prospectively defined. The primary endpoint of this trial was chosen to enable an assessment of the overall clinical benefit of eculizumab, a composite endpoint of Improvement in Systemic TMA and Vital Organ Involvement (CR+PR) capturing hematologic improvement or normalization, clinically important improvement in all affected vital organs and no clinically important worsening in kidney, brain or thrombosis. Key secondary endpoints were chosen to enable an evaluation of the hypothesis that eculizumab can reverse the TMA process and improve renal (and neurological) function in patients with STEC-HUS. Since reversal of thrombocytopenia reflects a decrease in the TMA process and control of TMA, platelet count is a key clinical measure of the disease process and is recognized as a reliable measure of clinical disease activity, and thus of the efficacy of eculizumab.

Similarly, there were endpoints to evaluate the control of microangiopathic hemolysis (LDH, hemoglobin). Importantly as well several measures of renal function were used to determine improvement and normalization of renal function.

Endpoints and tools to assess neurological function were determined as part of the trial development discussions with the neurologist at the lead participating center in the trial. There are no tools validated for the occurrence of neurological signs and symptoms associated with STEC-HUS. After consideration of a number of scales, the MRS, validated for stroke, was recommended because neurologists and healthcare professionals have a great deal of experience with the tool and thus could readily implement. As a result, the data could be viewed as reliable. While the protocol specified neurologist evaluation, it was not always feasible at the participating centers given the crisis situation. At the same time, in lieu of a neurologist, a healthcare professional provided the assessment. The extensive prior experience with the MRS tool therefore was very useful in this crisis setting and allowed for assessment of neurological function in a uniform manner that would therefore have no impact on the interpretation of the results.

The patients enrolling in the present study demonstrated signs and symptoms of TMA, and a large proportion exhibited involvement of one or more vital organs as well as a high degree of organ morbidity, with an anticipated high mortality risk despite intensive supportive care. Of note as well, these patients exhibited either no improvement or worsening of TMA and organ function parameters in the days leading up to eculizumab treatment despite receiving multiple supportive care measures such as PE/PI, dialysis, antibiotics, strongly suggesting that for these patients, STEC-HUS was not a self-limiting disease. Specifically, at eculizumab initiation, 88% and 97% of patients had thrombotic thrombocytopenia and microangiopathic hemolysis, respectively, with elevated LDH. Importantly as well, 96% of patients had clinically important baseline kidney injury, 92% of patients had AKI 1 or greater, and 72% of patients were dialysis dependent. Similarly, despite available forms of supportive care, at eculizumab initiation, 84% of study patients showed significant Baseline neurologic involvement and in patients with available data, 98% of patients had MRS 2 or greater and 61% of patients had MRS 4-5, indicating a high degree of neurologic morbidity and anticipated mortality risk.

While EHEC/STEC infection was not always able to be verified by available assays in the context of the public health crisis, the clinical presentation of patients and epidemiological framework in which the patients presented provided for an overriding clinical diagnosis. Also, ad hoc analyses demonstrated that the results of various efficacy outcome measures are very similar between those patients with verified infection versus not.

All primary and secondary efficacy measures in this trial were achieved by the majority of patients at Week 8 and sustained at Week 28, and, where applicable, changes from Baseline in key measures of the TMA process and resulting vital organ damage (hematologic [platelet, LDH], kidney, brain) were statistically significant and represented clinically meaningful improvements. Taken together, these outcomes provide substantial evidence of efficacy for eculizumab in patients with STEC-HUS. Specifically,
For the primary endpoint of Improvement in Systemic TMA and Vital Organ Involvement, a total of 94% of patients achieved at least a partial response by Week 8 and a complete response was observed in 80%, with an additional 17 patients achieving a complete response by Week 28. Backwards stepwise regression analyses identified baseline factors that were statistically significant predictors for patients having a CR at Week 8. These factors included age at first dose and time from onset of diarrhea to the initiation of eculizumab, with younger patients and those dosed closer to the onset of diarrhea being more likely to experience a CR at Week 8. The factor that remained in the model as a predictor of CR+PR at Week 8 was duration of PE/PI prior to dosing with eculizumab, although it was not statistically significant.
At Week 28, similar factors were determined to be statistically significant for response rates.
Normalization in platelet count, hemoglobin and LDH was rapid and both statistically significant and clinically meaningful, compared to both baseline as well as the trajectory prior to baseline. The improvement in platelet count occurred rapidly after eculizumab was initiated. By the end of the first week, the median platelet count was in the normal range and 74% of patients normalized their platelet count. By the end of the second week, almost all patients had a normal platelet count and a few additional patients achieved platelet normalization in subsequent weeks, with 97% at Week 8 and 99% at Week 28. A rapid decrease in the number of TMA events also was noted, and greater than 95% of patients had achieved TMA event-free status by Day 13.
Overall, global renal function improvement was documented in 96% of patients at Week 8 and in 99% of patients by Week 28. Furthermore, renal function normalization was achieved in nearly all patients (94%) not on dialysis at Baseline by Week 8 that was sustained at Week 28, while it was achieved in 54% of patients on dialysis at Baseline at Week 8 and in 67% of patients at Week 28. Renal function improved significantly while on treatment compared to the trajectory prior to treatment.
During the first 8 weeks of eculizumab therapy, 89% of patients with documented brain involvement at Baseline achieved a clinically important neurologic improvement. At Baseline, 61% of patients with brain involvement and MRS evaluation exhibited MRS of 4-5 (moderate to severe disability). In contrast, at Week 8, 65% of patients with MRS score at this timepoint had achieved normal neurological function as assessed by MRS of 0-1. By Week 28, 95% of patients achieved a normal MRS. Importantly as well, all patients who had seizure reported at baseline became seizure-free by Week 28.

In contrast to the poor clinical outcomes in the pre-eculizumab period, rapid and profound clinical improvement was observed in hematologic and renal and neurologic function as soon as eculizumab therapy was initiated. With regard to hematologic parameters and consistent with what has been described in patients with aHUS, one of the earliest clinical improvements observed with initiation of eculizumab therapy was an increase in platelet count with normalization of platelet count occurring as early as one week. This clinical improvement is mechanistically linked to inhibition of terminal complement activity by eculizumab and initial control of the TMA process.

Important confirmation of the clinically meaningful and statistically significant improvement in kidney function with initiation of eculizumab treatment is further supported by improvement in other morbidities indicative of kidney injury or failure including hypertension and proteinuria.

At Baseline, 60% of patients were hypertensive; this proportion dramatically decreased to 22% at Week 8, and remained essentially unchanged at Week 28. Similarly, at Baseline, 60% of patients were receiving anti-hypertensive medications; this proportion decreased to 46% at Week 8 and further decreased considerably to 15% at Week 28 even as the proportion of patients no longer hypertensive was sustained. Similarly, shifts in proteinuria from Baseline to last observed value were statistically significant (P<0.0001). This improvement has medical implications as well given the recognized role of proteinuria as a measure of kidney damage.

In this severely ill population of STEC-HUS patients with one or more organ involvement, most (99%) patients experienced at least one TEAE, with AEs consistent with events that would be expected in the STEC-HUS setting. Approximately one-third of patients (33%) experienced at least one SAE; only 10% of the SAEs were deemed related to study drug. Importantly there were no deaths reported in this trial.

The safety profile of eculizumab observed in this trial is consistent with that observed in both PNH and aHUS, as well as for eculizumab overall. The most common TEAEs reported included headache (48%), hypertension (37%), alopecia (35%), peripheral edema (32%), nausea (31%), pleural effusion (24%) and vomiting (22%). The most common infections reported were nasopharyngitis (16%), urinary tract infections (11%) and pneumonia (10%). There were no meningococcal infections reported in this trial.

There are no approved therapies for patients with STEC-HUS, and available supportive therapies do not target the underlying pathophysiology of the disease. The population of patients in this trial had been receiving all available supportive therapies with the majority receiving PE/PI and antibiotics. In the days prior to receiving eculizumab, these patients were exhibiting no improvement or were worsening as evidenced by worsening renal function (creatinine, eGFR, requirement for dialysis), platelet count, LDH and hemoglobin. In addition, nearly two-thirds of the patients had substantial neurological morbidity with MRS of 4-5 and/or seizures. These characteristics indicate a severely ill population of STEC-HUS patients with involvement of one or more vital organs and at great risk for poor clinical outcomes.

The data from this trial demonstrate that in the setting of overall worsening TMA complications and deteriorating clinical condition despite multiple supportive care measures, treatment with eculizumab resulted in rapid and substantial improvement in all parameters with the vast majority of patients (94%) achieving the primary endpoint.

Importantly, the 3 piece linear repeated measures models demonstrate that the initiation of eculizumab in these STEC-HUS patients rapidly and profoundly changes the course of their disease. Within one week of the start of eculizumab, there was evidence of substantial inhibition of the TMA and inflammatory processes with platelet normalization and significant improvement in renal function followed then by dramatic improvement in neurological function. Altogether, the profound difference observed between the pre- and during-eculizumab periods demonstrates that the inhibition of uncontrolled complement activation with eculizumab leads to important clinical benefits in this population of patients with STEC-HUS and involvement of one or more vital organs. The totality and consistency of the rapid, sustained, and large magnitude outcomes reinforce the robustness of the treatment effect observed in this multi-center, single-arm clinical trial. Overall, eculizumab treatment of these STEC-HUS patients exhibited a favorable risk/benefit profile.

In summary, in this population of patients with STEC-HUS and involvement of one or more vital organs, treatment with eculizumab at doses prescribed in this study:
Was safe and well tolerated;
Showed that nearly all patients achieved the primary endpoint CR+PR/CR at Week 8 with additional patients achieving CR by Week 28;
Showed rapid improvement or normalization relative to Baseline of hematologic parameters in nearly all patients;
Showed statistically significant improvements relative to Baseline in renal function as assessed by serum creatinine and eGFR in patients not on dialysis at Baseline, normalization of renal function evaluated in all patients and dialysis status, with all but two patients on dialysis at Baseline becoming dialysis free
Showed statistically significant improvements relative to Baseline in neurological function including the majority of patients with Baseline neurological involvement achieving essentially normal neurological function with no persistent deficit (MRS score 0-1) by Week 8 and most patients achieving this normalization, and all patients achieving seizure free status by Week 28.

TABLE 18
List Of Abbreviations And Definitions Of Terms

| Abbreviation or | Definition |
|---|---|
| AE | Adverse event |
| AED | Anti-epileptic Drug |
| aHUS | Atypical hemolytic uremic syndrome |
| AKI | Acute Kidney Injury |
| ATC | Anatomical Therapeutic Chemical |
| CDF | Cumulative distribution function |
| CFH | Complement Factor H |
| CI | Confidence interval |
| CKD | Chronic kidney disease as defined by the |
| CNS | Central Nervous System |
| CR | Complete Response |
| CRF | Case report form |
| CRO | Contract Research Organization |
| CRP | C-reactive protein |
| CSR | Clinical Study Report |
| CT | Computed tomography |
| CTCAE | Common Terminology Criteria for Adverse |
| DGfN | Deutsche Gesellschaft fur Nephrologie |
| DGI | German Society for Infectiology |
| ECG | Electrocardiogram |
| eGFR | Estimated glomerular filtration rate |
| EHEC | Enterohemorrhagic Escherichia coli |
| EOS | End of study |
| ET | Early Termination |
| EU | European Union |
| FDA | Food and Drug Administration |
| FFP | Fresh frozen plasma |
| GCP | Good Clinical Practice |
| GI | Gastrointestinal |
| Hgb | Hemoglobin |
| HAHA | Human anti-human antibodies |
| Hapt | Haptoglobin |
| HPF | High Power Field |
| HUS | Hemolytic uremic syndrome |
| IAF | Informed assent Form |
| ICF | Informed consent Form |
| ICH | International Conference on Harmonisation |
| IEC | Independent ethics committee |
| IRB | Institutional Review Board |
| ITT | Intent-to-treat |
| IV | Intravenous |
| IVIg | Intravenous immunoglobulin |
| LDH | Lactate dehydrogenase |
| LLN | Lower limit of normal |
| MAVE | Major Adverse Vascular Events |
| Max | Maximum |
| MDRD | Modification of Diet in Renal Disease |
| MedDRA | Medical Dictionary for Regulatory Activities |
| Min | Minimum |

TABLE 19
Abbreviation of Special Terms

| Abbreviation or | Definition |
|---|---|
| MMSE | Mini-mental state examination |
| MRI | Magnetic Resonance Imaging |
| MRS | Modified Rankin Score |
| N | Number of patients |
| NIHSS | National Institutes of Health Stroke Scale |
| NYHA | New York Heart Association |
| OR | Odds ratio |
| PD | Pharmacodynamic |
| PDF | Portable document format |
| PE | Plasma exchange |
| PEI | Paul Ehrlich Institut |
| PE/PI (in conjunction | Plasma Exchange/Plasma Infusion |
| Pharsight | Pharsight - A Certara ™ Company |
| PI | Principal Investigator |
| PK | Pharmacokinetic |
| PNH | Paroxysmal nocturnal hemoglobinuria |
| PP | Per Protocol |
| PPH | Plasmapheresis |
| PR | Partial Response |
| PT | Plasma therapy (includes fresh frozen |
| | plasma plasmapheresis and plasma |
| PT (in conjunction | Preferred term |
| REML | Restricted Maximum Likelihood |
| SAE | Serious adverse event |
| SAP | Statistical analysis plan |
| Schist | Schistocytes |
| SD | Standard deviation |
| SOC | System Organ Class |
| STEC | Shiga toxin-producing Escherichia coli |
| STEC-HUS | Shiga toxin-producing Escherichia coli |
| STx | Shiga toxin |
| TEAE | Treatment-emergent adverse event |
| TMA | Thrombotic microangiopathy |
| ULN | Upper limit of normal |
| WBC | White Blood Cell |
| WHO | World Health Organization |

### Other Embodiments

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

TABLE 20
SOME NUCLEIC ACID AND AMINO ACID SEQUENCES

```
SEQ ID NO: 1
gat atc cag atg acc cag tcc ccg tcc tcc ctg tcc gcc tct gtg ggc
48
Asp Ile Gln Met Thr Gln Ser Pro Ser Ser Leu Ser Ala Ser Val Gly
1 5 10 15
gat agg gtc acc atc acc tgc ggc gcc agc gaa aac atc tat ggc gcg
96
Asp Arg Val Thr Ile Thr Cys Gly Ala Ser Glu Asn Ile Tyr Gly Ala
                        20 25 30
ctg aac tgg tat caa cag aaa ccc ggg aaa get ccg aag ctt ctg att
144
Leu Asn Trp Tyr Gln Gln Lys Pro Gly Lys Ala Pro Lys Leu Leu Ile
                35 40 45
tac ggt gcg acg aac ctg gca gat gga gtc cct tct cgc ttc tct gga
192
Tyr Gly Ala Thr Asn Leu Ala Asp Gly Val Pro Ser Arg Phe Ser Gly
        50 55 60
tcc ggc tcc gga acg gat ttc act ctg acc atc agc agt ctg cag cct
240
Ser Gly Ser Gly Thr Asp Phe Thr Leu Thr Ile Ser Ser Leu Gln Pro
65 70 75 80
gaa gac ttc get acg tat tac tgt cag aac gtt tta aat act ccg ttg
288
Glu Asp Phe Ala Thr Tyr Tyr Cys Gln Asn Val Leu Asn Thr Pro Leu
                                85 90 95
act ttc gga cag ggt acc aag gtg gaa ata aaa cgt act ggc ggt ggt
336
Thr Phe Gly Gln Gly Thr Lys Val Glu Ile Lys Arg Thr Gly Gly Gly
                        100 105 110
ggt tct ggt ggc ggt gga tct ggt ggt ggc ggt tct caa gtc caa ctg
384
Gly Ser Gly Gly Gly Gly Ser Gly Gly Gly Gly Ser Gln Val Gln Leu
                115 120 125
gtg caa tcc ggc gcc gag gtc aag aag cca ggg gcc tca gtc aaa gtg
432
Val Gln Ser Gly Ala Glu Val Lys Lys Pro Gly Ala Ser Val Lys Val
        130 135 140
tcc tgt aaa get agc ggc tat att ttt tct aat tat tgg att caa tgg
480
Ser Cys Lys Ala Ser Gly Tyr Ile Phe Ser Asn Tyr Trp Ile Gln Trp
145 150 155 160
gtg cgt cag gcc ccc ggg cag ggc ctg gaa tgg atg ggt gag atc tta
528
Val Arg Gln Ala Pro Gly Gln Gly Leu Glu Trp Met Gly Glu Ile Leu
                                165 170 175
ccg ggc tct ggt agc acc gaa tat acc gaa aat ttt aaa gac cgt gtt
576
Pro Gly Ser Gly Ser Thr Glu Tyr Thr Glu Asn Phe Lys Asp Arg Val
                        180 185 190
act atg acg cgt gac act tcg act agt aca gta tac atg gag ctc tcc
624
Thr Met Thr Arg Asp Thr Ser Thr Ser Thr Val Tyr Met Glu Leu Ser
                195 200 205
agc ctg cga tcg gag gac acg gcc gtc tat tat tgc gcg cgt tat ttt
672
Ser Leu Arg Ser Glu Asp Thr Ala Val Tyr Tyr Cys Ala Arg Tyr Phe
        210 215 220
ttt ggt tct agc ccg aat tgg tat ttt gat gtt tgg ggt caa gga acc
720
Phe Gly Ser Ser Pro Asn Trp Tyr Phe Asp Val Trp Gly Gln Gly Thr
225 230 235 240
ctg gtc act gtc tcg agc tga
741
Leu Val Thr Val Ser Ser
                                245
SEQ ID NO: 2
Asp Ile Gln Met Thr Gln Ser Pro Ser Ser Leu Ser Ala Ser Val Gly
1 5 10 15
Asp Arg Val Thr Ile Thr Cys Gly Ala Ser Glu Asn Ile Tyr Gly Ala
                        20 25 30
Leu Asn Trp Tyr Gln Gln Lys Pro Gly Lys Ala Pro Lys Leu Leu Ile
                35 40 45
Tyr Gly Ala Thr Asn Leu Ala Asp Gly Val Pro Ser Arg Phe Ser Gly
        50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Asn Val Leu Asn Thr Pro Leu
                                85 90 95
Thr Phe Gly Gln Gly Thr Lys Val Glu Ile Lys Arg Thr Gly Gly Gly
                        100 105 110
Gly Ser Gly Gly Gly Gly Ser Gly Gly Gly Gly Ser Gln Val Gln Leu
                115 120 125
Val Gln Ser Gly Ala Glu Val Lys Lys Pro Gly Ala Ser Val Lys Val
        130 135 140
Ser Cys Lys Ala Ser Gly Tyr Ile Phe Ser Asn Tyr Trp Ile Gln Trp
145 150 155 160
Val Arg Gln Ala Pro Gly Gln Gly Leu Glu Trp Met Gly Glu Ile Leu
                                165 170 175
Pro Gly Ser Gly Ser Thr Glu Tyr Thr Glu Asn Phe Lys Asp Arg Val
                        180 185 190
Thr Met Thr Arg Asp Thr Ser Thr Ser Thr Val Tyr Met Glu Leu Ser
                195 200 205
Ser Leu Arg Ser Glu Asp Thr Ala Val Tyr Tyr Cys Ala Arg Tyr Phe
        210 215 220
Phe Gly Ser Ser Pro Asn Trp Tyr Phe Asp Val Trp Gly Gln Gly Thr
225 230 235 240
Leu Val Thr Val Ser Ser
                                245
SEQ ID NO: 3
Gly Gly Gly Gly Ser Gly Gly Gly Gly Ser Gly Gly Gly Gly Ser
SEQ ID NO: 4
Met Gly Leu Leu Gly Ile Leu Cys Phe Leu Ile Phe Leu Gly Lys Thr
1 5 10 15
Trp Gly Gln Glu Gln Thr Tyr Val Ile Ser Ala Pro Lys Ile Phe Arg
                        20 25 30
Val Gly Ala Ser Glu Asn Ile Val Ile Gln Val Tyr Gly Tyr Thr Glu
                35 40 45
Ala Phe Asp Ala Thr Ile Ser Ile Lys Ser Tyr Pro Asp Lys Lys Phe
        50 55 60
Ser Tyr Ser Ser Gly His Val His Leu Ser Ser Glu Asn Lys Phe Gln
65 70 75 80
Asn Ser Ala Ile Leu Thr Ile Gln Pro Lys Gln Leu Pro Gly Gly Gln
                                85 90 95
Asn Pro Val Ser Tyr Val Tyr Leu Glu Val Val Ser Lys His Phe Ser
                        100 105 110
Lys Ser Lys Arg Met Pro Ile Thr Tyr Asp Asn Gly Phe Leu Phe Ile
                115 120 125
His Thr Asp Lys Pro Val Tyr Thr Pro Asp Gln Ser Val Lys Val Arg
        130 135 140
Val Tyr Ser Leu Asn Asp Asp Leu Lys Pro Ala Lys Arg Glu Thr Val
145 150 155 160
Leu Thr Phe Ile Asp Pro Glu Gly Ser Glu Val Asp Met Val Glu Glu
                                165 170 175
Ile Asp His Ile Gly Ile Ile Ser Phe Pro Asp Phe Lys Ile Pro Ser
                        180 185 190
Asn Pro Arg Tyr Gly Met Trp Thr Ile Lys Ala Lys Tyr Lys Glu Asp
                195 200 205
Phe Ser Thr Thr Gly Thr Ala Tyr Phe Glu Val Lys Glu Tyr Val Leu
        210 215 220
Pro His Phe Ser Val Ser Ile Glu Pro Glu Tyr Asn Phe Ile Gly Tyr
225 230 235 240
Lys Asn Phe Lys Asn Phe Glu Ile Thr Ile Lys Ala Arg Tyr Phe Tyr
                                245 250 255
Asn Lys Val Val Thr Glu Ala Asp Val Tyr Ile Thr Phe Gly Ile Arg
                        260 265 270
Glu Asp Leu Lys Asp Asp Gln Lys Glu Met Met Gln Thr Ala Met Gln
                275 280 285
Asn Thr Met Leu Ile Asn Gly Ile Ala Gln Val Thr Phe Asp Ser Glu
        290 295 300
Thr Ala Val Lys Glu Leu Ser Tyr Tyr Ser Leu Glu Asp Leu Asn Asn
305 310 315 320
Lys Tyr Leu Tyr Ile Ala Val Thr Val Ile Glu Ser Thr Gly Gly Phe
                                325 330 335
Ser Glu Glu Ala Glu Ile Pro Gly Ile Lys Tyr Val Leu Ser Pro Tyr
                        340 345 350
Lys Leu Asn Leu Val Ala Thr Pro Leu Phe Leu Lys Pro Gly Ile Pro
                355 360 365
Tyr Pro Ile Lys Val Gln Val Lys Asp Ser Leu Asp Gln Leu Val Gly
        370 375 380
Gly Val Pro Val Ile Leu Asn Ala Gln Thr Ile Asp Val Asn Gln Glu
385 390 395 400
Thr Ser Asp Leu Asp Pro Ser Lys Ser Val Thr Arg Val Asp Asp Gly
                                405 410 415
Val Ala Ser Phe Val Leu Asn Leu Pro Ser Gly Val Thr Val Leu Glu
                        420 425 430
Phe Asn Val Lys Thr Asp Ala Pro Asp Leu Pro Glu Glu Asn Gln Ala
                435 440 445
Arg Glu Gly Tyr Arg Ala Ile Ala Tyr Ser Ser Leu Ser Gln Ser Tyr
        450 455 460
Leu Tyr Ile Asp Trp Thr Asp Asn His Lys Ala Leu Leu Val Gly Glu
465 470 475 480
His Leu Asn Ile Ile Val Thr Pro Lys Ser Pro Tyr Ile Asp Lys Ile
                                485 490 495
Thr His Tyr Asn Tyr Leu Ile Leu Ser Lys Gly Lys Ile Ile His Phe
                        500 505 510
Gly Thr Arg Glu Lys Phe Ser Asp Ala Ser Tyr Gln Ser Ile Asn Ile
                515 520 525
Pro Val Thr Gln Asn Met Val Pro Ser Ser Arg Leu Leu Val Tyr Tyr
        530 535 540
Ile Val Thr Gly Glu Gln Thr Ala Glu Leu Val Ser Asp Ser Val Trp
545 550 555 560
Leu Asn Ile Glu Glu Lys Cys Gly Asn Gln Leu Gln Val His Leu Ser
                                565 570 575
Pro Asp Ala Asp Ala Tyr Ser Pro Gly Gln Thr Val Ser Leu Asn Met
                        580 585 590
Ala Thr Gly Met Asp Ser Trp Val Ala Leu Ala Ala Val Asp Ser Ala
                595 600 605
Val Tyr Gly Val Gln Arg Gly Ala Lys Lys Pro Leu Glu Arg Val Phe
        610 615 620
Gln Phe Leu Glu Lys Ser Asp Leu Gly Cys Gly Ala Gly Gly Gly Leu
625 630 635 640
Asn Asn Ala Asn Val Phe His Leu Ala Gly Leu Thr Phe Leu Thr Asn
                                645 650 655
Ala Asn Ala Asp Asp Ser Gln Glu Asn Asp Glu Pro Cys Lys Glu Ile
                        660 665 670
Leu Arg Pro Arg Arg Thr Leu Gln Lys Lys Ile Glu Glu Ile Ala Ala
                675 680 685
Lys Tyr Lys His Ser Val Val Lys Lys Cys Cys Tyr Asp Gly Ala Cys
        690 695 700
Val Asn Asn Asp Glu Thr Cys Glu Gln Arg Ala Ala Arg Ile Ser Leu
705 710 715 720
Gly Pro Arg Cys Ile Lys Ala Phe Thr Glu Cys Cys Val Val Ala Ser
                                725 730 735
Gln Leu Arg Ala Asn Ile Ser His Lys Asp Met Gln Leu Gly Arg Leu
                        740 745 750
His Met Lys Thr Leu Leu Pro Val Ser Lys Pro Glu Ile Arg Ser Tyr
                755 760 765
Phe Pro Glu Ser Trp Leu Trp Glu Val His Leu Val Pro Arg Arg Lys
        770 775 780
Gln Leu Gln Phe Ala Leu Pro Asp Ser Leu Thr Thr Trp Glu Ile Gln
785 790 795 800
Gly Ile Gly Ile Ser Asn Thr Gly Ile Cys Val Ala Asp Thr Val Lys
                                805 810 815
Ala Lys Val Phe Lys Asp Val Phe Leu Glu Met Asn Ile Pro Tyr Ser
                        820 825 830
Val Val Arg Gly Glu Gln Ile Gln Leu Lys Gly Thr Val Tyr Asn Tyr
                835 840 845
Arg Thr Ser Gly Met Gln Phe Cys Val Lys Met Ser Ala Val Glu Gly
        850 855 860
Ile Cys Thr Ser Glu Ser Pro Val Ile Asp His Gln Gly Thr Lys Ser
865 870 875 880
Ser Lys Cys Val Arg Gln Lys Val Glu Gly Ser Ser Ser His Leu Val
                                885 890 895
Thr Phe Thr Val Leu Pro Leu Glu Ile Gly Leu His Asn Ile Asn Phe
                        900 905 910
Ser Leu Glu Thr Trp Phe Gly Lys Glu Ile Leu Val Lys Thr Leu Arg
                915 920 925
Val Val Pro Glu Gly Val Lys Arg Glu Ser Tyr Ser Gly Val Thr Leu
        930 935 940
Asp Pro Arg Gly Ile Tyr Gly Thr Ile Ser Arg Arg Lys Glu Phe Pro
945 950 955 960
Tyr Arg Ile Pro Leu Asp Leu Val Pro Lys Thr Glu Ile Lys Arg Ile
                                965 970 975
Leu Ser Val Lys Gly Leu Leu Val Gly Glu Ile Leu Ser Ala Val Leu
                        980 985 990
Ser Gln Glu Gly Ile Asn Ile Leu Thr His Leu Pro Lys Gly Ser Ala
                995 1000 1005
Glu Ala Glu Leu Met Ser Val Val Pro Val Phe Tyr Val Phe His
        1010 1015 1020
Tyr Leu Glu Thr Gly Asn His Trp Asn Ile Phe His Ser Asp Pro
        1025 1030 1035
Leu Ile Glu Lys Gln Lys Leu Lys Lys Lys Leu Lys Glu Gly Met
        1040 1045 1050
Leu Ser Ile Met Ser Tyr Arg Asn Ala Asp Tyr Ser Tyr Ser Val
        1055 1060 1065
Trp Lys Gly Gly Ser Ala Ser Thr Trp Leu Thr Ala Phe Ala Leu
        1070 1075 1080
Arg Val Leu Gly Gln Val Asn Lys Tyr Val Glu Gln Asn Gln Asn
        1085 1090 1095
Ser Ile Cys Asn Ser Leu Leu Trp Leu Val Glu Asn Tyr Gln Leu
        1100 1105 1110
Asp Asn Gly Ser Phe Lys Glu Asn Ser Gln Tyr Gln Pro Ile Lys
        1115 1120 1125
Leu Gln Gly Thr Leu Pro Val Glu Ala Arg Glu Asn Ser Leu Tyr
        1130 1135 1140
Leu Thr Ala Phe Thr Val Ile Gly Ile Arg Lys Ala Phe Asp Ile
        1145 1150 1155
Cys Pro Leu Val Lys Ile Asp Thr Ala Leu Ile Lys Ala Asp Asn
        1160 1165 1170
Phe Leu Leu Glu Asn Thr Leu Pro Ala Gln Ser Thr Phe Thr Leu
        1175 1180 1185
Ala Ile Ser Ala Tyr Ala Leu Ser Leu Gly Asp Lys Thr His Pro
        1190 1195 1200
Gln Phe Arg Ser Ile Val Ser Ala Leu Lys Arg Glu Ala Leu Val
        1205 1210 1215
Lys Gly Asn Pro Pro Ile Tyr Arg Phe Trp Lys Asp Asn Leu Gln
        1220 1225 1230
His Lys Asp Ser Ser Val Pro Asn Thr Gly Thr Ala Arg Met Val
        1235 1240 1245
Glu Thr Thr Ala Tyr Ala Leu Leu Thr Ser Leu Asn Leu Lys Asp
        1250 1255 1260
Ile Asn Tyr Val Asn Pro Val Ile Lys Trp Leu Ser Glu Glu Gln
        1265 1270 1275
Arg Tyr Gly Gly Gly Phe Tyr Ser Thr Gln Asp Thr Ile Asn Ala
        1280 1285 1290
Ile Glu Gly Leu Thr Glu Tyr Ser Leu Leu Val Lys Gln Leu Arg
        1295 1300 1305
Leu Ser Met Asp Ile Asp Val Ser Tyr Lys His Lys Gly Ala Leu
        1310 1315 1320
His Asn Tyr Lys Met Thr Asp Lys Asn Phe Leu Gly Arg Pro Val
        1325 1330 1335
Glu Val Leu Leu Asn Asp Asp Leu Ile Val Ser Thr Gly Phe Gly
        1340 1345 1350
Ser Gly Leu Ala Thr Val His Val Thr Thr Val Val His Lys Thr
        1355 1360 1365
Ser Thr Ser Glu Glu Val Cys Ser Phe Tyr Leu Lys Ile Asp Thr
        1370 1375 1380
Gln Asp Ile Glu Ala Ser His Tyr Arg Gly Tyr Gly Asn Ser Asp
        1385 1390 1395
Tyr Lys Arg Ile Val Ala Cys Ala Ser Tyr Lys Pro Ser Arg Glu
        1400 1405 1410
Glu Ser Ser Ser Gly Ser Ser His Ala Val Met Asp Ile Ser Leu
        1415 1420 1425
Pro Thr Gly Ile Ser Ala Asn Glu Glu Asp Leu Lys Ala Leu Val
        1430 1435 1440
Glu Gly Val Asp Gln Leu Phe Thr Asp Tyr Gln Ile Lys Asp Gly
        1445 1450 1455
His Val Ile Leu Gln Leu Asn Ser Ile Pro Ser Ser Asp Phe Leu
        1460 1465 1470
Cys Val Arg Phe Arg Ile Phe Glu Leu Phe Glu Val Gly Phe Leu
        1475 1480 1485
Ser Pro Ala Thr Phe Thr Val Tyr Glu Tyr His Arg Pro Asp Lys
        1490 1495 1500
Gln Cys Thr Met Phe Tyr Ser Thr Ser Asn Ile Lys Ile Gln Lys
        1505 1510 1515
Val Cys Glu Gly Ala Ala Cys Lys Cys Val Glu Ala Asp Cys Gly
        1520 1525 1530
Gln Met Gln Glu Glu Leu Asp Leu Thr Ile Ser Ala Glu Thr Arg
        1535 1540 1545
Lys Gln Thr Ala Cys Lys Pro Glu Ile Ala Tyr Ala Tyr Lys Val
        1550 1555 1560
Ser Ile Thr Ser Ile Thr Val Glu Asn Val Phe Val Lys Tyr Lys
        1565 1570 1575
Ala Thr Leu Leu Asp Ile Tyr Lys Thr Gly Glu Ala Val Ala Glu
        1580 1585 1590
Lys Asp Ser Glu Ile Thr Phe Ile Lys Lys Val Thr Cys Thr Asn
        1595 1600 1605
Ala Glu Leu Val Lys Gly Arg Gln Tyr Leu Ile Met Gly Lys Glu
        1610 1615 1620
Ala Leu Gln Ile Lys Tyr Asn Phe Ser Phe Arg Tyr Ile Tyr Pro
        1625 1630 1635
Leu Asp Ser Leu Thr Trp Ile Glu Tyr Trp Pro Arg Asp Thr Thr
        1640 1645 1650
Cys Ser Ser Cys Gln Ala Phe Leu Ala Asn Leu Asp Glu Phe Ala
        1655 1660 1665
Glu Asp Ile Phe Leu Asn Gly Cys
        1670 1675
SEQ ID NO: 5
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKS
LSLSLGK
SEQ ID NO: 6
DIQMTQSPSSLSASVGDRVTITCGASENIYGALNWYQQKPGKAPKLLIYGATNLADGVPSRFS
GSGSGTDFTLTISSLQPEDFATYYCQNVLNTPLTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
heavy chain (g_{2/4}) (448 amino acids)
SEQ ID NO: 7
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVLHEALHSHYTQKS
LSLSLGK
light chain: (Kappa) (214 amino acids)
SEQ ID NO: 8
DIQMTQSPSSLSASVGDRVTITCGASENIYGALNWYQQKPGKAPKLLIYGATNLADGVPSRFS
GSGSGTDFTLTISSLQPEDFATYYCQNVLNTPLTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
SEQ ID NO: 9
GYIFSNYWIQ
SEQ ID NO: 10
EILPGSGSTEYTENFKD
SEQ ID NO: 11
YFFGSSPNWYFDV
SEQ ID NO: 12
GASENIYGALN
SEQ ID NO: 13
GATNLAD
SEQ ID NO: 14
QNVLNTPLT
SEQ ID NO: 15
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSS
SEQ ID NO: 16
DIQMTQSPSSLSASVGDRVTITCGASENIYGALNWYQQKPGKAPKLLIYGATNLADGVPSRFS
GSGSGTDFTLTISSLQPEDFATYYCQNVLNTPLTFGQGTKVEIK
amino acid sequence of heavy chain constant region of eculizumab
SEQ ID NO: 23
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHY
TQKSLSLSLGK
amino acid sequence of heavy chain variable region of BNJ441
antibody
SEQ ID NO: 24
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSS
amino acid sequence of heavy chain constant region of BNJ441
antibody
SEQ ID NO: 25
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVLHEALHSHY
TQKSLSLSLGK
amino acid sequence of IgG2 heavy chain constant region
variant comprising YTE substitutions
SEQ ID NO: 26
ASTKGPSVFPLAPCSRSTSESTAALGCLVKDYFPEPVTVSWNSGALTSGVHTFPAVLQSSGLY
SLSSVVTVTSSNFGTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQFNSTFRVVSVLTVVH
QDWLNGKEYKCKVSNKGLPAPIEKTISKTKGQPREPQVYTLPPSREEMTKNQVSLTCLVKGFY
PSDIAVEWESNGQPENNYKTTPPMLDSDGSFFLYSKLTVDKSRWQQGNVFSCSVMHEALHNHY
TQKSLSLSPGK
amino acid sequence of entire heavy chain of eculizumab
variant comprising heavy chain constant region depicted in
SEQ ID NO: 26 (above)
SEQ ID NO: 27
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVTSSNFGTQTYTCNVDHKPSNTKVDKTVERKCCVECPPCPAPPVAGPSVFLFPPKPKDTL
YITREPEVTCVVVDVSHEDPEVQFNWYVDGMEVHNAKTKPREEQFNSTFRVVSVLTVVHQDWL
NGKEYKCKVSNKGLPAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCSVMHEALHNHYTQKS
LSLSPGK
amino acid sequence of light chain CDR1 of eculizumab (as
defined under Kabat definition) with glycine to histidine
substitution at position 8 relative to SEQ ID NO: 12
SEQ ID NO: 28
GASENIYHALN
depicts amino acid sequence of heavy chain CDR2 of
eculizumab in which serine at position 8 relative to
SEQ ID NO: 10 is substituted with histidine
SEQ ID NO: 29
EILPGSGHTEYTENFKD
amino acid sequence of “FLAG” tag
SEQ ID NO: 30
DYKDDDDK
polyhistidine sequence commonly used as antigenic tag.
SEQ ID NO: 31
HHHHHH
amino acid sequence of hemagglutinin tag.
SEQ ID NO: 32
YPYDVPDYA
amino acid sequence of heavy chain CDR1 of eculizumab in
which tyrosine at position 2 (relative to SEQ ID NO: 9)
is substituted with histidine
SEQ ID NO: 33
GHIFSNYWIQ
```

1. A method of treating a complement mediated disorder caused by an infectious agent in a human patient comprising administering an effective amount of a polypeptide inhibitor of human complement C5 protein to the human patient.

2. The method of claim 1, wherein the infectious agent is selected from the group consisting of virus, bacteria, protozoa, fungi, prion and worm.

3. The method of claim 1, wherein the infectious agent is a virus that can cause hemorrhagic fever in the patient.

4. (canceled)

5. The method of claim 1, wherein the complement mediated disorder is sepsis.

6. (canceled)

7. The method of claim 1, wherein the infectious agent is a virus that can cause VHF or the complement mediated disorder is sepsis.

8. The method of claim 1, wherein the polypeptide inhibitor is a monoclonal antibody.

9. The method of claim 1, wherein the polypeptide inhibitor comprises a variable region of an antibody.

10. The method of claim 1, wherein the polypeptide inhibitor is eculizumab or an eculizumab variant, or antigen-binding fragment of either.

11-13. (canceled)

14. The method of claim 3, wherein the virus is a filovirus and wherein the filovirus is an Ebola virus.

15. The method of claim 1, further comprising administering a second therapeutic agent to the patient.

16-23. (canceled)

24. The method of claim 1, wherein the polypeptide inhibitor of complement C5 is a polypeptide comprising one or more of the amino acid sequence depicted in SEQ ID NOs:1-3, 5-8, or 24, 26-33, or an antigen binding fragment of any of the above.

25. The method of claim 1, wherein the polypeptide inhibitor of complement C5 is a polypeptide comprising one or more of the amino acid sequence depicted in SEQ ID NOs:9-16.

26. (canceled)

27. A method of treating a complement mediated disorder caused by an infectious agent in a human patient, comprising administering an effective amount of an anti-C5 antibody, or antigen binding fragment thereof, to the patient, wherein the complement mediated disorder is Shiga toxin-producing E. coli hemolytic uremic syndrome (STEC-HUS), wherein the method comprises an administration cycle comprising an induction phase followed by a maintenance phase, wherein:
the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or
the anti-C5 antibody, or antigen binding fragment thereof, is administered during the induction phase at a dose of 600 mg weekly for 1 week, starting at day 0, and is administered during the maintenance phase at a dose of 600 mg every week; or
the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.

28. The method of claim 27, wherein the anti-C5 antibody, or antigen binding fragment thereof, comprises CDR1, CDR2, and CDR3 heavy chain sequences as set forth in SEQ ID NOs:33, 29, and 11, respectively, and CDR1, CDR2, and CDR3 light chain sequences as set forth in SEQ ID NOs:12, 13, and 14, respectively.

29. (canceled)

30. The method of claim 27, wherein the anti-C5 antibody, or antigen binding fragment thereof, comprises a heavy chain variable region sequence as set forth in SEQ ID NO:15, and light chain variable region sequence as set forth in SEQ ID NO:16.

31. The method of claim 27, wherein the anti-C5 antibody, or antigen-binding fragment thereof, comprises a heavy chain variable region depicted in SEQ ID NO:24 and a light chain variable region depicted in SEQ ID NO:16.

32. (canceled)

33. The method of claim 27, wherein the anti-C5 antibody, or antigen binding fragment thereof, comprises a heavy chain sequence as set for in SEQ ID NO:5, and light chain sequence as set forth in SEQ ID NO:6 or SEQ ID NO:8.

34. The method of claim 27, wherein the antibody, or antigen-binding fragment thereof, comprises a heavy chain sequence depicted in SEQ ID NO:7 and a light chain sequence depicted in SEQ ID NO:6 or SEQ ID NO:8.

35-57. (canceled)

58. A kit for treating Shiga toxin-producing E. coli hemolytic uremic syndrome (STEC-HUS) in a human patient, the kit comprising:
(a) a dose of an anti-C5 antibody, or antigen binding fragment thereof; and
(b) Instructions for using the anti-C5 antibody, or antigen binding fragment thereof, in the method of claim 27.

59. An anti-C5 antibody, or antigen binding fragment thereof, comprising CDR1, CDR2 and CDR3 domains of the heavy chain variable region from the sequence set forth in SEQ ID NO:15 or SEQ ID NO:24, and CDR1, CDR2 and CDR3 domains of the light chain variable region from the sequence set forth in SEQ ID NO:16, for administration in a cycle comprising an induction phase followed by a maintenance phase, wherein:
the anti-C5 antibody, or antigen binding fragment thereof, is administered during the induction phase at a dose of 900 mg weekly for 4 weeks, starting at day 0, and is administered during the maintenance phase at a dose of 1200 mg in week 5 and then 1200 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 900 mg in week 3, and then 900 mg every two weeks; or
the anti-C5 antibody, or antigen binding fragment thereof, is administered during the induction phase at a dose of 600 mg weekly for 2 weeks, starting at day 0, and is administered during the maintenance phase at a dose of 600 mg in week 3, and then 600 mg every two weeks; or
the anti-C5 antibody, or antigen binding fragment thereof, is administered during the induction phase at a dose of 600 mg weekly for 1 week, starting at day 0, and is administered during the maintenance phase at a dose of 600 mg every week; or
the anti-C5 antibody, or antigen binding fragment thereof, is administered during the induction phase at a dose of 300 mg weekly for 1 week, starting at day 0, and is administered during the maintenance phase at a dose of 300 mg at week 2 and then every 3 weeks.


SEQUENCE LISTING (33 sequences)

SEQ ID NO: 1 - DNA, 741 bp, Artificial Sequence
  Description of Artificial Sequence Synthetic polynucleotide
gatatccagatgacccagtccccgtcctccctgtccgcctctgtgggcgatagggtcaccatcacctgcggcgccagcgaaaacatctatggcgcgctgaactggtatcaacagaaacccgggaaagctccgaagcttctgatttacggtgcgacgaacctggcagatggagtcccttctcgcttctctggatccggctccggaacggatttcactctgaccatcagcagtctgcagcctgaagacttcgctacgtattactgtcagaacgttttaaatactccgttgactttcggacagggtaccaaggtggaaataaaacgtactggcggtggtggttctggtggcggtggatctggtggtggcggttctcaagtccaactggtgcaatccggcgccgaggtcaagaagccaggggcctcagtcaaagtgtcctgtaaagctagcggctatattttttctaattattggattcaatgggtgcgtcaggcccccgggcagggcctggaatggatgggtgagatcttaccgggctctggtagcaccgaatataccgaaaattttaaagaccgtgttactatgacgcgtgacacttcgactagtacagtatacatggagctctccagcctgcgatcggaggacacggccgtctattattgcgcgcgttatttttttggttctagcccgaattggtattttgatgtttggggtcaaggaaccctggtcactgtctcgagctga

SEQ ID NO: 2 - protein, 246 aa, Artificial Sequence
  Description of Artificial Sequence Synthetic polypeptide
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTGGGGSGGGGSGGGGSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSS

SEQ ID NO: 3 - protein, 15 aa, Artificial Sequence
  Description of Artificial Sequence Synthetic peptide
GGGGSGGGGSGGGGS

SEQ ID NO: 4 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVILNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGIGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 5 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 214 aa, Artificial Sequence [sequence omitted]

SEQ ID NO: 7 - protein, 448 aa, Artificial Sequence
  Description of Artificial Sequence Synthetic polypeptide
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 8 - protein, 214 aa, Artificial Sequence [sequence omitted]

SEQ ID NO: 9 - protein, 10 aa, Artificial Sequence
  Description of Artificial Sequence Synthetic peptide
GYIFSNYWIQ

SEQ ID NO: 10 - protein, 17 aa, Artificial Sequence
  Description of Artificial Sequence Synthetic peptide
EILPGSGSTEYTENFKD

SEQ ID NO: 11 - protein, 13 aa, Artificial Sequence
  Description of Artificial Sequence Synthetic peptide
YFFGSSPNWYFDV

SEQ ID NO: 12 - protein, 11 aa, Artificial Sequence
  Description of Artificial Sequence Synthetic peptide
GASENIYGALN

SEQ ID NO: 13 - protein, 7 aa, Artificial Sequence
  Description of Artificial Sequence Synthetic peptide
GATNLAD

SEQ ID NO: 14 - protein, 9 aa, Artificial Sequence
  Description of Artificial Sequence Synthetic peptide
QNVLNTPLT

SEQ ID NO: 15 - protein, 122 aa, Artificial Sequence [sequence omitted]

SEQ ID NO: 16 - protein, 107 aa, Artificial Sequence [sequence omitted]

SEQ ID NO: 17 - protein, 10 aa, Artificial Sequence
  Description of Artificial Sequence Synthetic peptide
GGGGSGGGGS

SEQ ID NO: 18 - protein, 15 aa, Artificial Sequence
  Description of Artificial Sequence Synthetic peptide
GGGGSGGGGSGGGGS

SEQ ID NO: 19 - protein, 16 aa, Artificial Sequence
  Description of Artificial Sequence Synthetic peptide
GGGSGGGSGGGSGGGS

SEQ ID NO: 20 - protein, 4 aa, Artificial Sequence
  Description of Artificial Sequence Synthetic peptide
GGGS

SEQ ID NO: 21 - protein, 5 aa, Artificial Sequence
  Description of Artificial Sequence Synthetic peptide
SGGGG

SEQ ID NO: 22 - protein, 10 aa, Artificial Sequence
  Description of Artificial Sequence Synthetic peptide
SGGGGSGGGG

SEQ ID NO: 23 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 122 aa, Artificial Sequence [sequence omitted]

SEQ ID NO: 25 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 26 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 448 aa, Artificial Sequence
  Description of Artificial Sequence Synthetic polypeptide
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 11 aa, Artificial Sequence
  Description of Artificial Sequence Synthetic peptide
GASENIYHALN

SEQ ID NO: 29 - protein, 17 aa, Artificial Sequence
  Description of Artificial Sequence Synthetic peptide
EILPGSGHTEYTENFKD

SEQ ID NO: 30 - protein, 8 aa, Artificial Sequence
  Description of Artificial Sequence Synthetic peptide
DYKDDDDK

SEQ ID NO: 31 - protein, 6 aa, Artificial Sequence
  Description of Artificial Sequence Synthetic 6xHis tag
HHHHHH

SEQ ID NO: 32 - protein, 9 aa, Artificial Sequence
  Description of Artificial Sequence Synthetic peptide
YPYDVPDYA

SEQ ID NO: 33 - protein, 10 aa, Artificial Sequence
  Description of Artificial Sequence Synthetic peptide
GHIFSNYWIQ